(19) United States
(12) Patent Application Publication
(10) Pub. No.: US 2022/0119446 A1
(43) Pub. Date: Apr. 21, 2022

(54) BIOMARKERS AND TREATMENT OF NEURONAL INJURY AND NEURODEGENERATION

(71) Applicant: Board of Supervisons of Louisiana State University and Agricultural and Mechanical College, Baton Rouge, LA (US)

(72) Inventors: Nicolas G. BAZAN, New Orleans, LA (US); Jorgelina M. CALANDRIA, New Orleans, LA (US); Ludmila S. BELAYEV, Metairie, LA (US)

(21) Appl. No.: 17/294,769

(22) PCT Filed: Nov. 15, 2019
(86) PCT No.: PCT/US2019/061697

§ 371 Date: May 18, 2021

Related U.S. Application Data
(60) Provisional application No. 62/768,748, filed on Nov. 16, 2018.

(51) Int. Cl. C07K 7/06; G01N 33/68

(52) U.S. Cl. CPC C07K 7/06; A61K 38/00; G01N 33/6863

(57) ABSTRACT

The invention is directed to methods for identifying and treating neuronal injury or neurodegeneration.

This application is a National Stage Entry of International Patent Application No. PCT/US2019/061697, filed in the United States receiving office on 15 Nov. 2019, which claims priority from U.S. Provisional Application No. 62/768,748, filed on Nov. 16, 2018, the entire contents of each of which are incorporated herein by reference.

### GOVERNMENT INTERESTS

This invention was made with government support under Grant No. EY005121 awarded by the National Institutes of Health. The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### SEQUENCE LISTING

The instant application contains a Sequence Listing which has been submitted electronically in ASCII format and is hereby incorporated by reference in its entirety. Said ASCII copy, created on Dec. 27, 2019, is named 2932719-051-WO1_SL.txt and is 16,828 bytes in size.

### FIELD OF THE INVENTION

The invention is directed to methods for identifying and treating neuronal injury or neurodegeneration.

### BACKGROUND OF THE INVENTION

Neuronal injury and neurodegenerative diseases a significant socioeconomic burden for which adequate treatment is currently lacking.

Stroke is the fifth leading cause of death in the United States, killing nearly 130,000 people per year, costing the United States an estimated $34 billion each year. A severe head injury averages lifetime costs of over $3 million and for a moderate head injury over $1 million. For the population as a whole, it has been estimated that the costs for care of people with a new TBI is $6.5 billion per year and ongoing care for people with existing head injuries is $13.5 billion. Alzheimer's disease (AD) is by far the costliest of all these conditions. More than 5.3 million Americans suffer from the disease, and this year, for the first time, Alzheimer's-related health care costs will surpass a quarter of a trillion dollars. By 2030, these costs are projected to exceed $600 billion. Following AD, PD is the second most common neurodegenerative disorder in the United States, and the total economic impact of Parkinson's disease is $14.4 billion a year. A lack of treatment options for changing the trajectory of neurodegenerative diseases progression, in combination with an increasing elderly population, portends a rising economic burden on patients and taxpayers.

Thus, there is a need for appropriate biomarkers to further elucidate the pathophysiology of neuronal injury and neurodegenerative diseases and to aid in diagnosis, prognosis, and the evaluation of treatment efficacy. There also exists a significant need for therapies that effectively treat neuronal injury and neurodegenerative diseases.

### SUMMARY OF THE INVENTION

Aspects of the invention are directed towards a pharmaceutical composition or formulation comprising a therapeutically effective amount of an FZD5 receptor blocker (also referred to as an FZD5 receptor antagonist), a therapeutically effective amount of a long chain fatty acid, and a pharmaceutically acceptable carrier.

In embodiments, the long chain fatty acid comprises a docosanoid, an elovanoid, or a combination thereof. For example, the fatty acid derives from DHA, EPA, or omega-3.

Non-limiting examples of docosanoids comprise DHA, neuroprotectins (such as NPD1); lipoxin A4; DHA-derived Resolvins; Maresin 1; 10R, 17R diHDHA and its methyl ester derivatives; 10S, 17S diHDHA and its methyl ester derivatives; or any combination thereof.

Non-limiting examples of elovanoids comprise comprises mono-hydroxylated elovanoid, a di-hydroxylated elovanoid, an alkynyl mono-hydroxylated elovanoid, and an alkynyl di-hydroxylated elovanoid, or any combination thereof.

In embodiments, wherein the FZD5 receptor blocker comprises a peptide comprising SEQ ID NO: 1 (NH-Met-Asp-Gly-Cys-Glu-Leu-CO2H). For example, the peptide is N-terminally butyloxycarbonyl-(Boc) protected.

In embodiments, the peptide can comprise protective groups. For example, see U.S. Pat. Nos. 9,278,119, 8,497,352, U.S. Patent Application Publication US20080207521A1, and International Patent Application Publication WO2016092378A1, each of which are incorporate by reference herein in their entireties.

In embodiments, the FZD5 receptor blocker interacts with an extracellular domain of the FZD5 receptor. For example, the FZD5 receptor blocker interacts with N-terminus domain of the FZD5 receptor, such as the domain comprising amino acids 28 to 238. Without wishing to be bound by theory, a peptide such as that comprising SEQ ID NO: 1 can be responsible for the negative regulatory effect on the FZD5 receptor, co-receptors (such as ROR1, ROR2, RYK, LRP5, LRP6), putative dimerization partners (such as FZD1, FZD2, FZD 4, FZD7 and FZD8 (J Biol Chem. 2015 Mar. 13; 290(11):6789-98.)), soluble proteins (such as SFRP1, SFRP2, SFRP3, SFRP4 and SFRP5 (Kuhl, M. et al. (2000) Trends Genet. 16:279.; Wallingford, J. B. et al. (2000) Nature 405:81)), other factors that can interact with the Wnt-FZD complex (such as WIF1, PG, DKK1. CXXC4 and KREMEN1), or any combination thereof.

In embodiments, the composition further comprises a MicroRNA, for example miR-129-5p (Cell Death & Disease volume 9, Article number: 394 (2018)) or WNT5A antisense RNA 1 (WNT5A-AS1), which can prevent Wnt5a from being expressed.

Aspects of the invention are further directed towards a method for treating a patient afflicted with a condition characterized by neuronal damage and/or neuronal injury by administering to the patient the pharmaceutical formulation described herein.

Non-limiting examples of such conditions comprise retinal damage, ischemic stroke, Alzheimer's disease, traumatic brain injury (TBI), Parkinson's Disease, or age-related macular degeneration (AMD); Diabetic: retinopathy, glomerulonephritis and neuropathy; Psoriatic-arthritis, Crohn's disease, Rheumatoid-arthritis, Atherosclerosis, asthma, periodontitis, Hashimoto thyroiditis, Systemic lupus erythematosus, Multiple sclerosis, fibromyalgia and metabolic syndrome.

In embodiments, the FZD5 receptor blocker and the fatty acid are administered simultaneously. Without wishing to be bound by theory, fatty acids can enhance activity of the FZD5 receptor blocker by preventing expression of Wnt5a, and facilitating binding of the blocker to the receptor using the structural hydrophobic pocket suitable for fatty acids (see, for example, Proc Natl Acad Sci USA. 2017 Apr. 18; 114(16): 4147-4152).

In embodiments, the FZD5 receptor blocker and the fatty acid are administered as a single-dose pharmaceutical composition/formulation.

In embodiments, the FZD5 receptor blocker and/or the fatty acid are administered enterally (for example orally or rectally) or parenterally (for example, intravascular administration; intravitreal and subretinal injection or delivery; subcutaneous injection; subcutaneous deposition; intramuscular injection; intraperitoneal injection; transdermal, nasal and inhalational).

In embodiments, the neuronal damage or neuronal injury is the result of or exacerbated by uncompensated oxidative stress.

In embodiments, the method further comprises the steps of: obtaining a sample from the patient; measuring the protein level of Wnt5a protein in the sample and comparing the protein level of Wnt5a protein in the sample to a control sample; herein the patient is treated if the protein level of Wnt5a protein is changed compared to the control.

In embodiments, the method further comprises the step of diagnosing the patient as having a condition characterized by neuronal injury or neuronal damage if the protein level of Wnt5a protein in the sample is higher than that of the control sample.

Aspects of the invention are still further directed towards a method for determining the presence of neuronal injury or neuronal damage in a patient. In embodiments, the method comprises obtaining a sample from a patient; measuring the protein level of Wnt5a protein in the sample and comparing the protein level of Wnt5a protein in the sample to a control sample; wherein the patient is afflicted with neuronal injury or neuronal damage if the level of Wnt5a protein in the sample is changed as compared to the control.

In embodiments, a higher protein level of Wnt5a protein in the sample relative to the control sample is indicative of a neurodegenerative disease.

For example, embodiments can be drawn to a method of treating a neuronal injury or neuronal damage in a subject at risk thereof comprising: obtaining from a biological sample taken from the subject at risk of neuronal injury or neuronal damage an expression level of Wnt5a; determining that the subject has or is at risk of having a neuronal injury or neuronal damage based on the expression level or protein level of Wnt5a compared to a control expression level or protein level, wherein said control expression level or protein level is from a patient that does not have a neuronal injury or neuronal damage, and administering to the subject a therapeutically effective amount of a treatment regiment. In embodiments, the treatment regiment can be those described herein.

Still further, aspects of the invention are directed towards s method for determining the prognosis of a patient suffering from a condition characterized by neuronal injury or neuronal damage. In embodiments, the method comprises the steps of obtaining a sample from a patient; measuring the expression level of Wnt5a protein in the sample and comparing the expression level of Wnt5a protein in the sample to a control sample; and determining the prognosis of the patient.

In embodiments, the measuring step is repeated at one or more intervals.

In embodiments, the measuring comprises Western blot, ELISA (enzyme linked immunosorbent assay), radioimmunoassay analysis (RIA), radial immunodiffusion, Ouchterlony immunodiffusion, rocket immunoelectrophoresis, tissue immunohistochemistry, immunoprecipitation assays, complement fixation assays, flow cytometry, and protein chip (protein microarray), capillary western blot, protein MS, Protein sequencing, HPLC, gas chromatography.

In embodiments, the sample comprises tissue biopsy, stool, plasma, cord blood, neonatal blood, cerebral spinal fluid, tears, vomit, saliva, urine, feces, or meconium. As an example, the sample can comprise blood sample that is separated into plasma before measuring.

In embodiments, the control sample comprises a sample from a normal subject and/or a sample isolated from the patient prior to the onset of the disease.

In embodiments, the subject has suffered a stroke. For example, the protein level of Wnt5a protein is measured within seven days, five days, or three days of the stoke event.

In embodiments, the neurodegenerative diseases comprises uncompensated oxidative stress.

Further, aspects of the invention are directed towards methods of treating a subject with neuronal injury or neuronal damage comprising administering to a subject in need of such treatment an effective amount of a therapeutic agent, for example a therapeutic agent described herein, wherein the subject has been determined to have an elevated level of Wnt5a expression and/or protein level in a sample obtained from the subject before the treatment.

Still further, aspects of the invention are directed towards a diagnostic kit for determining brain injury status in a patient. In embodiments, the diagnostic kit comprises a substrate for collecting a sample from the patient; and means for measuring the protein level of Wnt5a protein. For example, the means for measuring comprises Western blot, ELISA (enzyme linked immunosorbent assay), radioimmunoassay analysis (RIA), radial immunodiffusion, Ouchterlony immunodiffusion, rocket immunoelectrophoresis, tissue immunohistochemistry, immunoprecipitation assays, complement fixation assays, flow cytometry, and protein chip (protein microarray). capillary western blot, protein MS, Protein sequencing, HPLC, gas chromatography.

### BRIEF DESCRIPTION OF THE FIGURES

FIG. 1 shows docosanoids counteract UOS-dependent NF-κB activation and apoptosis via Wnt5a/FZD5/ROR2. (A) shows the experimental design and morphology criteria to identify apoptotic cells. (B) shows apoptotic cell percentage measured using Hoechst staining. (C) shows the chemical structure of Docosahexaenoic acid (DHA) and a number of its docosanoids. (D) shows the quantification of Wnt5a expression determined using real time PCR in human primary RPE cells following uncompensated oxidative stress (UOS). (E) shows the quantification of FZD5 expression determined using real time PCR in ARPE-19 cells following UOS. (F) shows the quantification of Wnt5a expression and FZD5 expression determined using real time PCR in human primary RPE cells following UOS. (G) shows a schematic representation of a luciferase assay performed with TOPFlash/FOPFlash, and NF-κB/p65 reporter constructs. FIG. 1G discloses SEQ ID NOS 79-80, respectively, in order of appearance. (H-J) show luciferase reporter system assay data.

FIG. 2 shows NPD1-dependent cRel binding to promoter A decreases Wnt5a expression. (A) shows a schematic representation of siRNA resistant cREL ORF strategy to control off target effects of siRNA. FIG. 2A discloses SEQ ID NO: 81. (B) shows cRel quantification (top) and Wnt5a (lower) mRNA by means of SYBR green-based real-time PCR in hRPE cells undergoing UOS, +/−NPD1. (C) shows Wnt5a mRNA quantification of non-transfected cells as a control for FIG. 2B. (D & F) provide a model of regulation of NF-κB sites by cRel obtained using TRED software. (E) shows quantification of SYBR green-based real-time PCR to detect differential binding of cRel to the genomic DNA in the presence of Wnt5a and NPD1.

FIG. 3 shows secreted Wnt5a is reduced by NPD1 in human RPE cells undergoing UOS. (A-D) show western blot analysis of Wnt5a protein release from ARPE-19 hRPE cells in the presence or absence of NDP1. (E) provides a schematic representation of an exosome enrichment protocol using ultracentrifugation. (F) shows a western blot analysis of the pellets obtained from the various fractions identified in FIG. 3E.

FIG. 4 shows NPD1 enhances Wnt5a internalization. A-F shows fluorescence microscopy images showing co-localization of FZD5 and Wnt5a in hRPE cells and associated quantification of the images.

FIG. 5 shows Pitstop2 halts the internalization of Wnt5a and its subsequent activation of NF-κB. (A) provides an experimental design to determine the mechanisms of Wnt5a/FZD5 colocalization and internalization. (B) provides quantification of clusters showing colocalization in the absence of Pitstop2. (C) provides quantification of clusters showing colocalization in the presence of Pitstop2. (D) shows Western blot analysis of Wnt5a expression in response to UOS in the presence or absence of NPD1 and Pitstop2. (E) provides reporter assay data showing the effects of Pitstop2 on NF-κB activation. (F-G) show fluorescence images. (H) shows a model for the internalization and recycle of Wnt5a and FZD5 to activate NF-κB/p65.

FIG. 6 shows DHA prevents Wnt5a overexpression and secretion in response of ischemia reperfusion. (A) provides an experimental design for transient middle cerebral artery occlusion (MCAo) and subsequent neurological testing and experimental analysis. (B) shows the effect of DHA administration on neurological recovery following MCAo. (C) shows the effect of Box5 administration on total neurological score following MCAo. (D) provides MRI quantification data showing the effect of Box5 administration on infarct size following MCAo. (E) shows representative coronal of T2 weighted image (T2WI) in the first column, the defined core and penumbra region (red and blue, respectively) in the second column; and a 3D reconstruction of the lesion in the third column. (F) shows Wnt5a mRNA assessment by means of SYBR-green real-time PCR in rat cortex ipsilateral (Ipsi) or contralateral (Contra) of MCAo, treated with saline (vehicle) or DHA. (G) shows Wnt5a protein in plasma in the presence or absence of post-surgical administration of DHA at 1, 3, and 7 days following MCAo. (H) shows Wnt5a protein in plasma in the presence or absence of post-surgical administration of Box5 at 1, 3, and 7 days following MCAo. (I) provides western blot analysis Ipsi and Contra tissue in Saline and DHA treated animals 1 day after MCAo or Sham-MCAo. (J) shows the quantification of Wnt5a and NF-κB linked gene expression in MCAo. (K) provides a schematic of Wnt5a inflammatory signaling after increased abundance due to ischemia/reperfusion.

FIG. 7 shows the effects of UOS and NPD1 treatment on Wnt5a mRNA expression in 15-LOX-1-deficient ARPE-19 cells from microarray data. Wnt5a was up-regulated in ARPE-19 cells deficient in 15-LOX-1 undergoing UOS and downregulated by NPD1.

FIG. 8 shows the quantification of mRNA of Wnt5a in primary human RPE cells using real-time PCR.

FIG. 9 provides the quantification of apoptosis in the presence or absence of Wnt5a following UOS of ARPE-19 cells.

FIG. 10 provides (A-C) a design of luciferase reporter assay performed with COX-2 promoter construct and the associated quantification of the luciferase activity.

FIG. 11 shows (A-B) the quantification of mRNA of FZD5 and ROR2 in silenced cells.

FIG. 12 shows (A-D) experimental data validating the FZD5 antibody for use in immunocytochemistry.

FIG. 13 shows a Pearson's colocalization coefficient (PCC) for the first experiment in the series of colocalization by immunocytochemistry, related to FIGS. 4 and 5.

FIG. 14 shows a Pearson's colocalization coefficient (PCC) for the experiment in FIG. 4C.

FIG. 15 shows a Pearson's colocalization coefficient (PCC) for the experiment in FIG. 5 (B and C).

FIG. 16 shows a schematic representation of the mechanisms by which DHA and BOX5 alleviate Wnt5a-mediated cell damage.

FIG. 17 shows western blot expression of vesicular (3000×g SN) and secreted (100,000×g SN) Wnt5a in the postmortem brains of two Alzheimer Disease (AD) patients as compared to a human control.

FIG. 18 shows docosanoids counteract UOS-dependent NFkB activation and apoptosis. (A) Design to determine apoptotic cells. (B) DHA (i), and its derivatives: NPD1 (ii), 10R, 17R diHDHA (iii), Maresin-1 (iv), RvD1 (v) and RvD2 (vi) counteracted these effects. (D) Wnt5a enhanced cell death triggered by H2O2. Apoptosis measured using Hoechst staining and ImageJ. (C, E, F) Docosanoids prevented Wnt5a transcription increases in cells undergoing UOS. SYBR green real time PCR was used to determine semi quantitatively Wnt5a (C) and FZD5 (E) in hRPE cells and receptors linked to Wnt signaling (F) in ARPE-19 c induce a decrease of UOS-triggered Wnt5a transcription. Standardization was performed using β-actin and GAPDH. (G) TOPFlash/FOPFlash and NFkB/p65 reporter constructs. FIG. 18G discloses SEQ ID NOS 79-80, respectively, in order of appearance. (H-J) Luciferase assay using TOP-Flash (wild type) and FOP-flash (mutated) to detect β-catenin activation (H) and NFkB activation (I-J) when Wnt5a was added to the medium of hpRPE cells. Standardization was made using a plasmid expressing GFP. (J) hRPE cells were transfected with siRNA targeting FZD5 and ROR2 separately and together or control non-specific siRNA. UOS was induced+/−100 nM NPD1 and Wnt5a. Bars are mean of 3 measurements and the standard error of the mean. *p<0.05.

FIG. 19 shows NPD1-dependent cRel binding to promoter A decreases Wnt5a expression. (A) Without wishing to be bound by theory, schematic indicating a role of cREL in the modulation of the expression of Wnt5a. FIG. 19A discloses SEQ ID NO: 81. (B-C) Quantification of cRel (B) and Wnt5a (C) mRNA by the means of SYBR green-based real-time PCR in hpRPE cells undergoing UOS, +/−NPD1. (B) Cells transfected with cREL ORF were exposed to UOS in the presence or absence of NPD1 for 4 hours. cREL (left) and Wnt5a (right) mRNA was quantified by real-time PCR. (C) Wnt5a mRNA quantification of non-transfected cells (Control for B). (D and F) Without wishing to be bound by theory, regulation of NFkB sites by cRel: in silica analysis of Wnt5a promoter (Katula et al., 2012) showing that the 2 binding sites for NFkB have high affinity for p65, p50 and cRel. The cartoon shows directions in which transcription factors act. NFkB site prediction is in Table S1. Other NFkB binding sites detected by TRED. Region 2 corresponds to upstream NFkB binding site and Region 6 to the downstream binding site depicted in D and F. Regions 1, 3, 4, 5, and 7 showed up in the general TRED search with high score (Table S1) for the 3 NFkB. Four amplicons were designed close or sitting on these regions to assess each site. In purple CpG islands that encompass the putative binding sites are depicted (Table S2). (E) SYBR green-based real-time PCR using as template the proteinase digested genomic DNA fragments resulting from micrococcal DNAs digestion and cRel pull down. UOS=1600 μM H₂O₂ plus 10 ng/ml TNFα. NPD1=100 nM and Wnt5a 50 ng/ml unless stated otherwise. The bars represent mean of 3 measurements and standard error of the mean. *p<0.05.

FIG. 20 shows secreted Wnt5a and FZD5 is reduced by NPD1 in human RPE cells undergoing UOS. Wnt5a protein is released from ARPE-19 (A-B) and hRPE cells (D) under UOS. (A) ARPE-19 cells were treated with 600 μM H₂O₂ and 10 ng/ml TNFα for 6 hours in the presence or absence of 100 nM NPD1. Wnt5a was measured in cellular lysate (cWnt5a) and in medium (sWnt5a) by the means of western blot. (B) Cellular content of Wnt5a in 15-LOX-1d and control cells. (C) FZD5 was measured in hpRPE cells undergoing UOS in treated with 100 nM NPD1 or 100 ng/ml Box5 in the presence or absence of 50 ng/ml Wnt5a. The bands were standardized by total protein stain (see herein for description) (D) Deglycosylation of Wnt5a secreted by hpRPE cells. The secreted Wnt5a protein was concentrated from the medium by Chloroform/Methanol precipitation. The pellet was resuspended and digested with N and O-glycosylases (Degly). In parallel, non-digested samples (Gly) were ran. Western blots were replicated using 2 different antibodies and a positive control (FIG. 26). (E-F) Content of sWnt5a in medium of human RPE cells in the presence of 1600 μM H₂O₂ and 10 ng/ml TNFα. (E) Exosome enrichment protocol using ultracentrifugation. (F) Content of sWnt5a in the different fractions of the medium. The bars represent the mean of 3 measurements and the standard error of the mean. *p<0.05.

FIG. 21 shows Wnt5a and FZD5 are internalized via CME. (A) Blow up of a single cell showing vesicles positives to Wnt5a (red), FZD5 (green) or both yellow. The fourth panel shows a nucleus drawing (blue) and the position of vesicles showing colocalization of FZD5 and Wnt5a. (B) Quantification of colocalized spots in RPE cells undergoing UOS+/−lipids in FIG. 18C. Pearson colocalization coefficient was plotted in Figures S4D. Bars: mean of 3 measurements and standard error of the mean. * p<0.05. (C) Blow up of a cell showing large cluster of Wnt5a signal (Red) present most frequently in certain treatments. (D, E) Box plot representation of the size (D) and the mean of the intensity per vesicle (E) obtained by analyzing the images of hpRPE cells undergoing UOS and treated with 100 nM NPD1 or 100 ng/ml Box5 in the presence or absence of external Wnt5a or overexpressing the Wnt5a variant 1. Using IMARIS software we analyzed the vesicles that showed colocalization of Wnt5a/FZD5, FZD5/Clathrin and Wnt5a/Clathrin. One-way ANOVA and multiple comparisons honest difference test (Tukey's range test) was applied. The boxes depict the median (middle bar) Quartile 1 and 3 (upper and lower limit of the box) and the maximum and minimum range of observations (bars). (F) Upper panels show the IMARIS rendering of the vesicle in: yellow=Wnt5a/FZD5 colocalization; red=FZD5 only and Green=Wnt5a only signals to show the variation in number and sizes. Lower panel actual image analyzed in upper panel. (G) Intensity of the vesicles depicted in (F) were plotted along with the nuclei (DAPI=blue), and the box plot over-imposed shows the median Q1,Q3 and the minimum and maximum observations.

FIG. 22 shows Pitstop2 halts activation of NFkB. (A) Experimental design. (B) Pitstop2 interferes with the activation of NFkB/p65. Reporter assay of 3 NFkB/p65 binding sites in tandem driving the expression of luciferase ORF, the construct was depicted in FIG. 1G. NPD1=100 nM and UOS=1600 μM H2O2. (C) High magnification image of cell subjected to 25 μM Pitstop2. Red=Wnt5a. (D) Western blot analysis of ROR2, FZD5 and Wnt5a in hpRPE cells overexpressing ROR2-His tag ORF, FZD5 ORF, ROR2-His tag and FZD5 ORFs together and Wnt5a. (E) IMARIS analysis of the vesicles observed by immunocytochemistry targeting green=Wnt5a, red=FZD5. The mean of the intensity for the vesicles observed in hpRPE cells under UOS in the presence or absence of NPD1, when the cells were expressing the ORFs (open reading frame) tested by WB in (D). Colocalization vesicles are depicted in yellow. The box plot over-imposed shows the median, Q1,Q3 and the minimum and maximum observations. (G) Vesicle like signal in the Z axe of the Z-stack. Whole arrow shows a fusion between a FZD5 and Wnt5a positive to a large Wnt5a-positive cluster. Arrowhead shows already fused colocalized cluster. (H) Model of internalization and recycle of Wnt5a and FZD5 to activate NFkB/p65. The bars represent the mean of 3 measurements and the standard error of the mean. *p<0.05.

FIG. 23 shows neuroprotection by DHA that prevents Wnt5a overexpression and secretion in response of brain ischemia reperfusion and by Box5 that blocks its action. (A) DHA, Box5 or saline were administered at 1 h after 2 h of MCAo and rats sacrificed on days 1, 2, 3 or 7. (B) Neurological recovery. Total score (normal score=0, maximal deficit=12), tactile placing (dorsal, lateral, proprioceptive reactions; normal score=0, maximal deficit=2). DHA or saline was administered at 1 h after 2 h of MCAo and rats sampled on days 1, 2, 3 or 7. Values are mean±SD; n=4 rats/group. *Significantly different from saline group (p<0.05, repeated measures ANOVA followed by Bonferroni tests). (C-E) 400 μg Box5 IV administration, 1 h after MCAo (C) Total neurological score at days 1, 3 and 7; (D) MRI quantification at day 7 of lesion volume depicting total, core and penumbra and; (E) representative coronal sections showing T2 weighted image (T2WI), the defined core and penumbra region (red and blue, respectively) in the second column; and a 3D reconstruction of the lesion. (F) Wnt5a mRNA assessment by SYBR-green real-time PCR in rat cortex Ipsilateral (Ipsi) or contralateral (Contra) of MCAo, treated with saline (vehicle) or DHA. (G and H) Wnt5a protein in plasma 2 h after MCAo with DHA (N=4); (G) or Box5 (N=4); (H) at 1, 3 and 7 days post-surgery. (I) Western blot of A1 and A2 (Ipsi and Contra) in Saline and DHA treated animals 1 day after MCAo or Sham-MCAo. (J) Wnt5a and NFkB linked gene expression in MCAo. MCAo and saline (vehicle) and DHA for 3 days (N=3), each reaction was run in triplicate. mRNA measured using SYBR green RT-PCR. Color-coded region to test gene expression is depicted. Bars represent t mean of 3 measurements and standard error of the mean. *p<0.05. (K) Wnt5a inflammatory signaling after increased abundance due to ischemia/reperfusion.

FIG. 24 shows Wnt5a was upregulated in ARPE-19 cells deficient in 15-LOX-1 undergoing UOS and downregulated by NPD1. (A) Design for experiments leading to the identification of Wnt5a. UOS-triggered increase in Wnt5a expression was reversed by NPD1 in 15-LOX-1d. 15-LOX-1d cells, which shows depletion in NPD1 synthesis (Calandria et al., 2009), were used to determine genes regulated by the lipid messenger in a microarray assay. UOS=600 μM H2O2 plus 10 ng/ml TNFα Representative values of 3 independent experiments. ANOVA and test for false positives was applied to select regulated genes on microarray output. (B) Quantification of mRNA of Wnt5a in primary human RPE cells. Confirmation of the NPD1-regulation of Wnt5a transcription. UOS was carry out using 1600 μM H2O2 for hRPE cells, plus 10 ng/ml TNFα to confirm microarray output using SYBR green-based real-time PCR in human primary cells. Representative values of 3 independent experiments. Bars represent the mean+standard error of the mean of 3 different experimental subjects. (C) Wnt5a enhances the percentage of cell death induced by UOS in ARPE-19 cells. Hoechst-positive ARPE-19 cells were beyond H2O2-induced levels. The criteria used to determine Hoechst-positive cells and the experimental design is depicted in FIG. 18A. The bars represent the mean of 3 measurements and the standard error of the mean. *p<0.05.

FIG. 25 shows quantification of mRNA of FZD5 and ROR2 in silenced cells. (A) FZD5 and (B) ROR2 mRNA quantification on Negative control, FZD5 and FZD5 plus ROR2 siRNA transfected human RPE cells. Controls corresponding to experiment. The bars represent the mean of 3 measurements and the standard error of the mean. *p<0.05.

FIG. 26 shows deglycosylation of Wnt5a secreted by hpRPE cells. (A and B) Medium from hpRPE cells undergoing UOS were incubated with recombinant Wnt5a (lanes 2-9) in the presence or absence of Box5 or NPD1. Lane 10 and 11, medium from cells overexpressing Wnt5a ORF. Lane 12=recombinant Wnt5a (R&D). Lane 1=Rainbow fluorescent marker (GE). The secreted Wnt5a protein was concentrated from the medium by Chloroform/Methanol precipitation. The pellet was resuspended and digested with N and O-glycosylases (Degly). In parallel, non-digested samples (Gly) were ran. Western blots were replicated using 2 different antibodies and a positive control. The antibody used against Wnt5a was the Monoclonal clone #3D10 from Life Technologies/Thermo Fisher Scientific Cat #MA5-15511 (A) and Monoclonal Rat IgG2A Clone #442625 from R&D cat #MAB645 (B). Positive control=Recombinant Human/Mouse Wnt-5a Protein produced in Chinese Hamster Ovary cell line, CHO-derived Wnt-5a protein (Gln38-Lys380).

FIG. 27 shows colocalization detection of FZD5 and Wnt5a signal using ImageJ. (A) Representative images of colocalized signal of FZD5 and Wnt5a in hRPE cells. Cells were incubated for 2 hours with 1600 μM H₂O₂+/−100 nM NPD1 and 50 ng/ml Wnt5a. Immunostaining of Wnt5a (Red) and FZD5 (Green) and z-stack images using BioImageXD (20×). Lasers set up using Control+Wnt5a and used without modification to take remaining pictures. Colocalization of the 2 signals are in left column (white). (B) Total signal intensity of 3 channels (DAPI=blue, Alexa 488=green and Alexa 594=red) in 3 random fields/well/condition. On the right of each row, histogram of intensity vs frequency depicts pixels numbers showing intensity value on X-axis. The upper limit intensity is set at 4095. Black vertical lines for each channel indicate the mode (most frequent observation) to designate the intensity at which each curve reaches its maximum. Signal points or clusters of pixels showing colocalization (A-left column) were quantified using ImageJ of 3-6 random fields encompassing 1 or 2 wells, in up to 3 independent experiments. (C) Frequency vs Area histogram for representative fields showing different sizes of clusters of Wnt5a positive signal. (D) Pearson's colocalization coefficient (PCC) for first experiment in the series of colocalization by immunocytochemistry. The PCC values obtained for all slices of z-stacks of 3 fields were averaged and plotted. Bars represent the mean of 3 measurements and the standard error of the mean. *p<0.05. (E-H) Validation of FZD5 antibody for Immunocytochemistry. Histograms of Intensity vs frequency for FZD5 signal (red) and siRNA tracer signal (black) (E and F). (G and H) Representative pictures of human RPE cells transfected with G, FZD5 siRNA and H, Negative control siRNA. To allow the comparison confocal lasers were set to the negative control parameters and pictures were taken without changing them. White=tracer siRNA; blue=DAPI and red=FZD5.

### DETAILED DESCRIPTION OF THE INVENTION

### Abbreviations and Definitions

Detailed descriptions of one or more embodiments are provided herein. It is to be understood, however, that the invention may be embodied in various forms. Therefore, specific details disclosed herein are not to be interpreted as limiting, but rather as a basis for the claims and as a representative basis for teaching one skilled in the art to employ the invention in any appropriate manner.

The singular forms “a,” “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be non-limiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises,” “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Various exemplary embodiments of the invention comprise a biomarker of retina damage, brain damage, neurodegeneration, other neuronal diseases or disorders, or a combination thereof. In certain embodiments, Wnt5a serves as a biomarker for neural injury, neurodegeneration, other forms of neuronal damage or stress, or a combination thereof. Wnt5a can be increased in AD patients, following traumatic brain injury, or ischemic stroke.

In embodiments, BOX5, DHA, docosanoids, elovanoids or a combination thereof can reduce Wnt5a-mediated cellular damage. DHA, docosanoids, or other structurally similar signaling molecules can be used to reduce Wnt5a-mediated cellular damage. In embodiments, Wnt5a-mediated cellular damage is mediated through administration of an elovanoid. Elovanoids can comprise fatty acids that further comprise 32 or 34 carbons.

Pharmaceutical Combinations/Formulations and Routes of Administration

Aspects of the invention are directed towards compositions and formulations comprising a therapeutically effective amount of an FZD5 receptor blocker, a therapeutically effective amount of a long chain fatty acid, and a pharmaceutically acceptable carrier.

“Long chain fatty acids” refer to a fatty acid with carbon chains comprising 14 or more carbons. “Very long chain fatty acids” refer to a fatty acid with carbon chain comprising 23 to 42 carbons. Recent investigations have shown that certain polyunsaturated fatty acids (PUFA) are enzymatically converted to bioactive derivatives that play important roles in inflammation and related conditions. Notable among these are the omega-3 (n3) fatty acids containing 22 carbons including eicosapentaenoic acid (EPA or C20:5n3) (20 carbons, 5 double bonds, omega-3), docosapentaenoic acid (DPA or C22:5n3), and especially docosahexaenoic acid (DHA or C22:6n3) (22 carbons, 6 double bonds, omega-3). These PUFA are converted via lipoxygenase-type enzymes to biologically active hydroxylated PUFA derivatives. Most important among these are specific types of hydroxylated derivatives that are generated in certain inflammation-related cells via the action of a lipoxygenase (LO) enzyme (e.g. 15-LO, 12-LO), and result in the formation of mono-, di- or tri-hydroxylated PUFA derivatives with potent actions including anti-inflammatory, pro-resolving, neuroprotective or tissue-protective actions, among others. For example, neuroprotectin D1 (NPD1), a dihydroxy derivative from DHA formed in cells via the enzymatic action of 15-lipoxygenase (15-LO) was shown to have a defined R/S and Z/E stereochemical structure (10R,17S-dihydroxy-docosa-4Z,7Z,11E,13E,15Z,19Z-hexaenoic acid) and a unique biological profile that includes stereoselective potent anti-inflammatory, homeostasis-restoring, pro-resolving, bioactivity. NPD1 has been shown to modulate neuroinflammatory signaling and proteostasis, and to promote nerve regeneration, neuroprotection, and cell survival.

Other important types of omega-3 fatty acids are the omega-3 very-long-chain polyunsaturated fatty acids (n3 VLC-PUFA or VLC-PUFA), which are produced in cells containing elongase enzymes that elongate PUFA with lower number of carbons to VLC-PUFA containing between 24 to 36 carbons. Representative types of VLC-PUFA include C32:6n3 (32 carbons, 6 double bonds, omega-3), C34:6n3, C32:5n3, and C34:5n3, which are biogenically derived through the action of elongase enzymes, such as ELOVL4 (ELOngation of Very Long chain fatty acids 4). These fatty acids are also acylated in complex lipids including sphingolipids and phospholipids such as those in certain molecular species of phosphatidyl choline. Without wishing to be bound by theory, these VLC-PUFA display functions in membrane organization, and their significance to health is increasingly recognized. The biosynthesis and biological functions of VLC-PUFA have been the subject of a number of recent investigations that have indicated potential roles in certain diseases.

In embodiments, the fatty acid comprises a docosanoid, which are signaling molecules made by the metabolism of twenty-two-carbon fatty acids, such as the omega-3 fatty acid DHA. Non-limiting examples of docosanoids comprise neuroprotectins, such as neuroprotection D1 (NPD1) and NPD1 derivatives (such as methyl-ester derivatives of NPD1); lipoxin A4; DHA-derived Resolvins (such as RvD1 (7S,8R,17S-trihydroxy-DHA), RvD2 (7S,16R,17S-trihydroxy-DHA), RvD3 (4S,7R,17S-trihydroxy-DHA), RvD4 (4S,5,17S-trihydroxy-DHA), RvD5 (7S,17S-dihydroxy-DHA), RvD6 (4S,17S-dihydroxy-DHA); Maresin 1 (MaR1); 10R, 17R diHDHA and its methyl ester derivatives; 10S,17S diHDHA and its methyl ester derivatives; or any combination thereof.

In embodiments, the fatty acid comprises an elovanoid, which are a class of lipid mediators that are oxygenated derivatives of VLC-PUFAs. ELVs have structures reminiscent of docosanoids but with different physicochemical properties and alternatively-regulated biosynthetic pathways. See, for example, Jun, Bokkyoo, et al. “Elovanoids are novel cell-specific lipid mediators necessary for neuroprotective signaling for photoreceptor cell integrity.” Scientific reports 7.1 (2017): 5279, which is incorporated by reference in its entirety. In embodiments, the elovanoid comprises mono-hydroxylated elovanoid, a di-hydroxylated elovanoid, an alkynyl mono-hydroxylated elovanoid, and an alkynyl di-hydroxylated elovanoid, or any combination thereof.

Members of the ‘frizzled’ gene family encode 7-transmembrane domain proteins that are receptors for Wnt signaling proteins. The FZD5 receptor protein is the receptor for the Wnt5A ligand. The term “receptor blocker” or “antagonist” can refer to a class of compounds or molecules which bind to a receptor with some affinity, but are unable to activate the receptor to provide an effect (and thus blocks the receptor's activation). The antagonist can be compared to a key which is able to slide into a lock, but is unable to turn in the lock to open it. In embodiments, the FZD5 receptor blocker comprises a peptide comprising SEQ ID NO: 1 (NH-Met-Asp-Gly-Cys-Glu-Leu-CO2H). In embodiments, the peptide is N-terminally butyloxycarbonyl-(Boc) protected. See, for example, BENEDETTI, ETTORE, et al. “Preferred conformation of the tert-butoxycarbonylamino group in peptides.” International journal of peptide and protein research 16.2 (1980): 156-172, which is incorporated by reference herein.

The FZD5 receptor blocker can interact with the extracellular domain of the receptor. For example, the FZD5 receptor blocker interacts with the receptor in the region between amino acids 28 to 238.

In embodiments, the composition can further comprise a synthetic or recombinant nucleotide, such as a microRNA. microRNAs (miRNAs) are short non-coding RNAs that are involved in post-transcriptional regulation of gene expression in multicellular organisms by affecting both the stability and translation of mRNAs. For example, formulations herein can further comprise MicroRNA-224 (see NCBI Reference Sequence: NR_029638.1).

For clinical applications, it can be necessary to prepare pharmaceutical compositions in a form appropriate for the intended application. For example, this can entail preparing compositions that are essentially free of pyrogens, as well as other impurities that can be harmful to humans or animals. Non-limiting embodiments are described herein.

The phrase “pharmaceutically or pharmacologically acceptable” can refer to molecular entities and compositions that do not produce adverse, allergic, or other untoward reactions when administered to an animal or a human. As used herein, “pharmaceutically acceptable carrier” can include any and all solvents, dispersion media, coatings, antibacterial and antifungal agents, isotonic and absorption delaying agents and the like. The use of such media and agents for pharmaceutically active substances is well known in the art. Supplementary active ingredients also can be incorporated into the compositions.

Administration of these compositions according to the invention will be via any route so long as the target tissue is available via that route. This includes, for example, intradermal, subcutaneous, intramuscular, intraperitoneal or intravenous injection. Compositions can normally be administered as pharmaceutically acceptable compositions, described supra.

Pharmaceutical formulations can also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In embodiments, the form can be sterile and can be fluid to the extent that easy administration by a syringe is possible. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, isotonic agents can be included, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Dispersions are prepared by incorporating the various sterilized active ingredients into a sterile vehicle which contains the basic dispersion medium and the required other ingredients from those enumerated above. In the case of sterile powders for the preparation of sterile injectable solutions, the methods of preparation can be vacuum-drying and freeze-drying techniques which yield a powder of the active ingredient plus any additional ingredient from a previously sterile-filtered solution thereof.

For oral administration the polypeptides of the invention can be incorporated with excipients that can include water, binders, abrasives, flavoring agents, foaming agents, and humectants.

The pharmaceutical combinations/formulation of the invention comprise compounds as described herein, such as an FZD5 receptor blocker and a long chain fatty acid, in an admixture along with a pharmaceutically acceptable carrier prepared according to conventional pharmaceutical techniques. As used herein, “pharmaceutically acceptable carrier” can include any and all solvents, dispersion media, coatings, antibacterial and antifungal agents, isotonic and absorption delaying agents and the like. Non-limiting examples of pharmaceutically acceptable carriers comprise solid or liquid fillers, diluents, and encapsulating substances, including but not limited to lactose, dextrose, sucrose, sorbitol, mannitol, xylitol, erythritol, maltitol, starches, gum acacia, alginate, gelatin, calcium phosphate, calcium silicate, cellulose, methyl cellulose, microcrystalline cellulose, polyvinylpyrrolidone, water, methyl benzoate, propyl benzoate, talc, magnesium stearate, and mineral oil. The amount of the carrier employed in conjunction with the combination is sufficient to provide a practical quantity of material per unit dose of the formulation.

The use of such media and agents for pharmaceutical active substances is well known in the art. Except insofar as any conventional media or agent is incompatible with the active ingredient, its use in the therapeutic compositions is included herein. Supplementary active ingredients can also be incorporated into the compositions.

The compositions of the invention can be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Pharmaceutically acceptable carriers for oral administration comprise sugars, starches, cellulose and its derivatives, malt, gelatin, talc, calcium sulfate, vegetable oils, synthetic oils, polyols, alginic acid, phosphate buffer solutions, emulsifiers, isotonic saline, and pyrogen-free water. Pharmaceutically acceptable carriers for parenteral administration comprise isotonic saline, propylene glycol, ethyl oleate, pyrrolidone, aqueous ethanol, sesame oil, corn oil, and combinations thereof.

Various oral dosages forms can be employed, non-limiting examples of which comprise solid forms such as tablets, capsules, granules, suppositories and/or powders. Tablets can be compressed, tablet triturates, enteric-coated, sugar-coated, film-coated or multiple compressed, containing suitable binders, lubricants, diluents, disintegrating agents, coloring agents, flavoring agents, flow-inducing agents, and melting agents. Liquid oral dosage forms comprise aqueous solutions, emulsions, suspensions, syrups, aerosols and/or reconstituted solutions and/or suspensions. The composition can alternatively be formulated for external topical application, or in the form of a sterile injectable solution.

The dosage can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect to be achieved; and rate of excretion.

A therapeutically effective dose can depend upon a number of factors known to those of ordinary skill in the art. The dose(s) can vary, for example, depending upon the identity, size, and condition of the subject or sample being treated, further depending upon the route by which the composition is to be administered, if applicable, and the effect which the practitioner desires. These amounts can be readily determined by the skilled artisan.

Pharmaceutically effective formulations can be provided as a composition comprising between 0.1 and 2000 mg/kg of a compound as described herein, such as an FZD5 receptor blocker or a long chain fatty acid. For example, pharmaceutically effective formulations can be provided as a composition comprising about 0.1 mg/kg, 1 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 225 mg/kg, 250 mg/kg, 275 mg/kg, 300 mg/kg, 325 mg/kg, 350 mg/kg, 375 mg/kg, 400 mg/kg, 425 mg/kg, 450 mg/kg, 475 mg/kg, 500 mg/kg, 525 mg/kg, 550 mg/kg, 575 mg/kg, 600 mg/kg, 625 mg/kg, 650 mg/kg, 675 mg/kg, 700 mg/kg, 725 mg/kg, 750 mg/kg, 775 mg/kg, 800 mg/kg, 825 mg/kg, 850 mg/kg, 875 mg/kg, 900 mg/kg, 925 mg/kg, 950 mg/kg, 975 mg/kg, 1000 mg/kg, 1100 mg/kg, 1200 mg/kg, 1300 mg/kg, 1400 mg/kg, 1500 mg/kg, 1600 mg/kg, 1700 mg/kg, 1800 mg/kg, 1900 mg/kg, 2000 mg/kg of a compound as described herein. Useful pharmaceutically effective combinations can contain between about 300 mg/kg and about 1000 mg/kg of a compound as described herein, such as an FZD5 receptor blocker or a long chain fatty acid. For example, embodiments as described herein can comprise about 300 mg/kg of a compound.

Pharmaceutically effective combinations, such as a pill or tablet, can be comprise between 0.1 and 2000 mg of an FZD5 receptor blocker and/or a long chain fatty acid. For example, pharmaceutically effective combinations can comprise about 0.1 mg, 1 mg, 10 mg, 20 mg, 30 mg, 40 mg, 50 mg, 60 mg, 70 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg of a compound as described herein. Useful pharmaceutically effective combinations can contain between about 300 mg and about 1000 mg of an FZD5 receptor blocker and/or a long chain fatty acid. For example, embodiments as described herein can comprise about 300 mg of a compound as described herein.

Single unit dosage forms of the disclosure are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal (e.g., cream, lotion, or dermal spray) or transcutaneous administration to a subject. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions or solutions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms for parenteral administration to a subject.

The composition, shape, and type of dosage forms of the disclosure will vary depending on their use. Further, the dosage can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect to be achieved; and rate of excretion.

For example, a dosage form used in the acute treatment of a disease can contain larger amounts of one or more of the active agents it comprises than a dosage form used in the chronic treatment of the same disease. Similarly, a parenteral dosage form can contain smaller amounts of one or more of the active agents it comprises than an oral dosage form used to treat the same disease. These and other ways in which specific dosage forms encompassed by this disclosure will vary from one another will be readily apparent to those skilled in the art. See, e.g., Remington's Pharmaceutical Sciences, 18th ed., Mack Publishing, Easton Pa. (1990).

The invention also comprises the formation of pharmaceutically acceptable, stable salts of the compounds as described herein with metals or amines. Non-limiting examples of metals used as cations comprise alkali metals such as Na+ or K+ and alkaline-earth metals such as Mg2+ and Ca2+. Non-limiting examples of amines comprise N,N-dibenzylethylenediamine, chloro-procaine, choline, diethanolamine, ethylenediamine, N-methylglucamine and procaine.

A pharmaceutical composition of the invention is formulated to be compatible with its intended route of administration. Examples of routes of administration are described herein, and comprise parenteral, e.g., intravenous, intradermal, subcutaneous, oral (e.g., inhalation), transdermal (topical), transmucosal, nasal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

As an exemplary embodiment, pharmaceutical combinations of the invention can be administered orally, such as in the form of tablets containing excipients such as starch or lactose, or in capsules, such as alone or mixed with excipients, or in the form of syrups or suspensions containing coloring or flavoring agents. They can also be injected parenterally, for example intramuscularly, intravenously or subcutaneously. In parenteral administration, they can be used in the form of a sterile aqueous solution which can contain other solutes, such as, for example, any salt or glucose in order to make the solution isotonic.

The formulations can be administered to a subject for the treatment of cancer or an inflammatory condition, for example orally, such as covered in gelatin capsules or compressed in lozenges. For oral therapeutic administration, said compounds can be mixed with excipients and used in the form of lozenges, tablets, capsules, elixirs, suspensions, syrups, wafers, chewing gum, and the like. These preparations can contain at least 0.5% of active compound, but can vary depending on each form, such as between 4% and 75% approximately of the weight of each unit. The amount of active compound in such compositions can be that which is necessary for obtaining the corresponding dosage. For example, the compositions and preparations as described herein can be prepared in such a way that each oral dosage unit can contain between 0.1 mg and 300 mg of the active compound.

In parenteral therapeutic administration, the active compounds of this invention can be incorporated in a solution or suspension. Such preparations, for example, can contain at least 0.1% of the active compound, but can vary between 0.5% and 50% approximately of the weight of the preparation. For example, such preparations comprise about 0.1%, 0.5%, 1%, 5%, 10%, 15%, 25%, 30%, 35%, 40%, 45%, 50%, of the weight of the preparation. The amount of active compound in such compositions can be that which is necessary for obtaining the corresponding dosage. The compositions and preparations as described herein can be prepared in such a way that each parenteral dosage unit can contain between 0.01 mg and 1000 mg, for example between about 0.5 mg and 100 mg of the active compound, for example. While intramuscular administration can be given in a single dose or divided into up to multiple doses, such as three doses, intravenous administration can include a drip device for giving the dose by venoclysis. Parenteral administration can be performed by means of ampoules, disposable syringes or multiple-dose vials made of glass or plastic.

Pharmaceutical compositions or formulations suitable for injectable use can include sterile aqueous solutions (where water soluble) or dispersions and sterile powders for the extemporaneous preparation of sterile injectable solutions or dispersions. For intravenous administration, suitable carriers can include physiological saline, bacteriostatic water, Cremophor EM™ (BASF, Parsippany, N.J.) or phosphate buffered saline (PBS). In embodiments, the composition can be sterile and can be fluid to the extent that easy syringability exists. It can be stable under the conditions of manufacture and storage and can be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyethylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can occur by including an agent in the composition which delays absorption, for example, aluminum monostearate and gelatin.

Sterile injectable solutions can be prepared by incorporating the compound in the required amount in an appropriate solvent with one or a combination of ingredients enumerated herein, as required, followed by filtered sterilization. Dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional ingredient from a previously sterile-filtered solution thereof.

Oral compositions include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art.

The pharmaceutical formulations and compositions can be administered to a subject in a single dose for the treatment of a condition characterized by neuronal injury or neuronal damage, or as multiple doses over a period of time. Further, the formulation can be administered at intervals of about 4 hours, 8 hours, 12 hours, 24 hours, or longer. In embodiments, the formulation can be administered continuously over a period of time, such as for 4 hours, 8 hours, 12 hours, 24 hours, or longer.

Of necessity, there will be variations which will depend on the weight and conditions of the subject to be treated and on the administration route selected.

Pharmaceutical compositions described herein can be formulated as controlled-release pharmaceutical products, which have a goal of improving drug therapy over that achieved by their non-controlled counterparts. The use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

Methods of Detecting and Treating

Aspects of the invention are directed towards methods of treating a condition in a patient, wherein the condition is characterized by neuronal injury or neuronal damage. For example, embodiments comprise administering to a subject an effective amount of a pharmaceutical formulation or composition as described herein for the treatment of the condition.

In embodiments, the subject is administered an FZD5 receptor blocker or antagonist alone for the treatment of a condition. In embodiments, the subject is administered an FZD5 receptor blocker or antagonist together with a long chain fatty acid, such as a docosanoid. For example, docosanoids such as NPD1 potentiate effects of FZD5 receptor blocker in vitro.

“Neuronal injury” or “neuronal damage” can refer to the damage to the function or structure (e.g., cytoskeletal damage) of neurons as a result of an insult or trauma to the nervous system, or certain diseases. Neuronal injury is associated with, for instance: stroke, ischemic events (e.g., brain ischemia, ischemia of the eyes), seizures of diverse etiology (epileptic, associated with brain injury, of genetic origin), spinal cord injury or trauma, brain damage due to drugs of abuse, or excitotoxic insults of diverse nature.

In embodiments, the “neuronal injury” or “neuronal cell death” can be the result of a stroke. For example, “stroke” can refer to any acute, clinical event related to the impairment of cerebral circulation. The terms “acute cerebral ischemia” and “stroke” can be used interchangeably. As described herein, neuronal damage in a stroke model was significantly decreased by the administration of Box5 and DHA in tissue and behavioral studies.

In other embodiments, neuronal injury or neuronal damage can be the result of conditions such as retinal damage, Alzheimer's disease, traumatic brain injury (TBI), Parkinson's Disease, or age-related macular degeneration (AMD); Diabetic: retinopathy, glomerulonephritis and neuropathy; Psoriatic-arthritis, Crohn's disease, Rheumatoid-arthritis, Atherosclerosis, asthma, periodontitis, Hashimoto thyroiditis, Systemic lupus erythematosus, Multiple sclerosis, fibromyalgia and metabolic syndrome.

In embodiments, the neuronal damage or neuronal injury is the result of or exacerbated by uncompensated oxidative stress

As used herein, “neurodegeneration” can refer to the progressive loss of individual or collective structure or function of neurons, up to and including the death of neurons that is associated with many neurodegenerative diseases.

For example, “neurodegenerative disease(s)” or “neurodegenerative disorder” can refer to medical conditions that are characterized clinically by their insidious onset and chronic progression. In many instances, parts of the brain, spinal cord, or peripheral nerves functionally fail and the neurons of the dysfunctional region die. Neuroanatomically localizable functional impairment and “neurodegeneration” associate with recognizable syndromes or conditions that are ideally distinct, although in clinical and even neuropathologic practice substantial overlap exists. Neurodegenerative diseases are often categorized by whether they initially affect cognition, movement, strength, coordination, sensation, or autonomic control.

Frequently, however, patients will with symptoms and signs referable to more than one system. Involvement of several systems can occur concomitantly, or else by the time the patient has functionally declined enough to seek medical attention multiple systems have become involved. In many cases, the diagnosis of a neurodegenerative disease cannot be critically ‘confirmed’ by a simple test.

The term “neurodegenerative” can refer to the loss of neurons that cause disease.

However, without being bound by theory, neuronal demise can be the final stage of a preceding period of neuron dysfunction. It is difficult to know whether clinical decline associates with actual neuron loss, or with a period of neuron dysfunction that precedes neuron loss. Also, neurodegenerative diseases are etiologically heterogeneous. In addition to syndromically defining neurodegenerative diseases by what neuro-anatomical system is involved, these disorders are broken down along other clinical lines. Early (childhood, young adulthood, or middle aged adulthood) versus late (old age) onset is an important distinction. Some clinically similar neurodegenerative diseases are sub-categorized by their age of onset, despite the fact that at the molecular level different forms of a disease can have very little in common. Sporadic onset versus Mendelian (genetic) inheritance constitutes another important distinction, and many named neurodegenerative diseases have both sporadic (wherein Mendelian inheritance is not recognizable) and Mendelian subtypes.

Non-limiting examples of neurodegenerative diseases comprise: dementia, for example Alzheimer's Disease, multi-infarct dementia, AIDS-related dementia, and Fronto temporal Dementia; neurodegeneration associated with cerebral trauma; Parkinson's Disease; Amyotrophic Lateral Sclerosis (ALS); Multiple Sclerosis (MS); Huntington's disease; neurodegeneration associated with stroke; neurodegeneration associated with cerebral infarct; hypoglycemia-induced neurodegeneration; neurodegeneration associated with epileptic seizure; neurodegeneration associated with neurotoxin poisoning; and multi-system atrophy.

Neurodegenerative diseases can present with memory loss or personality change, non-limiting examples of which comprise Alzheimer's disease, Frontotemporal Dementias, Dementia with Lewy Bodies, Prion diseases.

Neurodegenerative diseases can present as problems with movement, non-limiting examples of which comprise Parkinson's disease, Huntington's disease, Progressive Supranuclear Palsy, Corticobasal Degeneration, Multiple System Atrophy.

For example, neurodegenerative diseases can present as weakness, non-limiting examples of which comprise, amyotrophic lateral sclerosis, inclusion body myositis, degenerative myopathies.

Neurodegenerative diseases can also present as poor balance, non-limiting examples of which comprise the spinocerebellar atrophies.

Disorders of myelin include multiple sclerosis and Charcot-Marie-Tooth disease.

The term “motor neuron diseases” (MNDs) refers to a group of progressive neurological disorders that destroy motor neurons, the cells that control essential voluntary muscle activity such as speaking, walking, breathing, and swallowing. The best-known motor neuron disease is amyotrophic lateral sclerosis (ALS). Normally, messages from nerve cells in the brain (called upper motor neurons) are transmitted to nerve cells in the brain stem and spinal cord (called lower motor neurons) and from them to muscles. Upper motor neurons direct the lower motor neurons to produce movements such as walking or chewing. Lower motor neurons control movement in the arms, legs, chest, face, throat, and tongue. Spinal motor neurons are also called anterior horn cells. Upper motor neurons are also called corticospinal neurons.

When there are disruptions in the signals between the lowest motor neurons and the muscle, the muscles do not work properly; the muscles gradually weaken and can begin wasting away and develop uncontrollable twitching (called fasciculations). When there are disruptions in the signals between the upper motor neurons and the lower motor neurons, the limb muscles develop stiffness (called spasticity), movements become slow and effortful, and tendon reflexes such as knee and ankle jerks become overactive. Over time, the ability to control voluntary movement can be lost. The following is a list of the MNDs: Amyotrophic lateral sclerosis (ALS), also called Lou Gehrig's disease, progressive bulbar palsy, also called progressive bulbar atrophy, pseudobulbar palsy, Primary lateral sclerosis (PLS), progressive muscular atrophy, spinal muscular atrophy (SMA) and some of its variants (e.g., SMA type I, also called Werdnig-Hoffmann disease, SMA type II, SMA type III also called Kugelberg-Welander disease, congenital SMA with arthrogryposis, Kennedy's disease, also known as progressive spinobulbar muscular atrophy and post-polio syndrome (PPS)).

An “effective amount”, “sufficient amount” or “therapeutically effective amount” can refer to an amount sufficient to effect beneficial or clinical result, such as killing the cancerous cells, inhibiting the growth of the cancer, inhibiting the metastasis of the cancer, and/or reducing or inhibiting inflammation.

Pharmaceutical formulations as described herein can be administered to a subject by any suitable means, such as oral, intravenous, parenteral, subcutaneous, intrapulmonary, topical, intravitreal, dermal, transmucosal, rectal, and intranasal administration. Parenteral infusions include intramuscular, intravenous, intraarterial, or intraperitoneal administration. The formulations can also be administered transdermally, for example in the form of a slow-release subcutaneous implant or as a transdermal patch. They can also be administered by inhalation. Although direct oral administration can cause some loss of activity, the compounds can be packaged in such a way to protect the active ingredient(s) from digestion by use of enteric coatings, capsules or other methods known in the art.

In methods described herein, the pharmaceutical formulation can be administered to the subject one time (e.g., as a single injection or deposition). Alternatively, administration can be once or twice daily to a subject in need thereof for a period of from about 2 to about 28 days, or from about 7 to about 10 days, or from about 7 to about 15 days. It can also be administered once or twice daily to a subject for a period of 1, 2, 3, 4, 5, 6, 7, 8, 9, 10, 11, 12 times per year, or a combination thereof.

Any of the therapeutic applications described herein can be applied to any subject in need of such therapy, including, for example, a mammal such as a mouse, a rat, a dog, a cat, a cow, a horse, a rabbit, a monkey, a pig, a sheep, a goat, or a human. In some embodiments, the subject is a mouse, rat, pig, or human. In some embodiments, the subject is a mouse. In some embodiments, the subject is a rat. In some embodiments, the subject is a pig. In some embodiments, the subject is a human.

Embodiments can comprise the steps of obtaining a sample from a patient suffering from a condition, such as a neurodegenerative disease; measuring the protein level of Wnt5a protein in the sample and comparing the protein level of Wnt5a protein in the sample to a control sample; and treating the patient, such as with a composition or formulation described herein. Further, embodiments can comprise the step of diagnosing the patient as having a neurodegenerative disease if the protein level of Wnt5a protein in the sample is higher than that of the control sample.

Aspects of the invention are also directed towards methods for determining the prognosis of a patient suffering from a neurodegenerative disease. For example, such methods comprise, comprising obtaining a sample from a patient suffering from a neurodegenerative disease; measuring the expression level of Wnt5a protein in the sample and comparing the expression level of Wnt5a protein in the sample to a control sample (such as a sample from a normal individual and/or an unaffected individual); and determining the prognosis of the patient. The patient can be administered a composition or formulation as described herein.

Aspects of the invention comprise measuring or detecting in a biological sample a biomarker of a disease or condition characterized by neuronal injury, neurodegeneration, or neuronal damage. The term “biomarker” can refer to a nucleic acid, a protein, a part of a nucleic acid or protein, a peptide or a polypeptide, which can be used as a biological marker, e.g. for prophylactic, diagnostic, or therapeutic purposes. See, for example, U.S. Pat. Nos. 10,443,099; 10,442,862; 10,435,756; 10,431,326; 10,428,385; 10,407,730; 10,392,667; or 10,378,066, each of which are incorporated herein by reference in their entireties.

Biomarkers of the invention can be measured in different types of biological samples. Non-limiting examples of biological samples that can be used in methods of the invention, although not intended to be limiting, include tissue biopsy, stool, plasma, cord blood, neonatal blood, cerebral spinal fluid, tears, vomit, saliva, urine, feces, and meconium. A sample can be prepared to enhance detectability of the biomarkers. For example, a sample from the subject can be fractionated. Any method that enriches for a biomarker polypeptide of interest can be used. Sample preparations, such as prefractionation protocols, are optional and may not be necessary to enhance detectability of biomarkers depending on the methods of detection used. For example, sample preparation can be unnecessary if an antibody that specifically binds a biomarker is used to detect the presence of the biomarker in a sample. Sample preparation can involve fractionation of a sample and collection of fractions determined to contain the biomarkers. Methods of prefractionation include, for example, size exclusion chromatography, ion exchange chromatography, heparin chromatography, affinity chromatography, sequential extraction, gel electrophoresis and liquid chromatography.

Aspects of the invention comprise an assay that measures the expression level of Wnt5a protein. For example, such as measuring or detecting Wnt5a protein using assays known to the art, such as Western blot; ELISA (enzyme linked immunosorbent assay); radioimmunoassay analysis (RIA); radial immunodiffusion; Ouchterlony immunodiffusion; rocket immunoelectrophoresis; tissue immunohistochemistry; immunoprecipitation assays; complement fixation assays; flow cytometry; protein chip (protein microarray); capillary western blot; protein MS; protein sequencing; HPLC; and gas chromatography. Additional, non-limiting examples of assays include an immunoassay, a colorimetric assay, fluorimetric assay or a combination thereof. Non-limiting examples of immunoassays comprise a Western blot assay, an enzyme-linked immunosorbent assay (ELISA), immunoprecipitation or a combination thereof, among those described herein. For example, a biological sample collected from a subject can be incubated together with a biomarker specific antibody, such as an anti-Wnt5a antibody or fragment thereof, and the binding of the antibody to the biomarker in the sample is detected or measured. Wnt5a can be measured at one or more intervals, such as at intervals after the onset of disease symptoms or at intervals after the onset of treatment. In an individual afflicted with a stroke, for example, Wnt5a expression levels can be measured within seven days, five days or three days after the stroke event, and can then be measured at intervals after the initial measurement.

In embodiments, the sample to be tested is compared to a control sample. As used herein, “changed as compared to a control” sample or subject is understood as having a level of the analyte or diagnostic or therapeutic indicator (e.g., biomarker such as Wnt5a) to be detected at a level that is statistically different than a sample from a normal, untreated, or abnormal state control sample. Determination of statistical significance is within the ability of those skilled in the art, e.g., the number of standard deviations from the mean that constitute a positive or negative result and the statistical analyses to arrive at these intervals.

In embodiments, the antibody or fragment thereof can be specific for Wnt5a (anti-Wnt5a). The antibody can be a polyclonal antibody or a monoclonal antibody. The antibody or fragment thereof can be attached to a molecule which can be identified, visualized or localized using known methods. Suitable detectable labels include radioisotopic labels, enzyme labels, non-radioactive isotopic labels, fluorescent labels, toxin labels, affinity labels, and chemiluminescent labels.

Examples of assays that can be used in methods of the invention, although not intended to be limiting, comprise a Bradford assay, a bicinchoninic acid (BCA) assay, a Lowry assay, a pyrogallol red protein dye-binding assay, a Coomassie blue dye-binding assay, an endpoint assay, a kinetic assay, such as a kinetic assay using a fluorometric substrate such as 4-methyllumbelliferyl phosphate, chemiluminescent substrates such as CSPD and CDP-Star, DynaLight Substrate with RapidGlow enhancer, or colorimetric 4-nitrophenyl phosphate, an assay to detect phosphatase reactions, an assay to detect ATP hydrolysis, or a combination thereof. In embodiments, the assays can be provided in a multiwall format, such as a 6-, 12-, 24-, 48, or 96-well plate. In embodiments, the assays can be provided in a standard cuvette, such as a 1 ml cuvette.

The enzyme employed in embodiments herein, for example to detect protein levels or enzymatic activity, can be, for example, alkaline phosphatase, horseradish peroxidase, β-galactosidase and/or glucose oxidase; and the substrate can respectively be an alkaline phosphatase, horseradish peroxidase, β-galactosidase or glucose oxidase substrate (see Molecular Probes Handbook—A Guide to Fluorescent Probes and Labeling Technologies, 11th Edition (2010), Invitrogen, which is incorporated by reference herein in its entirety).

In embodiments, the enzyme, such as alkaline phosphatase or horseradish peroxidase, can be attached to a secondary antibody.

Alkaline phosphatase (AP) substrates include, but are not limited to, AP-Blue substrate (blue precipitate, Zymed catalog p. 61); AP-Orange substrate (orange, precipitate, Zymed), AP-Red substrate (red, red precipitate, Zymed), 5-bromo, 4-chloro, 3-indolyphosphate (BCIP substrate, turquoise precipitate), 5-bromo, 4-chloro, 3-indolyl phosphate/nitroblue tetrazolium/iodonitrotetrazolium (BCIP/INT substrate, yellow-brown precipitate, Biomeda), 5-bromo, 4-chloro, 3-indolyphosphate/nitroblue tetrazolium (BCIP/NBT substrate, blue/purple), 5-bromo, 4-chloro, 3-indolyl phosphate/nitroblue tetrazolium/iodonitrotetrazolium (BCIP/NBT/INT, brown precipitate, DAKO, Fast Red (Red), Magenta-phos (magenta), Naphthol AS-BI-phosphate (NABP)/Fast Red TR (Red), Naphthol AS-BI-phosphate (NABP)/New Fuchsin (Red), Naphthol AS-MX-phosphate (NAMP)/New Fuchsin (Red), New Fuchsin AP substrate (red), p-Nitrophenyl phosphate (PNPP, Yellow, water soluble), VECTOR™ Black (black), VECTOR™ Blue (blue), VECTOR™ Red (red), Vega Red (raspberry red color).

Horseradish Peroxidase (HRP, sometimes abbreviated PO) substrates include, but are not limited to, 2,2′ Azino-di-3-ethylbenz-thiazoline sulfonate (ABTS, green, water soluble), aminoethyl carbazole, 3-amino, 9-ethylcarbazole AEC (3A9EC, red). Alpha-naphthol pyronin (red), 4-chloro-1-naphthol (4C1N, blue, blue-black), 3,3′-diaminobenzidine tetrahydrochloride (DAB, brown), ortho-dianisidine (green), o-phenylene diamine (OPD, brown, water soluble), TACS Blue (blue), TACS Red (red), 3,3′,5,5′ Tetramethylbenzidine (TMB, green or green/blue), TRUE BLUE™ (blue), VECTOR™ VIP (purple), VECTOR™ SG (smoky blue-gray), and Zymed Blue HRP substrate (vivid blue).

Glucose Oxidase (GO) substrates, include, but are not limited to, nitroblue tetrazolium (NBT, purple precipitate), tetranitroblue tetrazolium (TNBT, black precipitate), 2-(4-iodophenyl)-5-(4-nitrophenyl)-3-phenyltetrazolium chloride (INT, red or orange precipitate), Tetrazolium blue (blue), Nitrotetrazolium violet (violet), and 3-(4,5-dimethylthiazol-2-yl)-2,5-diphenyltetrazolium bromide (MTT, purple). All tetrazolium substrates require glucose as a co-substrate. The glucose gets oxidized and the tetrazolium salt gets reduced and forms an insoluble formazan which forms the color precipitate.

Beta-Galactosidase substrates, include, but are not limited to, 5-bromo-4-chloro-3-indoyl beta-D-galactopyranoside (X-gal, blue precipitate).

Other examples of alkaline and acid phosphatase substrates comprise 9H-(1,3-dichloro-9,9-dimethylacridin-2-one-7-yl) phosphate, diammonium salt (DDAO phosphate), 6,8-difluoro-4-methylumbelliferyl phosphate (DiFUP), fluorescein diphosphate, tetraammonium salt (FDP), 4-methylumbelliferyl phosphate, free acid (MUP), and 4-methylumbelliferyl phosphate, dicyclohexylammonium salt, trihydrate (MUP DCA salt).

Alkaline phosphatase activity, such as intestinal alkaline phosphatase activity, can be detected and/or measured with use of chromogenic substrates and/or fluorogenic substrates of alkaline phosphatases. For example, 4-methylumbelliferyl phosphate (UP) is a fluorogenic substrate for alkaline phosphatases, and alkaline phosphatase mediated hydrolysis of its phosphate substituent yields the blue-fluorescent 4-methylumbelliferyl (excitation/emission ˜386/448 nm). In embodiments, the alkaline phosphatase substrate can be directly admixed with the biological sample, such as stool, allowing for the direct detection of the presence of alkaline phosphatase or the measurement of its activity.

Alkaline phosphatase (AP) substrates include, but are not limited to, AP-Blue substrate (blue precipitate, Zymed catalog p. 61); AP-Orange substrate (orange, precipitate, Zymed), AP-Red substrate (red, red precipitate, Zymed), 5-bromo, 4-chloro, 3-indolyphosphate (BCIP substrate, turquoise precipitate), 5-bromo, 4-chloro, 3-indolyl phosphate/nitroblue tetrazolium/iodonitrotetrazolium (BCIP/INT substrate, yellow-brown precipitate, Biomeda), 5-bromo, 4-chloro, 3-indolyphosphate/nitroblue tetrazolium (BCIP/NBT substrate, blue/purple), 5-bromo, 4-chloro, 3-indolyl phosphate/nitroblue tetrazolium/iodonitrotetrazolium (BCIP/NBT/INT, brown precipitate, DAKO, Fast Red (Red), Magenta-phos (magenta), Naphthol AS-BI-phosphate (NABP)/Fast Red TR (Red), Naphthol AS-BI-phosphate (NABP)/New Fuchsin (Red), Naphthol AS-MX-phosphate (NAMP)/New Fuchsin (Red), New Fuchsin AP substrate (red), p-Nitrophenyl phosphate (PNPP, Yellow, water soluble), VECTOR™ Black (black), VECTOR™ Blue (blue), VECTOR™ Red (red), Vega Red (raspberry red color).

Other substrates known in the art, including those described herein, can be used with embodiments of the invention (see Molecular Probes Handbook—A Guide to Fluorescent Probes and Labeling Technologies, 11th Edition (2010), Invitrogen, which is incorporated by reference herein in its entirety). Further, various fluorophores known in the art can be covalently attached to the substrate, such as MUP.

Enzyme reactions can provide a highly specific, rapid and sensitive assay for detection of specific proteins in a sample, such as iAP in stool. Examples of suitable fluorogenic substrates which can be utilized within the invention comprise Fluorescein diacetate, 4-Methylumbelliferyl acetate, 4-Methylumbelliferyl casein, 4-Methylumbelliferyl-α-L-arabinopyranoside, 4-Methylumbelliferyl-β-D-fucopyranoside, 4-Methylumbelliferyl-α-L-fucopyranoside, 4-Methylumbelliferyl-β-L-fucopyranoside, 4-Methylumbelliferyl-α-D-galactopyranoside, 4-Methylumbelliferyl-β-D-galactopyranoside, 4-Methylumbelliferyl-α-D-glucopyranoside, 4-Methylumbelliferyl-β-D-glucopyranoside, 4-Methylumbelliferyl-β-D-glucuronide, 4-Methylumbelliferyl nonanoate, 4-Methylumbelliferyl oleate, 4-Methylumbelliferyl phosphate, bis(4-Methylumbelliferyl)phosphate, 4-Methylumbelliferyl pyrophosphate diester, 4-Methylumbelliferyl-β-D-xylopyranoside.

Non-limiting examples of suitable chromogenic substrates for use within the invention comprise o-Nitrophenyl-β-D-galactopyranoside, p-Nitrophenyl-β-D-galactopyranoside, o-Nitrophenyl-β-D-glucopyranoside, p-Nitrophenyl-α-D-glucopyranoside, p-Nitrophenyl-β-D-glucopyranoside, p-Nitrophenyl-β-D-glucuronide, p-Nitrophenyl phosphate, o-Nitrophenyl-β-D-xylopyranoside, p-Nitrophenyl-α-D-xylopyranoside, p-Nitrophenyl-β-D-xylopyranoside, and Phenolphthalein-β-D-glucuronide.

Medical Kits

Aspects of the invention are directed towards a medical kit suitable for the treatment of a condition characterized by neuronal injury or neuronal damage. In embodiments, the kit can comprise printed instructions for administering a formulation or composition as described herein to a subject in need thereof; a pharmaceutical composition as described herein, and/or a pharmaceutically acceptable carrier. A “kit” or “medical kit” of the disclosure comprises a dosage form of a formulation of the disclosure or a pharmaceutically acceptable salt, solvate, hydrate, stereoisomer, prodrug, or clathrate thereof. A kit can also include two or more compounds as described herein, such as in combination, such as in a single tablet, or provided separately, such as in two or more tablets.

Kits can further comprise additional active agents, examples of which are described herein. Kits of the disclosure can further comprise devices that are used to administer the active ingredients. Examples of such devices include, but are not limited to, syringes, drip bags, patches, and inhalers. Kits can also comprise printed instructions for administering the formulation to a subject.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Aspects of the invention are further directed towards diagnostic kits for determining brain injury status in a patient. For example, such a diagnostic kit can comprise a substrate for collecting a sample from the patient; and means for measuring the protein level of Wnt5a protein. Exemplary, albeit non-limiting, means for measuring Wnt5a comprises Western blot; ELISA (enzyme linked immunosorbent assay); radioimmunoassay analysis (RIA); radial immunodiffusion; Ouchterlony immunodiffusion; rocket immunoelectrophoresis; tissue immunohistochemistry; immunoprecipitation assays; complement fixation assays; flow cytometry; protein chip (protein microarray); capillary western blot; protein MS; Protein sequencing; HPLC; and gas chromatography, among those described herein.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Underscoring aspects of the invention, Wnt5a mainly by a noncanonical Wnt signaling (beta-catenin independent signaling) induces brain and retina damage. Wnt5a/Frizzled 5 receptor trigger inflammatory responses via NFkB/p65 and cell damage in retinal pigment epithelial (RPE) cells undergoing uncompensated oxidative stress (UOS) and in ischemic stroke. Docosahexaenoic acid (DHA) and its bioactive derivative, Neuroprotectin D1, upregulates c-Rel expression, which in turn blunts Wnt5a abundance in RPE cells by competing with NF-kB for specific sites in the Wnt5a promoter A. For example, in an embodiment, Box5 plus docosanoid combinatorial therapy can be used for treatment of ischemic stroke, retina damage and neurodegenerative diseases, including Alzheimer's Disease (AD), TBI, Parkinson's Disease and age-related macular degeneration, such as of the dry form.

Experiments uncovered a new participant in the setting in motion inflammatory signals occurring in retina and brain at the onset of damage (Wnt5a). We discovered that BOX5 (at an upstream site) and DHA (at a downstream site) blunt damage, eliciting remarkable neuroprotection evidenced by MRI and neurological recovery. Wnt5a is decreased in AD. The targets and mechanism are advantageous and allow the application of new therapeutic avenues to target onset and early progression of brain and retina damage that include neurodegenerative diseases. In addition, these therapies can also be applied to atherosclerosis, sepsis, rheumatoid arthritis, and psoriasis vulgaris, obesity and type-2 diabetes.

### Example 2

### INTRODUCTION

Wnt signaling pathways are associated with function and pathology, including development and cancer (Nusse and Clevers, 2017). Wnt5a from the wingless family of ligands is a secretory lipid-modified glycoprotein that activates a non-canonical calcium-dependent signaling via interaction with Frizzled proteins, Ror1/2, RYK, and RTK (De, 2011). Wnt5a activity is related to the cellular milieu context and to the availability of receptors and co-receptors, and can also activate beta-catenin via promiscuous interaction with LRP5/6 (Ring et al. 2014). Wnt5a is engaged in cell fate, differentiation, and other processes. Recently, Wnt5a has been associated with inflammatory diseases, like rheumatoid arthritis (Sen et al. 2001; Rauner et al. 2012) and atherosclerosis (Ackers et al. 2015). Moreover, Wnt5a is released by macrophages (Naskar et al. 2014; Pereira et al. 2008) to activate NF-κB via FZD5 and ROR2 (Zhao et al. 2014). Because NF-κB/p65 is a Wnt5a transcriptional activator (Katula et al. 2012), Wnt5a boosts its own expression and release in a positive loop.

Thus, we asked whether DHA/NPD1, modulators of uncompensated oxidative stress (UOS) and cell survival (Mukherjee et al. 2004; Calandria et al. 2012) regulates Wnt5a expression and its extracellular availability. Using human primary retinal pigment epithelial (hpRPE) cells, we have addressed events relevant to retinal degenerations, since these cells support photoreceptor integrity, by NPD1 synthesis from DHA (Calandria et al. 2009; Bazan, 2007; Bazan, 2006).

The disclosure shows that cREL mediates Wnt5a transcriptional regulation in these cells by NPD1 and that this lipid mediator enhances the internalization of Wnt5a/FZD5 leading to Wnt5a degradation via clathrin-dependent endocytosis. In brain ischemia-reperfusion, DHA fosters neuron survival via NPD1 synthesis that in turn activates NF-KB/cRel (Calandria et al. 2015). This disclosure further shows that Wnt5a is upregulated in stroke penumbra and augmented in the bloodstream, favoring activation of immune cells and their recruitment into damaged tissue. In additional embodiments, DHA decreased bloodstream and penumbra Wnt5a abundance, leading to neuroprotection. This disclosure identifies that DHA/NPD1 inhibits UOS-enhanced Wnt5a transcription and secretion in human RPE cells. Also, the disclosure shows that Frizzled 5 (FZD5) activates inflammatory components of NF-κB, which heightens Wnt5a transcription. Likewise, DHA/NPD1 after ischemic stroke decreased brain NF-κB/p65-driven Wnt5a transcription, interleukin-1 beta (IL1β), tumor necrosis factor alpha (TNFα) and other pro-inflammatory genes expression promoting neuronal survival. Altogether, these data indicate an inflammatory modulatory signaling by DHA/NPD1 engaging Wnt5a in responses to neural cell injury.

In certain embodiments, the disclosed biomarker and therapeutics are applied to treat atherosclerosis, sepsis, rheumatoid arthritis, psoriasis vulgaris, obesity, type-2 diabetes, a combination thereof, or any other disease or disorder involving inflammation. Embodiments of the disclosed invention treat the onset and early progression of cellular damage. Cellular damage can include, but is not limited to brain damage, retina damage, or other neuronal cell damage.

Docosanoids Inhibits UOS-Triggered Wnt5a and FZD5 Transcription with Concomitant Reduction in Apoptosis.

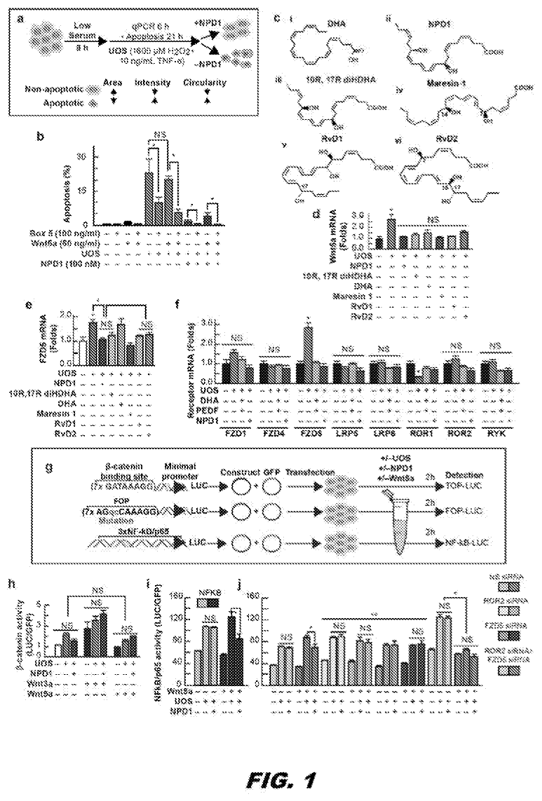

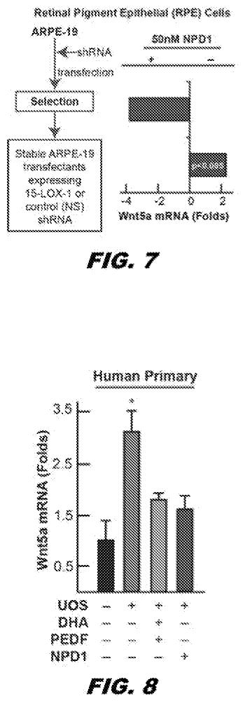

UOS by H₂O₂ plus TNFα triggers NPD1 synthesis via 15-lipoxygenase-1 (15-LOX-1) in RPE cells (Calandria et al. 2009) and silencing of this enzyme induces NPD1 depletion. Under UOS, 15-LOX-1 deficient cells display a 2-fold increase in Wnt5a expression (FIG. 7) that was brought down to below controls by NPD1. Also, DHA plus pigment epithelium-derived factor (PEDF), a neurothrophin agonist of NPD1 synthesis (Mukherjee et al. 2007), prevented Wnt5a up-regulation in hpRPE cells (FIG. 8). NPD1 and other docosanoids (FIG. 1C i-vi) down regulated Wnt5a transcription as well (FIG. 1D).

Recombinant Wnt5a potentiated cell death by UOS in ARPE-19 cells (FIG. 9) and in hpRPE (FIG. 1B). Box5, a specific inhibitor of Wnt5a binding to FZD5 (Jenei et al. 2009), or NPD1, blocked apoptosis by UOS in the presence of the Wnt ligand. Box5 also decreased apoptosis in cells only exposed to UOS, indicating that endogenous Wnt5a is involved in this cellular damage. Wnt5a alone had no effect on RPE cells, denoting that the Wnt ligand enhance apoptosis in susceptible cells undergoing UOS but not in resting cells.

To assess the involvement of Wnt5a receptors/co-receptors (FZD4 and 5, LRP5/6, RYK, and ROR1/2) (Mikels and Nusse, 2006), we measured their expression by SYBR green-based real-time PCR. A 3-fold increase in FZD5 expression took place, and NPD1 counteracted this effect (FIG. 1F). FZD1, FZD4, LRP5, LRP6, ROR2, and RYK, remained unchanged, although ROR1 expression was reduced under UOS and re-established by NPD1 (FIG. 1F). When hpRPE were exposed to UOS, FZD5 mRNA rose 2-fold and then dropped to control levels when docosanoids were added (FIG. 1E). DHA is the precursor of 10R, 17R diHDHA, Maresin 1, RVD1, RVD2, and NPD1 (FIG. 1C i-vi). However, DHA alone did not affect the expression of FZD5, indicating that its conversion to lipid mediators is required to counteract the effect of UOS on FZD5. These results also indicate that FZD5 is linked to Wnt5a signaling in RPE cells undergoing UOS.

Wnt5a-Dependent Activation of NF-κB Requires FZD5 and ROR2.

Wnt5a have dual effects on P-catenin activity, positive and negative, depending on the receptor context (Mikels and Nusse, 2006). We tested whether or not Wnt5a activates p3-catenin using TOP Flash/FOP flash reporter system in hpRPE cells undergoing UOS+/−NPD1 (FIG. 1g). In the absence of Wnt5a or Wnt3a, β-catenin activity was not altered by UOS or NPD1 (FIG. 1H). When Wnt3a was added, luciferase rose almost twice consequently with β-catenin activation via Wnt canonical pathway. Wnt5a alone did not affect β-catenin (FIG. 1H), indicating that Wnt5a signaling in the absence of activated p-catenin does not involve TCF/LEF-related gene expression.

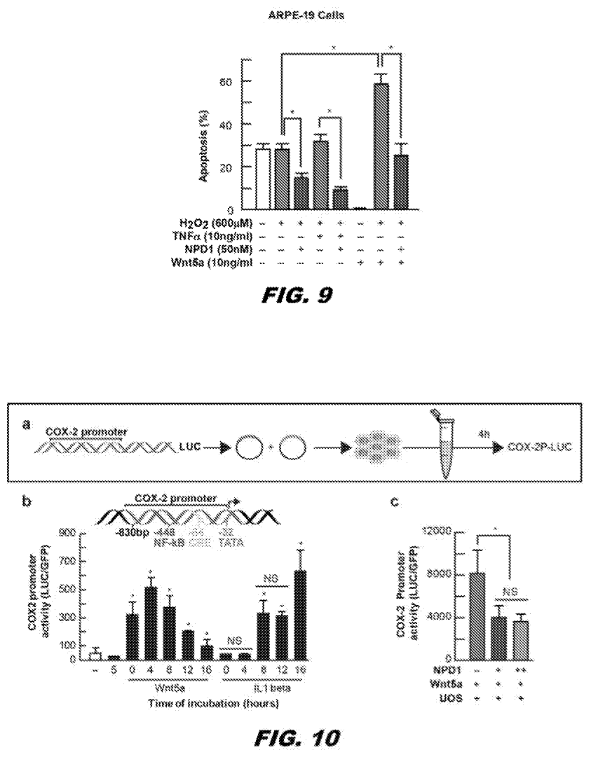

Since Wnt5a induces COX-2 transcription (Kim et al. 2010), NF-κB and other factors can be involved in its response (Onodera et al. 2015). We asked if Wnt5a activates inflammatory signaling to enhance apoptosis mediated by UOS by assessing COX-2 promoter activation (FIG. 10). Recombinant Wnt5a (rWnt5a) induced COX-2 promoter activation (FIG. 10A). The COX-2 promoter activity reached its peak 4 hours after adding rWnt5a (FIG. 10B). To compare Wnt5a effects with other cytokines that enhance COX-2 expression, Interleukin-1β (IL-1β) activity was measured. IL-1 β showed a growing staggered pattern of COX-2 promoter activation through at least 16 hours (FIG. 10B). Wnt5a also increased COX-2 activation in hpRPE indicating that it is activating NF-κB in RPE cells. NPD1 was sufficient to bring promoter activity down by half (FIG. 10C). Wnt5a interacts with FZD5 and ROR2 to trigger inflammatory gene expression via NF-κB activation (Naskar et al. 2014; Sato et al. 2015). To determine if Wnt5a activates NF-κB, hpRPE were transfected with a construct that encompassed three p65 high-affinity binding sites in tandem, driving the expression of the luciferase reporter gene (FIG. 1G). RPE cells exposed to UOS showed increased luciferase activity, and NPD1 did not affect NF-κB/p65 as shown previously (FIG. 11) (Calandria et al. 2015). Wnt5a heightens NF-κB activation, and the NPD1 reduced luciferase activity (FIG. 1I). To assess whether or not FDZ5 and ROR2 were involved in NF-κB activation by Wnt5a, cells were co-transfected with siRNAs targeting the two receptors (FIG. 11). Cells, co-transfected with control siRNA, showed the same pattern of NF-κB activity seen in FIG. 11. ROR2 or FZD5 siRNAs separately abolished the difference between UOS and UOS+NPD1 (FIG. 1J). The co-transfection of both siRNAs, against ROR2 and FZD5 together, induced a higher NF-κB activation that was not affected by NPD1. When Wnt5a was added to the double knockdown cells, the NF-κB activity did not differ from controls in UOS or UOS-plus-NPD1 conditions indicate that the Wnt5a/FZD5/ROR2 competes with UOS on the activation of NF-κB, and NPD1 only targets the one triggered by Wnt5a.

NPD1 Induces cRel Binding to Wnt5a Promoter A that in Turn Prevents its Transcriptional Activation.

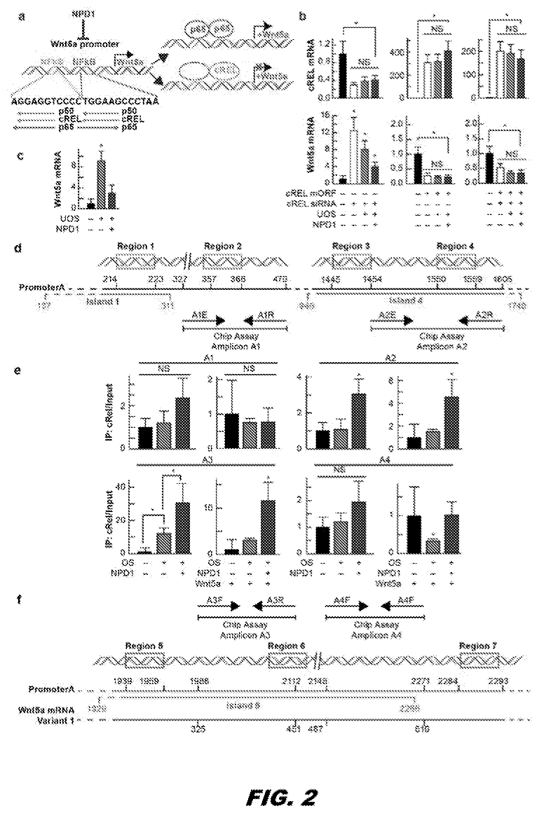

The Wnt5a gene is under the regulation of two promoters located in exon1a and exon2 (Vaidya et al. 2016). Promoter A drives the expression of the largest form, variant-1, and contains at least two binding sites for NF-κB (Katula et al. 2012). In-silico analysis using TRED software showed that both sites have a high affinity for three NF-κB members: p50, cRel, and p65. Downstream, p50 and cRel binding are opposed to p65 site, indicating that p65 and cRel activity compete to oppose each other (FIG. 2A and Table 4). To confirm a direct link between cRel expression and Wnt5a transcription, we over-expressed or silenced the transcription factor in hpRPE (FIG. 2B). cRel overexpression decreased Wnt5a mRNA, while the KO increased Wnt5a expression 12-fold in control, 8-fold in cells undergoing UOS, and 4-fold when NPD1 was added (FIG. 2B). The differences between treatments when cRel was silenced indicate an additional regulator of Wnt5a promoter A. When hpRPE were transfected with wild-type, or siRNA-resistant cREL ORF, along with the corresponding siRNA, cRel abundance was increased (FIG. 2B), and Wnt5a expression was decreased, indicating that there was no siRNA off-target effect on Wnt5a. The increase in cRel availability dominantly shut off Wnt5a expression regardless of the treatment. Altogether, the data indicate that cRel blocks Wnt5a expression triggered by UOS, which can be a key modulatory NPD1 function.

Based on the regions found to bind NF-κB members (Table 4), we designed four sets of primers that bound in the proximity or within the regions of interest to perform SYBR green-based real-time PCR detection (FIG. 2, D-F and Table 3). FIGS. 2, D and F and Table 5, also show regions of high probability for methylation, a mechanism for blocking Wnt5a transcription (Vaidya et al. 2016). Micrococcal DNase digested genomic DNA fragments from UOS or UOS-plus-NPD1-treated RPE cells, +/−rWnt5a, were pulled down by an antibody against cRel and used to test the four sets of primers. Amplicon A1, localized close to Region 1 and overlap to Region 2 cRel/p65 binding sites (FIG. 2D), showed no differences between treatments, indicating no differential binding of cRel to the genomic DNA in the presence of Wnt5a and NPD1 (FIG. 2E). Methylation of Region 1 may occur since it was predicted by the Methprimer software (Table 5). The amplicon 2 encompassing Region 3 and 4 displayed twice the amount of cRel bound to genomic DNA under NPD1 treatment. The amplicon 3, within the proximity of Region 5 and overlaps Region 6 (FIG. 2F), displayed the largest differences between treatments reaching more than 20-fold when NPD1 was added to RPE cells undergoing UOS in the absence of Wnt5a and 10-fold when Wnt5a was present. The results obtained for amplicon 3, indicate competition between the Wnt5a-activated p65 and NPD1-activated cRel (FIG. 2E). Finally, Amplicon 4, located upstream of Region 7, showed no differences in the absence of Wnt5a but a decrease in RPE cells undergoing UOS in the presence of rWnt5a. NPD1 restored the cRel binding to control levels, indicating that in the presence of NPD1, cRel displaces the initially bound p65 (FIGS. 2, E and F). This data indicate that there is a binding interactive competition between p65 and cRel that depends on availability and other factors, such as methylation for functionally relevant consequences of NPD1-mediated cell survival.

Extracellular Availability of Endosome-Free Wnt5a is Controlled by NPD.

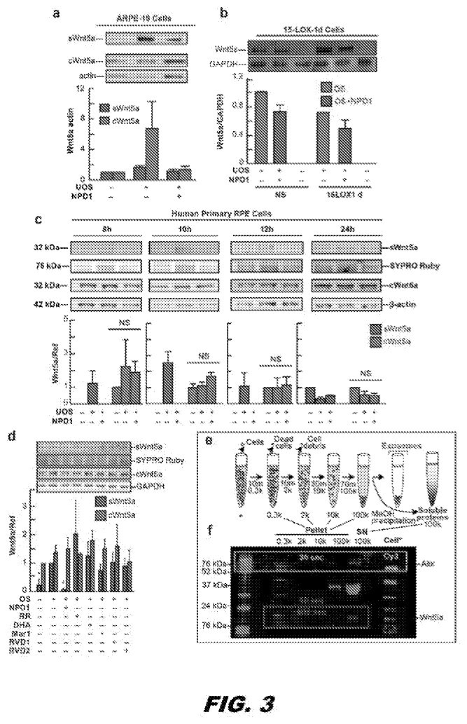

To monitor transcription, translation, and secretion of Wnt5a, we performed western blot assays using cellular lysates and medium from hpRPE undergoing UOS+/−NPD1 at different time points. Medium was precipitated using Methanol/Chloroform to bring down all secreted Wnt5a. In ARPE-19 cells, UOS induced an increase in secreted Wnt5a that disappeared with the addition of NPD1. Wnt5a cellular abundance remained constant for all the treatments in ARPE-19 cells and also in 15-LOX-1d cells, indicating a tight regulation of the balance between secreted (sWnt5a) and intracellular Wnt5a (cWnt5a) (FIGS. 3, A and B). ARPE-19 treated with UOS showed release of Wnt5a (sWnt5a) after 6 hours. hpRPE showed a delay in secretion, at 8 hours and peaked at 12 hours (FIG. 3C). Other docosanoids added to hpRPE showed that only NPD1 decreased Wnt5a in the medium (FIG. 3D) indicate that the transcription, translation and secretion of Wnt5a are under strict regulation by NPD1.

Without wishing to be bound by theory, active Wnt5a was released in exosomes (Gross et al. 2012). To address if Wnt5a was released in exosomes by hpRPE cells we performed medium ultracentrifugation from cells undergoing UOS for 10 hours (FIG. 3E). Western blot of the first pellet after spinning at 300 rpm (dead cells pulled-down), showed a 35 KDa band as that of mature Wnt5a. The bands pattern closely resembles the one in whole cells (FIG. 3F). Pellets of 2000 and 10000 rpm, containing cell debris (FIG. 3E), displayed no bands and the pellet from ultracentrifugation at 100000 rpm (exosomes) lack a 35 KDa band. The supernatant was then precipitated using Methanol/Chloroform and a 35 KDa emerged, indicating that most of Wnt5a is not contained in exosomes. To explore further this issue, extraction of exosomes using a commercial reagent that alters the solubility of vesicles making them precipitate using lower speed centrifugation (10000 rpm) did not yield any band. These results indicate that Wnt5a is not released in exosomes by hpRPE cells undergoing UOS.

Wnt5a/FZD5 Clathrin-Mediated Endocytosis is Required for NF-κB Activation.

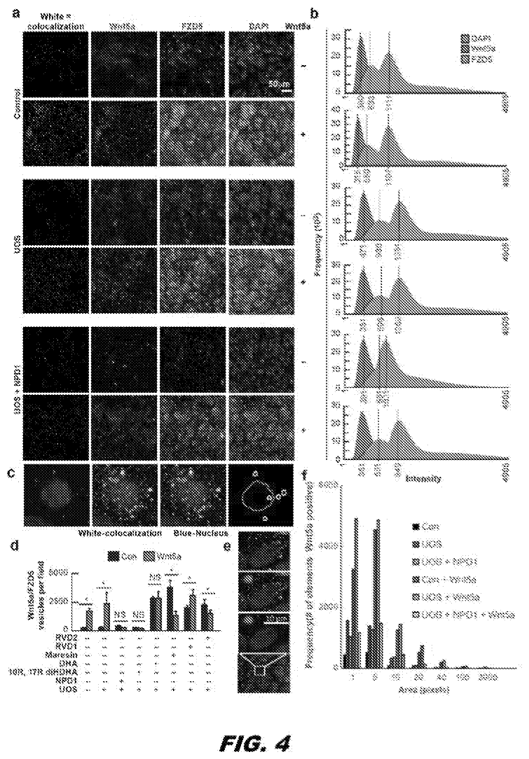

Wnt5a is processed to maturity in the ER/Golgi where it may bind to its receptor and join the recycled protein pool (Willert and Nusse, 2012). To ascertain localization and interaction between FZD5 and Wnt5a we incubated hpRPE undergoing UOS with Wnt5a+/−NPD1 or other lipid mediators. Immunocytochemistry in detergent-permeabilized cells (so the membrane signal for both proteins was lost, assessed by ImageJ to create a frequency histogram for intensity of all pixels in each field) displays a intensity mode of Wnt5a higher in the absence of rWnt5a (FIGS. 4, A, B, C and D). Intriguingly, the mode of FZD5 intensity differed only in the absence of Wnt5a when cells underwent UOS (FIGS. 4, A and B). NPD1 restore both modes to control levels. Z-stacks analysis using BioImage XD software disclosed a stronger colocalization when rWnt5a was added (FIG. 4A and FIGS. 13-15). Co-localization appeared as a punctiform white signal (FIGS. 4, A and C). The number of objects per field, counted using ImageJ (FIG. 4D), increased with the addition of Wnt5a. NPD1 brought down the number of vesicles per field, despite the addition of Wnt5a (FIG. 4D). The NPD1 stereoisomer, of 10-R, 17R configuration, showed the same effect as NPD1. No other lipid mediator decreased the number of vesicles per field in the presence of Wnt5a as it did NPD1 and its stereoisomer, although some of them increased the co-localized signal in the absence of Wnt5a (FIG. 4D). In the absence of Wnt5a, larger, only-wnt5a positive spots were visualized. The quantity of these large spots increased with the addition of NPD1 (FIGS. 4, E and F) evidencing a distinctive pool of Wnt5a that does not interact with FZD5 or was sorted out and separated from the receptor.

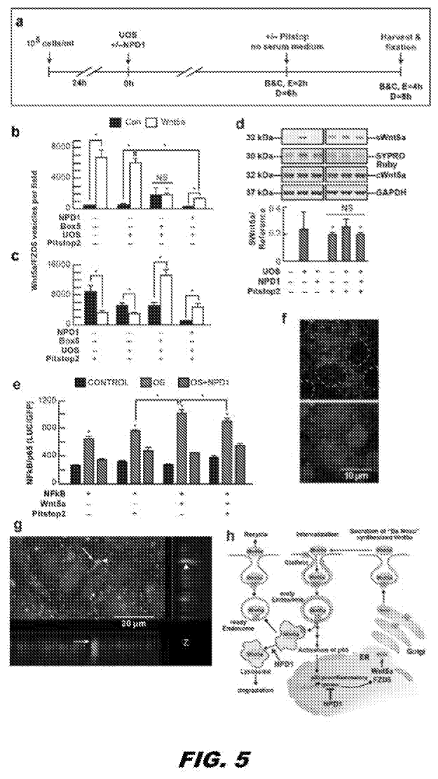

Addition of Wnt5a induces the internalization of several receptors including FZD4 (Chen et al. 2003). We observed an increased signal when rWnt5a was added in control and UOS confronted cells (FIGS. 4, D and F) in the absence of NPD1. To determine whether the increased signal was due to the binding of Wnt5a to FZD5, we treated cells with Box5 under UOS (FIG. 5B). Wnt5a increased the number of vesicles that co-localize with FZD5 in comparison with no addition of the ligand at similar levels in control and UOS treated cells, indicating that there is receptor-ligand complex internalization. Box5 addition decreased Wnt5a induction demonstrating that co-localization is triggered by the interaction of Wnt5a and FZD5. NPD1 had the same effect on Wnt5a-induced increase in co-localized vesicles.

Wnt5a induces internalization of FZD via clathrin-mediated endocytosis (CME) (Feng and Gao, 2015). To determine if Wnt5a/FZD5 internalization involved CME, we treated cells with Pitstop2, an inhibitor of clathrin-mediated processes (Dutta et al. 2012.). Pitstop2 prevented the increased in Wnt5a/FZD5 co-localized vesicles when rWnt5a was added (FIG. 5C) in control and UOS treated cells indicating that Wnt5a/FZD5 complex is internalized via CME. However, the inhibition produced by Box5 was reverted by Pitstop2 (FIG. 5D). In the absence of rWnt5a, the Wnt5a/FZD5 co-localized vesicles increased in all cases as well as in NPD1 treated cells. These results indicate sorting that channels Wnt5a to degradation and recycles FZD5 to send newly synthetized Wnt5a for secretion (FIG. 5H). To determine clathrin involvement in the regulation of sWnt5a, Western blots of the protein from incubation medium from cells undergoing UOS+/−NPD1 and Pitstop2 were studied. Clearly NPD1 decreased media sWnt5a (FIGS. 3, A, C and D). Pitstop2 reverted the effect of NPD1 on sWnt5a (FIG. 5E) but did not modify cWnt5a abundance, indicating that NPD1 does not regulate Wnt5a release, but it enhances endocytosis and degradation of internalized Wnt5a. Surprisingly, controls showed a band when Pitstop2 was applied, indicating that CME is involved in the internalization and degradation of sWnt5a as a mechanism targeted by NPD1 to sustain Wnt5a availability (FIG. 5H).

Wnt5a binding to FZD5 and ROR2 triggers p65/NF-κB activation (FIG. 1J). sWnt5a and FZD5 are internalized via CME (FIG. 5D). Thus, we asked if sWnt5a plus FZD5 internalization is linked to p65/NF-κB activation. We transfected human RPE cells with the NF-κB/p65 reporter construct and treated cells with UOS+/−200 nM NPD1 plus 50 ng/ml rWnt5a. We found that blocking the internalization of Wnt5a via CME with Pitstop2 in cells undergoing UOS in the presence of Wnt5a decreases the activation of p65/NF-κB, demonstrating that the internalized Wnt5a is responsible for NF-κB activation (FIG. 5E). In this context, NPD1 may be enhancing the degradation of the internalized Wnt5a by favoring the conversion of the early endosome to lysosome pathway. Large vesicles that were labeled positive for Wnt5a are shown (FIG. 5G). By increasing Wnt5a degradation, NPD1 may induce a decrease in Wnt5a-triggered NF-κB induction independently of cRel activation. Altogether, the results agree with the mechanism indicated in FIG. 5H.

Ischemic Stroke Activates Wnt5a Expression.

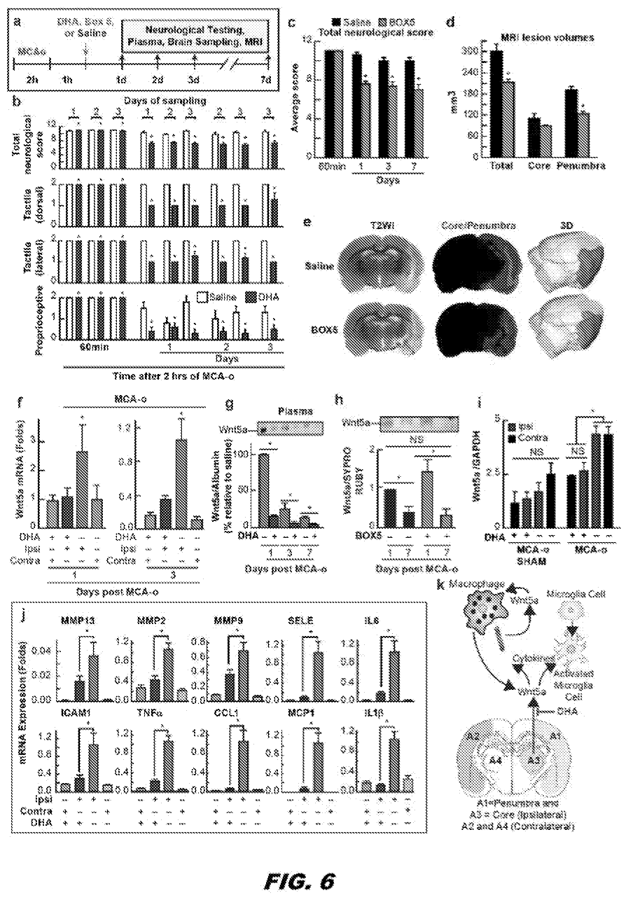

Intravenously (IV) DHA reduces ischemia-reperfusion (I-R) brain damage. To test whether IR increases Wnt5a secretion and signaling, we induced stroke in rats by middle cerebral artery occlusion (MCAo) for two hours and then one hour later injected IV saline (vehicle) or DHA. Neurological scores, tactile and proprioceptive tests were evaluated (FIG. 6B) on rats 1, 2, 3, and 7 days after MCAo (FIG. 6A). Saline-treated showed severe neurological impairments. DHA treatment improved neurologic scores including tactile (dorsal and lateral) and proprioceptive forelimb placing reaction during the one, two or three day's survival period (FIG. 6B). To test whether Wnt5a/FZD5 are involved in neuroinflammatory signaling post ischemia-reperfusion, we injected Box5 to rats subjected to MCAo (FIG. 6A). Overall neurological assessment showed remarkable similarity to that obtained by DHA treatment (FIG. 6D). Moreover, MRI depicted a steep decreased in the infarcted volume (FIGS. 6, D and E) resembling those obtained by injection of DHA (Belayev et al. 2017). UOS in ischemia-reperfusion exerts damage to neurons and astrocytes along with other events (Marcheselli et al. JBC 2003; Belayev et al. 2012). Furthermore, we assessed Wnt5a mRNA in: A1 penumbra, A3 stroke core, and as control A2 and A4 that correspond to contralateral parts of A1 and 3 (FIG. 6K). The penumbra, an area surrounding the ischemic core, is subject to moderate damage and survive the ischemic-reperfusion when treatment is applied. We found that the Wnt5a mRNA was increased in the ipsilateral hemisphere one and three days post-surgery and that DHA blocked this surge (FIG. 6F). After stroke, both hemispheres work synergistically to overcome damage (Buga et al. 2008). In this case, the increase in Wnt5a mRNA level was detected only in the ipsilateral hemisphere; the contralateral showed no surge in Wnt5a expression one and three days after surgery, indicating a local induction of mRNA expression. However, Wnt5a protein abundance showed that the levels of ipsilateral and contralateral hemispheres A1 and A2 did not differ one to the other, and they were high in saline and low in DHA-treated animals (FIG. 6I). In addition, Wnt5a was enhanced in blood after MCAo one day post-stroke and was decreased at day three (FIG. 6G). Impairment of the interaction Wnt5a/FZD5 with Box5 that induced changes in the size of the infarct observed in the MRI (FIGS. 6, D and E) and an improvement in neurological score (FIG. 6C) failed to reduce the plasma Wnt5a content indicating a difference in the action between DHA and Box5. The expression of NF-κB-activated inflammation mediators IL6, TNFα, CCL1, MCP1, and IL1β follows the same trend as Wnt5a after DHA (FIG. 6J), in agreement with the activation of NF-κB/p65. MMP13, MMP2 and MMP9 expression is enhanced when Wnt5a-ROR2 is activated (Yamagata et al. 2012). The three mRNAs showed the same trend of expression as Wnt5a (FIG. 7J) indicating the activation of ROR2 by Wnt5a. Other genes, such as E-selectin and ICAM-1, involved in inflammatory signaling and known to be activated by Wnt5a (Kim et al. 2010), were found to follow the same pattern of Wnt5a expression. These results indicate that the effect of Wnt5a on those genes is restricted specifically to penumbra and not in the contralateral side. As Wnt5a is available in both hemispheres, it seems that stress is required for Wnt5a to act as an inflammatory mediator. These results altogether point at Wnt5a as a non-conventional inflammation mediator and DHA/NPD1 signaling as a regulatory mechanism specifically to switch off Wnt5a-triggered gene expression and Wnt5a extracellular availability.

Discussion

Wnt signaling drives cell survival and differentiation and often modulates (3-catenin activity (Nusse and Clevers, 2017). Recently, other functions attributed to Wnt ligands includes Wnt5a in immune cells, as in macrophages, activate NF-κB/p65 signaling via MAPK with consequent cytokines expression increase (Zhao et al. 2014). Here, we present evidence that Wnt5a triggered NF-κB/p65 activation upon UOS in RPE cells (FIG. 2G) links the dependence of a toxic stimulus to the inflammatory response. Conversely, Wnt5a activated COX-2 expression (FIG. 2C) that displays promoter-binding sites for other transcription factors along with NF-κB/p65 (Diaz-Munoz et al. 2012). These results uncover additional mechanisms of Wnt5a signaling in RPE cells, since the MAPK pathway is activated (Nishita et al. 2010.), COX-2 promoter contains CRE sites (Diaz-Munoz et al. 2012) and MAPK pathway activates CREB (Mercau et al. 2014), leading to COX-2 promoter activation independent of NF-κB/p65.

The addition of rWnt5a enhanced apoptosis beyond the levels induced by UOS resembling TNFα action (FIG. 1B and FIG. 9). However, the sole presence Wnt5a did not trigger RPE cell death (FIG. 1B and FIG. 9). Thus, cells in a susceptible state, Wnt5a might affect their fate since they may succumb to initial insult such as UOS. Insults and exposure to Wnt5a might enhance susceptibility to containment of cell integrity and survival. On the other hand, UOS by itself can increase Wnt5a abundance, and that suffices to induce cell death. The latter was evident when Box5 prevented cell death induced by UOS in the absence of Wnt5a (FIG. 1B).

NPD1 decreased Wnt5a expression in RPE cells (FIGS. 1D and 2C; FIGS. 7 and 8) and we previously found that this lipid mediator synthesis correlated to cRel activity, a member of NF-κB/p65, in RPE cells and in post-stroke penumbra (Calandria et al. 2015). Here, we demonstrated that cRel, binds to at least two regions in the Wnt5a promoter with high affinity (FIG. 2, D-F and Table 4). cRel overexpression suppresses the Wnt5a transcription in response to UOS, and its silencing leads to increased Wnt5a mRNA (FIG. 2A-C). Wnt5a expression is attenuated by NPD1 in cRel-silenced cells indicating that there are other elements controlling Wnt5a transcription and some are NPD1-dependent (Asatryan and Bazan, 2017). The exact nature of other components involved in the suppression of the positive feedback loop is under investigation.

Wnt ligands are secreted via exosomes (Gross et al. 2012); however, Wnt5a is not in the exosomal form in hpRPE cells when we extracted them by ultracentrifugation (FIGS. 3, E, and F) and by differential solubility. Only by precipitation of 100,000 rpm supernatant did we rescue the sWnt5a (FIG. 3F). Therefore, in RPE cells under UOS, Wnt5a is extracellularly released via secretory vesicles (FIG. 5H). Wnt5a signal was found alone and together with FZD5, indicating at least two distinctive events occur in RPE cells (FIGS. 4, A, D, G and H). Wnt5a signal increases with the addition of recombinant Wnt5a (FIGS. 4F and 6B) in agreement with the ability of Wnt5a to enhance not only its own but also FZD4, 2, and 5 receptor endocytosis (Chen et al. 2003; Shojima et al. 2015; Kurayoshi et al. 2007). Vesicular Wnt5a was detected in RPE cells (FIGS. 4 and 5) and secretion of mature Wnt5a is in vesicles, and Golgi supported maturation of the protein (Kurayoshi et al. 2007). Therefore, different size vesicles carrying Wnt5a detected in RPE cells (FIG. 4F) may harness maturation, degradation and sorting of Wnt5a, by a dynamic steady state with intracellular Wnt5a remaining constant (FIG. 3, A-D), while extracellular release is controlled and transcription is variable depending on UOS (FIGS. 1D and 2C; FIGS. 7 and 8) NPD1 decreases soluble Wnt5a during UOS (FIG. 3, A-D and 5D) while Pitstop2 an inhibitor of CME, interrupted Wnt5a endocytosis in resting cells and in NPD1 treated cells, ensuing an increase in secreted Wnt5a under these conditions (FIG. 5D) pointing to a role of NPD1 in the fate of secreted Wnt5a.

Wnt5a, a macrophage activator in the non-sterile innate response (Pereira et al. 2008), also mediates non-sterile inflammatory responses in non-immune cells (Zhao et al. 2014). Our current results indicate that non-immune cells evoke the inflammatory response in sterile conditions. The signaling pathways present similarities: in the stroke penumbra, Wnt5a synthesis is increased (FIGS. 6, F and I) in the ipsilateral side (FIG. 6F), Wnt5a protein was elevated in both sides (FIG. 6I). Thus, the protein was present also in the blood plasma after stroke, providing a candidate stroke marker. Systemic Wnt5a may be involved in the recruiting and stimulation of innate immune cells since Wnt5a induces activation of microglia, dendritic cells and macrophages (Shimizu et al. 2016) enhancing other pathways such as Wnt non-canonical Wnt and TLR-triggered signaling. Wnt5a protein in the bloodstream after 24 hours of stroke onset and DHA treatment brought the levels down (FIG. 6G). This was an early event considering that at three and seven days after stroke the levels of Wnt5a in blood decreased. Intraperitoneal injection with Box5, did not affect the levels of Wnt5a as DHA injection did, indicating that DHA acts differently. Without wishing to be bound by theory, DHA can be converted into NPD1 and enhances the activation of cRel (Calandria et al. 2015), which in turn halts the expression of Wnt5a and thus its release and autocrine and paracrine binding to FZD5, while Box5 interferes with the latter. The data show that Box5 may be beneficious at the behavioral level as well as in the reduction of stroke damage (FIG. 6, C-E); however, DHA produces a sustained improvement. The source of circulating Wnt5a is unknown and currently under investigation. There are at least two sources of secreted Wnt5a: blood cells such as monocytes (Sessa et al. 2016) or endothelial cells that produce the Wnt ligand in certain conditions inducing permeabilization and angiogenesis (Korn et al. 2014; Skaria et al. 2017). The elevation in Wnt5a found in the contralateral hemisphere (FIG. 6F), may be explained by damage or permeabilized brain blood barrier that allowed the entrance of the circulating Wnt ligand. Alternatively, the hike in the contralateral Wnt5a protein, without increasing its transcription, may be explained by glutamate excitotoxicity in the contralateral area at a less degree than in the ipsilesional side (Li et al. 2012). Finally, the expression of genes linked to Wnt5a, UOS and inflammation via NF-κB/p65 or Wnt signaling markedly increase in ipsilateral but not contralateral hemispheres, and such an increase was counteracted by DHA as indicated by the gene expression profile (FIG. 6J).

Transcellular inflammation signaling is not well understood. Without wishing to be bound by theory, non-immune cells under UOS conditions can pass along inflammatory signals that may only affect susceptible cells and lead to their damage. NPD1 interferes with the Wnt5a feedback loop at two strategic signaling points promoting cells survival in bystander cells. The ischemic stroke model provided an ‘in vivo’ test-field to apply our observations at the RPE cell level. In post-stroke, we observed a similar trend seen in the RPE: Wnt5a transcription was elevated at 1 and 3 days after stroke only in penumbra at the ipsilateral hemisphere while the expression in the contralateral side was not affected (FIG. 6F). These results indicate that only susceptible cells affected directly by ischemia reperfusion, promote the Wnt5a positive feedback loop at the transcriptional level. DHA enhances NPD1 synthesis and induces translocation of cRel in neurons (Calandria et al. 2015), which prevent p65 driven activation of Wnt5a transcription. Stressors like NMDA glutamate receptor activation trigger transcription-independent Wnt5a translation (Li et al. 2012), which means that Wnt5a transcription and translation may be uncoupled events and may explain some of our observations (FIGS. 6, F and I). Therefore, we indicate that translation alone is linked to secretion and sequestration from the extracellular space via CME in the RPE (FIG. 5H) and may be plausible in ischemic stroke as well.

These findings uncovered a new participant in the transfer of inflammatory signals occurring in retina and brain under UOS and how endogenous neuroprotection mediators derived from DHA may halt the process to enhance cell survival. The understanding of these new neuroprotective cellular and molecular mechanisms will allow the exploration of new therapeutic avenues to target onset and early progression of brain and retina damage that include neurodegenerative diseases.

Cell Culture, Treatments, and Transfection

Primary human RPE cultures can be from human eyecups, provided by the National Disease Research Interchange (NDRI), as described previously (Calandria et al. 2012). hRPE cells can be grown and maintained in high-glucose MEM (Life Technologies Corporation, Carlsbad, Calif.) supplemented with 10% FBS (Tissue Culture Biologicals, Inc., Long Beach, Calif.), 5% NCS, non-essential amino acids, Penicillin-Streptomycin (100 U/mL), human fibroblast growth factor (FGF) 10 ng/mL and incubated at 37° C. with a constant supply of 5% CO2. ARPE-19 cells, can be plated and grown in DMEM/F-12 containing 10% FBS and 1× penicillin/streptomycin at 37° C., 5% CO2, 99% relative humidity for 24 h. Silenced 15-LOX-1 cells, described in detail elsewhere (Calandria et al. 2009), are derived from ARPE-19 by stably silencing 15-LOX-1. 15-LOX-1 deficient cells can be maintained in the same medium as ARPE-19 with the addition of 500 μg/mL Geneticin (Life Technologies Corporation, Carlsbad, Calif.). Transfection can be performed in all cases using Lipofectamine 2000 (Life Technologies Corporation, Carlsbad, Calif.) following the manufacturer's recommendations. Transfection efficiency can be assessed using an expression plasmid-carrying GFP ORF or by including a negative control siRNA conjugated with Alexa Fluor® 488 or 546 (QIAGEN, Hilden, Germany). siRNA efficiency can be assessed using SYBR green-based real-time PCR using the primers of Table 2 and the percentage of silencing achieved (FIG. 7). Experiments with transfection efficiency that yielded less than 80% can be discarded. For siRNA, we used 6 μl of Lipofectamine 2000 per 50 pmol of siRNA per mL of incubation medium, and, for plasmids, we used 6 μl of Lipofectamine 2000 per 2 μg of plasmid per mL of incubation medium. Cells can be incubated for 5 hours at 37° C., 5% CO2 and 99% relative humidity. After the changing the medium, the cells can be left to recover for 24 hours before starting treatments. When siRNA/plasmid co-transfection is required, siRNA and plasmid-carrying transfection mixes can be prepared separately and added one after the other in the same well. Oxidative stress treatments can be performed using 600 (ARPE-19) or 1600 (hRPE) μM H2O2, along with 10 ng/mL TNFα, for the required time indicated in each experiment. The treatment we used employed the low serum (0.5% serum) without hFGF. Treatment to induce uncompensated oxidative stress (UOS) can be performed after 8 hours of low-serum medium incubation unless specifically noted. Wnt5a, Wnt3a or NPD1 can be added immediately before induction of UOS. In the secretion experiments, Pitstop 2 can be added to the medium 2 hours before harvesting.

Immunocytochemistry and Hoechst Staining

Immunostaining can be performed in 8-well slide chambers and the Hoechst staining in 24-well plates. To carryout immunocytochemistry, a previously described protocol can be followed (Calandria et al. 2012). Briefly, cells can be fixed using 4% paraformaldehyde in PBS 1× for 20 min at room temperature or overnight at 4° C. After three washes with PBS, cells can be permeabilized using 0.1% Triton™ X-100 for five minutes and blocked using 10% normal serum and 1% BSA for one hour. Primary antibody incubation took place overnight at 4° C. and secondary antibody coupled to Alexa Fluor® 488 and 594 (Molecular Probes, Inc, Eugene, Oreg.) can be used to detect Wnt5a and FZD5 respectively. Hoechst staining can be performed using same protocol of fixation. In one embodiment, permeabilization is done using methanol for 20 min at room temperature. Immediately after 20 ng/mL Hoechst 33342 (Life Technologies Corporation, Carlsbad, Calif.) in PBS 1× can be added. Images for Hoechst staining can be obtained using Nikon Ti-U inverted fluorescence microscopes with NIS-Elements BR 3.00 software (NIKON Inc, Melville, N.Y.) and ICC images from Olympus FV1200 confocal with Fluoview software FV10-ASW Version 04.02.02.09 (Olympus Corp Center Valley, Pa.). The analysis can be performed using ImageJ 1.48 (National Institutes of Health, Bethesda, Md.). Dying cells can be identified by size (0 to 50 pixels), intensity threshold, and circularity. The ratio of hyperpyknotic cells over the total can be calculated for nine randomly chosen fields per sample obtained from three independent wells per experiment. Each experiment can be repeated at least three times to confirm the findings.

In an embodiment involving early stages of apoptosis, the total number of cells per field is assessed and counted to ensure unbiased observations. In embodiments, colocalization of Wnt5a and FZD5 is performed using BioImageXD (Kankaanpää et al. 2012; Universities of Jyväskylä and Turku in Finland and the Max Planck Institute CBG in Dresden, Germany) on z-stack images obtained at 20× magnification in an Olympus FluoView1200 (Olympus Corporation, Tokyo, Japan) confocal laser-scanning microscope. Settings can be adjusted in the conditions that show higher intensity and can be used throughout the remaining samples. In an embodiment, Pearson's colocalization coefficient (PCC) is obtained using FV1200 analysis software for every field in each experiment, and the mean and SEM are depicted in FIGS. 7-11. Validation of the FZD5 primary antibody is depicted in FIG. 8. In certain embodiments, colocalized objects and plots of pixels vs intensity are obtained using ImageJ (Schneider et al. 2012).

Protein Precipitation and Western Blot

In order to assess secretion of Wnt5a, 1 mL of medium can be collected, centrifuged at 13,000 rpm/5 min at 4° C. to remove cell debris and precipitated using methanol/chloroform (Friedman DB. 2007). The pellet can be re-suspended and denatured in 100 μl of 2× Laemmli sample buffer (Bio-Rad Laboratories, Hercules, Calif.) at 95° C. for 5 min. MCAo brain tissue and cell samples can be homogenized using RIPA (Thermo Fisher Scientific, Waltham, Mass.) buffer supplemented with protease and phosphatase inhibitor cocktails (Millipore Sigma, Burlington, Mass.). In embodiments, the amount of 30 μg of total protein is loaded in NuPAGE® Novex® 4-12% Bis-Tris precast gels (Life Technologies Corporation, Carlsbad, Calif.) and run at constant 120V for about 1 hour and 20 min. Proteins can be transferred to μm Nitrocellulose membrane using Bio-Rad Trans-Blot® Turbo™ System (Bio-Rad Laboratories, Hercules, Calif.). ECL™ Plex Fluorescent Rainbow Markers (GE Healthcare, Chicago, Ill.) can be used as the ladder for protein's molecular weight. Membranes can be blocked using 5% non-fat dry milk (Bio-Rad Laboratories, Hercules, Calif.) in TBS with 1% Tween® 20 (TBST-10×) (Croda International, Snaith, United Kingdom) for 1 hour and incubated overnight with primary antibodies. Anti-mouse or anti-rabbit secondary antibodies conjugated with Cy3 or Cy5 can be used to visualize the protein of interest. Immunoblots can be documented using LAS 4000 imaging system (GE Healthcare Life Sciences, Marlborough, Mass.). A time course exposition can be produced in each case to prevent quantification of saturated images. In embodiments, densitometry data are obtained using ImageQuant™ TL software (GE Healthcare Life Sciences, Marlborough, Mass.).

SYBR Green-Based Real-Time PCR

In various exemplary embodiments, brain samples are homogenized on ice by a Dounce-type homogenizer and total RNA is extracted by TRIzol Reagent (Life Technologies Corporation, Carlsbad, Calif.). Cell samples, total RNA can be extracted by RNeasy Mini Kit (QIAGEN, Hilden, Germany) following manufacturer's protocol. The purity and concentration of RNA can be determined by NanoDrop ND-1000 Spectrophotometer (Thermo Fisher Scientific, Waltham, Mass.). cDNA first strand can be obtained from one microgram of total RNA using iScript™ Reverse Transcription Supermix (Bio-Rad Laboratories, Hercules, Calif.). The resulting cDNA can be used as template SYBR-green or Eva-green based real-time PCR quantification using SsoAdvance Universal Supermix (Bio-Rad Laboratories, Hercules, Calif.). In embodiments, data are collected and analyzed using CFX Manager 3.0 software, the ΔΔCt method. In one embodiment, a melting curve is produced for every run to assure a unique amplified product per primer set. Primers are depicted in Table 4.

Luciferase Assay

For activation of canonical NF-κB, cells can be co-transfected with plasmid p65/p50 promoter consensus sequence Cignal NF-κB Reporter Kit, ((QIAGEN, Hilden, Germany) along with both positive (constitutively active promoter) or negative controls and GFP by using Lipofectamine 2000 (Life Technologies Corporation, Carlsbad, Calif.) following company protocols. COX-2 promoter activity can be measured using a construct carrying 830-bp fragment of cyclooxygenase-2 (COX-2) promoter driving the luciferase expression described elsewhere (Calandria et al. 2012). β-catenin activity can be measured using TOP Flash/FOP Flash constructs obtained from Addgene (Cambridge, Mass.) (Veeman et al. 2003). Except TOP/FOP flash, the Luciferase activity can be standardized using a construct expressing GFP constitutively under CMV virus promoter. To assess luciferase activity, cell lysates can be obtained using Passive Lysis Buffer (Promega, Madison, Wis.) and mixed with a luciferase assay reagent (Promegam Madison, Wis.). Chemiluminescence produced by luciferase and fluorescence from GFP can be detected using Appliscan 2.3. Data can be analyzed using SkanIt 2.3 (Thermo Fisher Scientific, Waltham, Mass.).

DNA Binding Motifs and Methylation

To analyze the consensus binding sequences of the Wnt5a promoter A (Katula, et al. 2012), two searching engines can be used: TRED (http://rulai.cshl.edu/TRED), which uses the JASPAR database, and TFBind (http://tfbind.hgc.jp/) that uses TRASFAC database (Jiang et al. 2007 and Tsunoda et al. 1999). Tables 6 and 7 contain scores and positions obtained for the 7 regions identified that cRel potentially binds (FIG. 2). In certain embodiments, analysis of the methylation is performed using MethPrimer (http://www.urogene.org/methprimer2/; PUMCH, Chinese Academy of Medical Sciences, Bejing, China) (Li and Dahiya, 2002).

ChIP Assay

The chromatin immunoprecipitation assay can be performed using SimpleChIP® Plus Enzymatic Chromatin IP Kit (Cell Signaling Technology, Boston, Mass.) following the manufacturer's recommendations including ChIP validated cRel antibody. Positive control Histone H3 antibody and normal serum can be provided by a kit, as well as control primers for human RPL30 Exon 3. Primers A1 to 4 can be designed using Primer-Blast (NCBI, Bethesda, Md.)((Ye et al. 2012) and are depicted in Table 5. The immunoprecipitated samples real-time PCR values can be standardized using 10% of the input chromatin preparation using primers A1, A2, and A3 pooled values and RPL30 Exon 3 values.

Animal Preparation

In certain embodiments, Male Sprague-Dawley rats (290-320 g; Charles River Lab, Wilmington, Mass., USA) are used for in vivo studies. For all surgical procedures, animals can be fasted and anesthesia can be induced with 3.5% isoflurane and 70% nitrous oxide and 30% oxygen. Animals can be orotracheally intubated; given atropine for secretions, pancuronium for immobilization; ventilated mechanically on a humidified mixture of 70% nitrous oxide, 1.0-1.5% isoflurane and a balance of oxygen. A femoral artery and vein can be catheterized for continuous blood pressure monitoring and periodic blood sampling for arterial gases and pH. PCO2 can be maintained at 35-40 mm Hg and P02 at 105-120 mm Hg by ventilator adjustments. Rectal temperature can be measured with a thermistor and maintained with a heating lamp at 37.0-37.5° C. Cranial (temporalis muscle) temperature also can be monitored and regulated with a separate warming lamp at 36.2-36.7° C.

Transient Middle Cerebral Artery Occlusion (MCAo)

The right MCA can be occluded for 2 h by intraluminal filament, as described previously (Belayev et al. 2011). Briefly, the right common carotid artery (CCA) and external carotid artery (ECA) can be exposed through midline neck incision, and then completely isolated from the surrounding nerves. The occipital branches of the ECA and pterygopalatine artery can be ligated. A 4-cm of 3-0 nylon filament, coated with poly-L-lysine can be advanced to the origin of MCA through the proximal ECA via internal carotid artery. The filament can be inserted 20 to 22 mm from the bifurcation of the CCA, according to the animal's body weight. The neck incision can then be closed, and the rats can be returned to their cages. After about 2 h of MCAo, the rats can be re-anesthetized with the same anesthetic combination and the intraluminal filament can be gently removed. The animals can be allowed to survive for different times, according to the experimental protocol, with free access to water and food.

Behavioral Tests

In certain embodiments, behavioral tests are conducted before, during MCAo (at 60 min), and then at 24 h, 48 h, 72 h, or 7 days after MCAo by an investigator blinded to the experimental groups. The battery can comprise two tests, (1) postural reflex to examine the upper body posture when the rat is suspended by tail, and (2) forelimb placing test to assess the forelimb placing responses to visual, tactile and proprioceptive stimuli (Belayev et al. 2011). Neurologic function can be graded on a scale of 0 to 12 (normal=0, maximal deficits=12), as described previously (Belayev et al. 2011). In embodiments, the severity of stroke injury is assessed by behavioral examination of each rat at 60 min after onset of MCAo. Rats that do not demonstrate high-grade contralateral deficit (score, 10-11) can be excluded from further study.

Treatment Groups

In various exemplary embodiments, docosahexaenoic acid (DHA; 5 mg/kg, Cayman, Ann Arbor, Mich., USA), Box5 (1 mg/kg, Millipore-Sigma, Burlington, Mass.) or vehicle (0.9% saline) is administered intravenously into the femoral vein at a constant rate over 3 min using an infusion pump at 3 h after onset of MCAo. In embodiments, for western-blot study, rats are sacrificed on days 1, 3, or 7; for real-time PCR study, rats are sacrificed on days 1, 2, or 3.

Magnetic Resonance Imaging Acquisition and Analysis of Volumes.

In embodiments, high-resolution ex vivo magnetic resonance imaging (MRI) is performed on 4% paraformaldehyde-fixed brains at each time point (days 1, 3 and 7) using an 11.7T Bruker Advance 8.9 cm horizontal bore instrument equipped with an 89 mm (ID) receiver coil (Bruker Biospin, Billerica, Mass., USA). T2-weighted images (T2WI), diffusion weighted images (DWI), 3D volumes and apparent diffusion coefficient (ADC) maps can be collected and analyzed as previously described and known in the art (Obenaus et al. 2011). Briefly, T2 and ADC maps can be computed from T2WI and DWI, respectively. In certain embodiments, hierarchical region splitting (HRS) are used to automatically identify core and penumbra volumes (total lesion=core+penumbra) from T2 relaxation and water mobility (ADC), as published previously. Penumbral tissue determination by HRS can be confirmed by use of PWI/DWI subtractions at each brain level. The penumbra can be defined as the difference between the PWI and abnormal ADC (diffusion-perfusion mismatch) (2 STD elevation or reduction compared to normal tissues).

Statistics

In the exemplary embodiments disclosed herein, data are presented as mean values±SD. Repeated measures analysis of variance (ANOVA) followed by Bonferroni procedures to correct for multiple comparisons are used for intergroup comparisons. Two-tailed Student's t-tests are used for two-group comparisons. Differences at P<0.05 are considered statistically significant.

### FIGURE LEGENDS

FIG. 1: Docosanoids counteract UOS-dependent NF-κB activation and apoptosis via Wnt5a/FZD5/ROR2.

A, Experimental design and morphology criteria to determine apoptotic cells. B, Wnt5a enhanced cell death triggered by H2O2. Apoptotic cell percentage was measured using Hoechst staining and quantified by ImageJ. C-E Docosanoids prevented an increase in Wnt5a transcription in cells undergoing UOS. C, DHA, and its derivatives NPD1, 10R, 17R diHDHA, Maresin-1, RvD1 and RvD2. D-F, SYBR green real time PCR was used to determine semi quantitatively the expression of Wnt5a D; FZD5 E in human primary RPE cells and Receptors linked to Wnt signaling; F, in ARPE-19 cells induce a decrease of UOS-triggered Wnt5a transcription. Standardization was performed using p-actin and GAPDH as housekeeping genes. G, Schematic representation of luciferase assay performed with TOPFlash/FOPFlash, and NF-κB/p65 reporter constructs. H, Wnt5a does not affect activation of β-catenin. TOP-Flash (wild type) and FOP-flash (mutated) β-catenin binding sites activity H, and NF-κB binding activity I and J, were measured by the means of luciferase reporter system assay and standardization was made using a plasmid expressing GFP. I, NPD1 does not prevent activation of NF-κB triggered by UOS but it affects its activation when Wnt5a is added. J, ROR2 and FZD5 is involved in the activation of NF-κB by Wnt5a. Human primary hRPE cells were transfected with siRNA targeting FZD5 and ROR2 separately and together or control non-specific siRNA. UOS was induced in the presence or absence of 100 nM NPD1 and Wnt5a. The bars represent the mean of three measurements and the standard error of the mean. *p<0.05.

FIG. 2: NPD1-dependent cRel binding to promoter A decreases Wnt5a expression.

A, Representation of siRNA resistant cREL ORF strategy to control off target effects of siRNA. cREL ORF mutations were designed to interfere with siRNA binding while preserving the protein sequence by introducing silent mutations. Mutations were performed by exchanging the third base of some codons without altering the amino acid that they code for B, cRel quantification (top) and Wnt5a (lower) mRNA by means of SYBR green-based real-time PCR in hRPE cells undergoing UOS, +/−NPD1. Cells were transfected with a mixture of 3 siRNAs targeting cRel (left), cREL wild type ORF (middle) and mutated siRNA resistant cREL ORF plus the tried of siRNAs altogether (right). C, Wnt5a mRNA quantification of non-transfected cells (Control for B). D, Model of regulation of NF-κB sites by cRel: in silica analysis of Wnt5a promoter (Katula et al., 2012) showing that the two binding sites for NF-κB have high affinity for p65, p50 and cRel. The cartoon shows the possible direction in which transcription factors elicit their action. NF-κB site prediction is in Table 4. D and E, Other NF-κB binding sites detected by TRED. Region 2 corresponds to the upstream NF-κB binding site and Region 6 to the downstream binding site depicted in D. Regions 1, 3, 4, 5, and 7 showed up in the general TRED search with high score (Table 4) for the three NF-κB. Four amplicons were designed close or sitting on these regions to assess each site. In purple CpG islands that encompass the putative binding sites were depicted (Table 5). E, SYBR green-based real-time PCR using as template the proteinase digested genomic DNA fragments resulting from micrococcal DNAs digestion and cRel pull down. UOS=1600 μM H₂O₂ plus 10 ng/ml TNFα. NPD1: 100 nM and Wnt5a: 50 ng/ml unless stated otherwise. Bars represent mean of three measurements and standard error of the mean. *p<0.05.

FIG. 3: Secreted Wnt5a is reduced by NPD1 in human RPE cells undergoing UOS.

Wnt5a protein is released from ARPE-19 (A,B) and hRPE cells (C) in UOS. A, ARPE-19 cells were treated with 600 μM H₂O₂ and 10 ng/ml TNFα for 6 hours in the presence or absence of 100 nM NPD1. Wnt5a was measured in cellular lysate (cWnt5a) and in medium (sWnt5a) by the means of Western blot. B, Cellular content of Wnt5a in 15-LOX-1d and control cells. C, Time course release of sWnt5a in human RPE cells. D, Selective effect of NPD1 on sWnt5a. E and F, Content of sWnt5a in medium of human RPE cells in the presence of 1600 μM H₂O₂ and 10 ng/ml TNFα. E, Exosome enrichment protocol using ultracentrifugation. F, Content of sWnt5a in the different fractions of the medium. The bars represent the mean of three measurements and the standard error of the mean. *p<0.05.

FIG. 4: NPD1 enhances Wnt5a internalization.

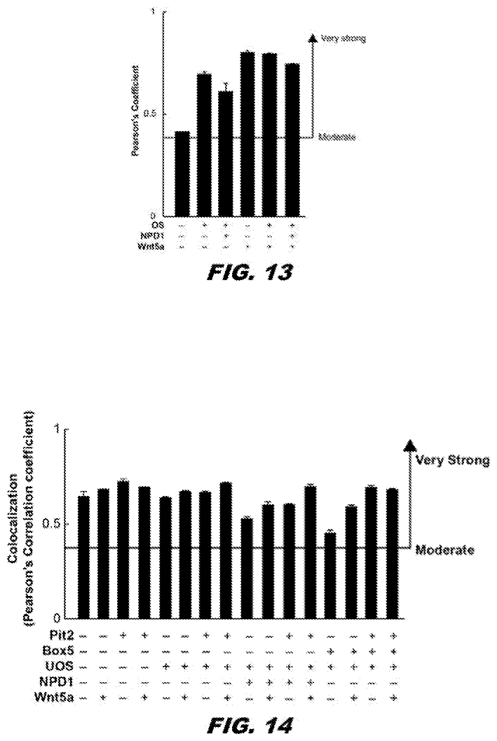

A, Representative images of colocalized signal of FZD5 and Wnt5a in hRPE cells. RPE cells were incubated for 2 hours with 1600 μM H₂O₂+/−100 nM NPD1 and 50 ng/ml Wnt5a. Immunostaining of Wnt5a (Red) and FZD5 (Green) and z-stack analysis of images using BioImageXD. Pictures taken at 20×. Lasers were set up for each experiment using Control+Wnt5a and used without modification to take the remaining pictures. Colocalization of the two signals are shown in left column (white). B, Total signal intensity of three channels (DAPI=blue, Alexa 488=green and Alexa 594=red) in three random fields per well/condition. On the right of each row, the histogram of intensity vs frequency depicts the number pixels showing the intensity value on X-axis. The upper limit intensity is set at 4095. The black vertical lines for each channel indicates the mode (most frequent observation) to designate the intensity at which each curve reaches its maximum. The signal points or clusters of pixels showing colocalization (A-left column) were quantified using ImageJ of three to six random fields encompassing one or two wells, in up to 3 independent experiments. C, Blow up of a single cell showing vesicles positives to Wnt5a (red), FZD5 (green) or both yellow. The fourth panel shows a drawing of the nucleus (blue) and the position of the vesicles showing colocalization of Fzd5 and Wnt5a. D, Quantification of colocalized spots in human RPE cells undergoing UOS+/−eicosanoids in FIG. 1C. Pearson colocalization coefficient was plotted in FIGS. 13-15. E, Blow up of a cell showing large cluster of Wnt5a signal present most frequently in certain treatments. F, Frequency vs Area histogram for representative fields showing different sizes of clusters of Wnt5a positive signal. Bars represent mean of three measurements and standard error of the mean.*p<0.05.

FIG. 5: Pitstop2 halts the internalization of Wnt5a and its subsequent activation of NF-κB.

A, Experimental design. B and C, quantification of clusters showing colocalization in the absence B, or presence c, of 25 μM Pitstop2. 100 μg/ml Box5, an inhibitor of Wnt5a biding to FZD5 was used to interrupt Wnt5a effect. ImageJ Quantification of the objects resulting from the 3D colocalization analysis. Pearson's colocalization coefficient for these experiments was plotted in Figure X+1. D, Western blot analysis of Wnt5a in response to UOS in the presence or absence of NPD1 and Pitstop2. E, Pitstop2 interferes with the activation of NF-κB/p65. Reporter assay of three NF-κB/p65 binding sites in tandem driving the expression of luciferase ORF, the construct was depicted in FIG. 2A. NPD1=100 nM and UOS=1600 μM H₂O₂. F, Perinuclear distribution of Wnt5a G, vesicle like signal in the Z axe of the Z-stack. Whole arrow shows a fusion between a FZD5 and Wnt5a positive to a large Wnt5a positive cluster. Arrowhead shows already fused colocalized cluster. H, Model of internalization and recycle of Wnt5a and FZD5 to activate NF-κB/p65. The bars represent the mean of three measurements and the standard error of the mean. *p<0.05.

FIG. 6: DHA prevents Wnt5a overexpression and secretion in response of ischemia reperfusion.

A, Timeline of MCAo. DHA, Box5 or saline were administered at 1 h after 2 h of MCAo and rats were sacrificed on days 1, 2, 3 or 7. B, Effect of DHA on neurological recovery. Total score (normal score=0, maximal deficit=12), tactile placing (dorsal, lateral, proprioceptive reactions; normal score=0, maximal deficit=2) after MCAo. DHA or saline was administered at 1 h after 2 h of MCAo and rats sampled on days 1, 2, 3 or 7. Values are mean±SD; n=4 rats/group. *Significantly different from corresponding saline group (p<0.05, repeated measures ANOVA followed by Bonferroni tests). C-E, 400 μg Box5 IV administration, 1 h after MCAo showed effects on neurological recovery and infarct size resembling DHA treatment. C, Total neurological score at days 1, 3 and 7; D, MRI quantification at day 7 of the lesion volume depicting total, core and penumbra and; E, representative coronal sections showing T2 weighted image (T2WI), the defined core and penumbra region (red and blue, respectively) in the second column; and a 3D reconstruction of the lesion. F, Wnt5a mRNA assessment by means of SYBR-green real-time PCR in rat cortex Ipsilateral (Ipsi) or contralateral (Contra) of MCAo, treated with saline (vehicle) or DHA. G,H Wnt5a protein in plasma 2 h after MCAo with DHA (N=4); G, or Box5 (N=4); H, at 1, 3 and 7 days post-surgery. I, Western blot of tissue A1 and A2 (Ipsi and Contra) in Saline and DHA treated animals 1 day after MCAo or Sham-MCAo. J, Quantification of Wnt5a and NF-κB linked gene expression in MCAo. MCAo and saline treatments (vehicle) and DHA for 3 days (N=3) and each reaction was run in triplicate. The mRNA were measured using SYBR green RT-PCR. The cartoon show the color-coded region to test gene expression. Bars represent t mean of three measurements and standard error of the mean. *p<0.05. K, Wnt5a inflammatory signaling after increased abundance due to ischemia/reperfusion.

FIG. 7. Wnt5a was up-regulated in ARPE-19 cells deficient in 15-LOX-1 undergoing UOS and downregulated by NPD1.

Design for experiments leading to the identification of Wnt5a (left). UOS-triggered increase in Wnt5a expression was reversed by NPD1 in 15-LOX-1d. 15-LOX-1d cells, which shows depletion in NPD1 synthesis (Calandria et al., 2009), were used to determine genes regulated by the lipid messenger in a microarray assay. UOS=600 μM H₂O₂ plus 10 ng/ml TNFα Representative values of three independent experiments. ANOVA and test for false positives was applied to select regulated genes on microarray output.

FIG. 8. Quantification of mRNA of Wnt5a in primary human RPE cells.

Confirmation of the NPD1-regulation of Wnt5a transcription. UOS was carry out using 1600 μM H₂O₂ for hRPE cells, plus 10 ng/ml TNFα to confirm microarray output using SYBR green-based real-time PCR in human primary cells. Representative values of three independent experiments. Bars represent the mean+standard error of the mean of 3 different experimental subjects.

FIG. 9. Wnt5a enhances the percentage of cell death induced by UOS in ARPE-19 cells.

Hoechst-positive ARPE-19 cells were beyond H2O2-induced levels. The criteria used to determine Hoechst positive cells and the experimental design is depicted in FIG. 1A. The bars represent the mean of three measurements and the standard error of the mean. *p<0.05.

FIG. 10. Quantification of mRNA of FZD5 and ROR2 in silenced cells.

A, Design of luciferase reporter assay performed with COX-2 promoter construct. UOS was induced by the addition of 10 ng/ml TNFα and 600 μM H₂O₂ in ARPE-19 cells. The concentrations of Wnt5a used was 50 ng/ml. Cells were co-transfected with the constructs driving the expression of luciferase and a plasmid that constitutively expressed green fluorescent protein for standardization. Luciferase activity is denoted in luciferase activity units (LUC) and standardized using GFP fluorescent (GFP). B, Wnt5a and IL-1β effect time course on COX-2 promoter activity. COX-2 promoter fragment from −830 bp to the site of transcription start contains one NF-κB binding site at −448 bp. Luciferase activity (LUC) was measured in ARPE-19 cells in the presence of 50 ng/ml Wnt5a or 20 ng/ml IL1-β. C, NPD1 prevents Wnt5a-induced activation of COX-2 promoter in hRPE cells. Luciferase activity measured in cells incubated with 50 ng/ml Wnt5a in the presence or absence of 100 nM (+) and 200 nM (++) NPD1. The bars represent the mean of three measurements and the standard error of the mean. *p<0.05.

FIG. 11. Quantification of mRNA of FZD5 and ROR2 in silenced cells (Related to FIG. 1J).

A, FZD5 and B, ROR2 mRNA quantification on Negative control, FZD5 and FZD5 plus ROR2 siRNA transfected human RPE cells. Controls corresponding to experiment. The bars represent the mean of three measurements and the standard error of the mean. *p<0.05.

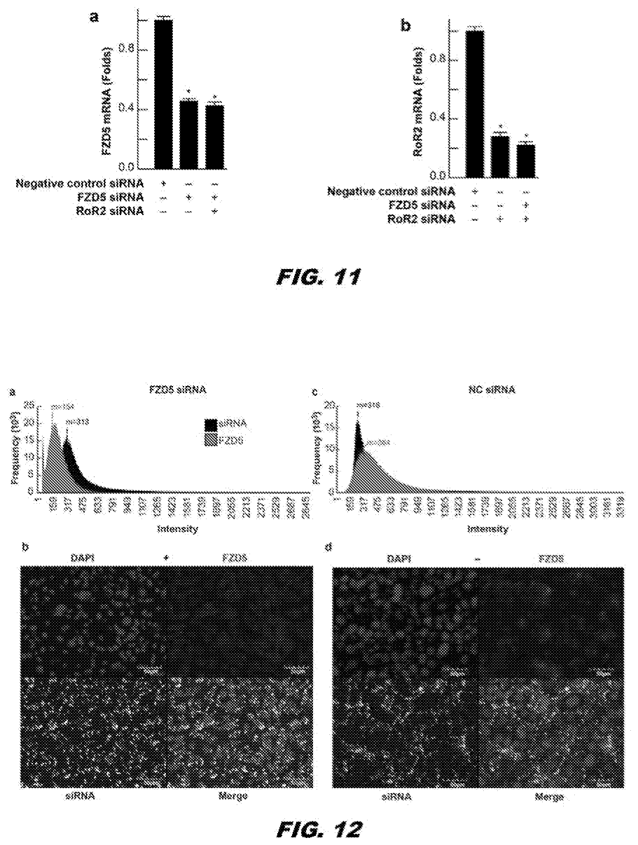

FIG. 12. Validation of FZD5 antibody for Immunocytochemistry (Related to FIG. 4, A-F and FIGS. 5, B, C, F and G).

Histograms of Intensity vs frequency for FZD5 signal (red) and siRNA tracer signal (black). (B and D) representative pictures of human RPE cells transfected with B, FZD5 siRNA and D, Negative control siRNA. To allow the comparison, confocal lasers were set to the negative control parameters and pictures were taken without changing them. White=tracer siRNA; blue=DAPI and red=FZD5.

FIG. 13. Pearson's colocalization coefficient (PCC) for first experiment in the series of colocalization by immunocytochemistry, Related to FIGS. 4 and 5.

The PCC values obtained for all the slices of z-stacks of three fields were averaged and plotted. The bars represent the mean of three measurements and the standard error of the mean. *p<0.05.

FIG. 14. Pearson's colocalization coefficient (PCC) for experiment in FIG. 4C showing colocalization of by immunocytochemistry (Related to FIGS. 4 and 5).

The PCC values obtained for all the slices of z-stacks of three to six fields were averaged and plotted. The bars represent the mean of three measurements and the standard error of the mean. *p<0.05.

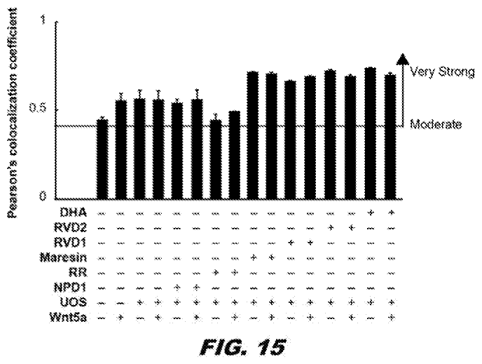

FIG. 15. Pearson's colocalization coefficient (PCC) for experiment in FIGS. 5, B and C showing colocalization of by immunocytochemistry (Related to FIGS. 4 and 5).

The PCC values obtained for all the slices of z-stacks of three fields were averaged and plotted. The bars represent the mean of three measurements and the standard error of the mean. *p<0.05.

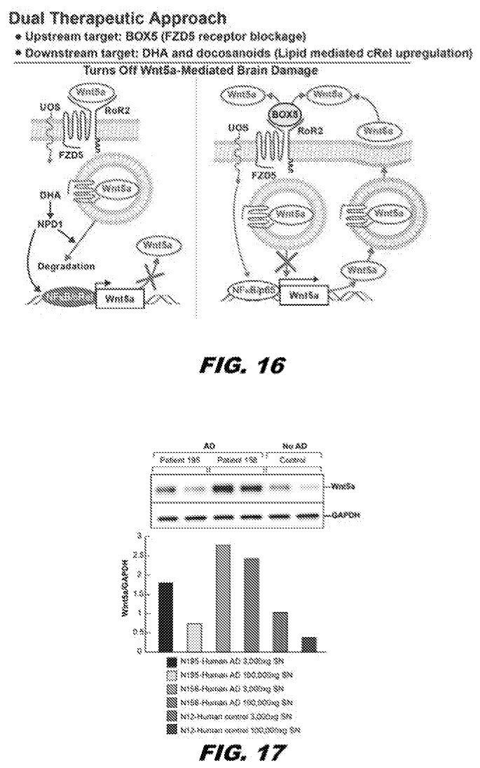

FIG. 16. Mechanisms by which DHA and BOX5 alleviate Wnt5a-mediated cell damage.

Mechanism of action of combinatorial therapy the Box5 peptide and Docosanoids at strategic upstream and downstream different points of the signaling pathway that contribute to an enhancement of the blockage of the inflammatory/cell damaging signaling triggered by Wnt5a. This results in neuroprotection.

FIG. 17. Western blot expression of vesicular and secreted Wnt5a in the post mortem brains of two Alzheimer Disease (AD) patients as compared to a human control.

Wnt5a intracellular life is spent is a vesicular form. The supernatant fraction obtained by centrifuging a tissue lysate at 3000 rpm contains cytoplasm, membranes and mitochondria. In this fraction, vesicles are present. In the 100000 rpm, only soluble cytosolic molecules and small structures are present. Western blot performed from two postmortem cortical Alzheimer Disease patient samples (cases #195 and 158) showed Wnt5a was elevated in both fractions while in a normal control the soluble Wnt5a is almost negligible and is very low abundant in vesicular form. These results indicate a relationship between an increase in Wnt5a synthesis (intracellular vesicular form 3000 g) and secreted (soluble 100000 g) pro-inflammatory Wnt5a and Alzheimer's disease. Thus, without wishing to be bound by theory, patients of AD can present increased release of Wnt5a and thus can display the ligand in blood or cerebrospinal fluid.

### TABLES

TABLE 1
[DNA constructs and siRNAs, Related to constructs and siRNAs],
Related to FIGURES 2A-D, 2G, 211, 3A, 3B, and 6E.

| | | | Reference/catalog |
|---|---|---|---|
| | | | number and |
| Constructs | Type | Gene/protein | company |
| COX2 | 830 bp upstream | PTGS2 | Calandria |
| promoter | transcription | (NM_000963.1, | et al., 2012. |
| reporter | initiation site | NP_000954) | |
| vector | | | |
| Wild Type | REL (untagged)- | REL (NM_002908) | Origene |
| cREL | UNIQUE VARIANT | | True ORF |
| expression | 1 of Human v-rel | | Cat #SC126639 |
| vector | reticuloendotheliosis | | |
| | viral oncogene | | |
| | homolog (avian) (REL) | | |
| siRNA | Human REL ORF | REL (NM_002908) | GeneART gene |
| resistant | designed silent mutant | Human cDNA Clone | synthesis of JMC |
| cREL | affecting binding of | | designed cREL |
| expression | Trilencer 3 siRNAs | | mutant. Calandria |
| vector | | | et al., 2015 |
| NFkB | 3 tandem copies of p65 | | Qiagen, Cignal |
| reporter | binding sequence | | NFκB Reporter |
| vector | driving the expression | | (luc) Kit: |
| | of luciferase. | | Cat #CCS-013L |
| TOP | Super8XTOPflash | 7 TCF/LEF | Addgene repository |
| Flash | construct M50, Beta- | binding sites: | Plasmid #12456. |
| | catenin reporter. | AGATCAAAGGgggta | (Veeman et al, 2003) |
| | TCF/LEF sites | (SEQ ID NO: 2), with | |
| | upstream of a | TCF/LEF binding site | |
| | luciferase reporter. | in CAP letters, and a | |
| | | spacer in lower case, | |
| | | separating each copy | |
| | | of the TCF/LEF site. | |
| FOP | M51 Super | 6 mutated TCF/LEF | Addgene repository |
| flash | 8 × FOPFlash | binding sites that were | Plasmid #12457. |
| | (TOPFlash mutant) | cloned into the pGL3 | (Veeman et al, 2003) |
| | | vector (Promega), | |
| cREL | REL (Human) 3 unique | Human cREL | Origene Trilencer |
| siRNA | 27mer siRNA duplexes | (NM_002908) | Cat #SR304027 |
| FZD5 | Human FZD5 21-mer | Human FZD5 | Silencer select |
| siRNA | siRNA duplexes | (NM_003468) | Validated Ambion, |
| | | | Life Technologies- |
| | | | Thermo |
| | | | Cat #4390824. |
| | | | ID: s15416 |
| ROR2 | Human ROR2 21-mer | Human ROR2 | Silencer select |
| siRNA | siRNA duplexes | (NM_004560) | Ambion, Life |
| | | | Technologies- |
| | | | Thermo |
| | | | Cat #4390824. |
| | | | ID: s9758 |
| Negative | Non-specific binding | | Allstars. Qiagen |
| control | siRNA sequence | | Cat #1027292 |
| siRNA | | | |
| Alexa Fluor | | | |
| 488 | | | |
| conjugated | | | |
| Negative | Non-specific binding | | Allstars. Qiagen |
| control | siRNA sequence | | Cat #1027287 |
| siRNA | | | |
| Alexa | | | |
| Fluor 488 | | | |
| conjugated | | | |

TABLE 2
[Primers information], Related to FIGS. 1B, 1D, 2E, 2F, 3B, 3C, 7D, and 7G.

| Target | Sequence | SEQ ID NO: | Source |
|---|---|---|---|
| Rat Wnt5a | Forward primer | 3 | RealTimePrimers.com |
| | 5′-TTACCCAAACCGGACTGTTA-3′ | | |
| | Reverse primer | 4 | |
| | 5′-AGCCTTTTCGGTTCATCTCT-3′ | | |
| Human Wnt5a | Forward primer | 5 | Campioni et al., 2008. |
| | 5′-CAAAGCAACTCCTGGGCTTA-3′ | | |
| | Reverse primer | 6 | |
| | 5′-CCTGCTCCTGACCGTCC-3′ | | |
| Rat Cxcl1 | Forward primer | 7 | RealTimePrimers.com |
| | 5′-GCGGAGAGATGAGAGTCTGG-3′ | | |
| | Reverse primer | 8 | |
| | 5′-TCCAAGGGAAGCTTCAACAC-3′ | | |
| Rat ACTB | Forward primer | 9 | RealTimePrimers.com |
| | 5′-CACACTGTGCCCATCTATGA-3′ | | |
| | Reverse primer | 10 | |
| | 5′-CCGATAGTGATGACCTGACC-3′ | | |
| Rat TNFa | Forward primer | 11 | Ohtomo et al., 2010 |
| | 5′-AACTCGAGACAAGCCCGTAG-3′ | | |
| | Reverse primer | 12 | |
| | 5′-GTACCACCAGTTGGTTGTCTTTGA-3′ | | |
| Rat IL6 | Forward primer | 13 | RealTimePrimers.com |
| | 5′-CTTCCTACCCCAACTTCCAA-3′ | | |
| | Reverse primer | 14 | |
| | 5′-ACCACAGTGAGGAATGTCCA-3′ | | |
| Rat B2m | Forward primer | 15 | RealTimePrimers.com |
| | 5′-TGCTACGTGTCTCAGTTCCA-3′ | | |
| | Reverse primer | 16 | |
| | 5′-GCTCCTTCAGAGTGACGTGT-3′ | | |
| Rat MMP13 | Forward primer | 17 | RealTimePrimers.com |
| | 5′-CCTCTTCTTCTCAGGGAACC-3′ | | |
| | Reverse primer | 18 | |
| | 5′-GGAATTTGTTGGCATGACTC-3′ | | |
| Rat MMP9 | Forward primer | 19 | RealTimePrimers.com |
| | 5′-ACTTCTGGCGTGTGAGTTTC-3′ | | |
| | Reverse primer | 20 | |
| | 5′-TGTATCCGGCAAACTAGCTC-3′ | | |
| Rat MMP2 | Forward primer | 21 | RealTimePrimers.com |
| | 5′-CTTCAGGTTCTCCAGCATGA-3′ | | |
| | Reverse primer | 22 | |
| | 5′-CCGTAAGGGAGACACCAGAT-3′ | | |
| Rat IL-1b | Forward primer | 23 | Nakazawa et al., 2011. |
| | 5′-TCAGGAAGGCAGTGTCACTCATTG-3′ | | |
| | Reverse primer | 24 | |
| Rat ICAM1 | Forward primer | 25 | Ammirante et al., 2010. |
| | 5′-CTGTCAAACGGGAGATGAATGGT-3′ | | |
| | Reverse primer | 26 | |
| | 5′-TCTGGCGGTAATAGGTGTAAATGG-3′ | | |
| Rat MCP1 | Forward primer | 27 | Nakazawa et al., 2006. |
| | 5′-ATGCAGGTCTCTGTCACGCTTCTG-3′ | | |
| | Reverse primer | 28 | |
| | 5′-GACACCTGCTGCTGGTGATTCTCTT-3′ | | |
| Rat E-Selectin | Forward primer | 29 | Hannawa et al., 2005. |
| | 5′-TGCGATGCTGCCTACTTGTG-3′ | | |
| | Reverse primer | 30 | |
| | 5′-AGAGAGTGCCACTACCAAGGGA-3′ | | |
| Rat Ywhaz | Forward primer | 31 | Gubern et al., 2009. |
| | 5′-GATGAAGCCATTGCTGAACTTG-3′ | | |
| | Reverse primer | 32 | |
| | 5′-GTCTCCTTGGGTATCCGATGTC-3′ | | |
| Rat Sdha | Forward primer | 33 | Gubern et al., 2009. |
| | 5′-TCCTTCCCACTGTGCATTACAA-3′ | | |
| | Reverse primer | 34 | |
| | 5′-CGTACAGACCAGGCACAATCTG-3′ | | |

TABLE 3
[ChIP assay primers for SYBR green based real-time PCR],
Related to FIGS. 3E, 3F, and 3G.

| Promoter | Primers | Forward | SEQ ID NO: | Reverse | SEQ ID NO: |
|---|---|---|---|---|---|
| Wnt5a | A1 | 5′-GCATCCCACTACCC | 35 | 5′-GCTGCCTTGACATGGA | 39 |
| Promoter A | | AAGTCC-3′ | | ACCTCA-3′ | |
| | A2 | 5′-CAGCAATAAGTTCC | 36 | 5′-GCTTTGGGGCCACAGA | 40 |
| | | GGGGCG-3′ | | ACAATC-3′ | |
| | A3 | 5′-GCCTCTCCGTGGAA | 37 | 5′-GATGCGCCCAGGAATG | 41 |
| | | CAGTTGC-3′ | | G-3′ | |
| | A4 | 5′-CGCCAGTGCCCGCT | 38 | 5′-CAGCCGAGGAATCCGA | 42 |
| | | TCAG-3′ | | GC-3′ | |

TABLE 4
[TRED and TF bind analysis on the Promoter sequence
(Katula et al., 2012)], Related to FIGS. 3D-3G.

| | | |
|---|---|---|
| Position/Sequence | TRED Score | TFBind Score |

cREL

| | | | |
|---|---|---|---|
| Region 1 | [192 . . . 201] TAGAAATTCC | 3.92 | |
| | [214 . . . 223] CCGGTTTTGC | 2.21 | |
| | [215 . . . 224] CGGTTTTGCC | 3.3 | |
| | 193 (+) SGGRNWTTCC TAGAAATTCC | | 0.819522 |
| | 194 (−) SGGRNWTTCC AGAAATTCCG | | 0.844549 |
| Region 2 | [357 . . . 366] GGGACTTTGC | 5.08 | |
| | 358 (+) SGGRNWTTCC GGGACTTTGC | | 0.854004 |
| Region 3 | [1445 . . . 1454] GCGACTTTCA | 4.12 | |
| Region 4 | [1550 . . . 1559] CGGCATCTCC | 3.3 | |
| | 1565 (−) SGGRNWTTCC GAAAAAGCCA | | 0.850945 |
| Region 5 | [1945 . . . 1954] CCTAATTACC | 1.99 | |
| | 1939 (−) SGGRNWTTCC GGAAAGCCCT | | 0.887097 |
| Region 6 | [2103 . . . 2112] GGGCGCATCC | 2.6 | |
| Region 7 | [2284 . . . 2293] GGCGACTTCC | 3.71 | |
| | 2285 (+) SGGRNWTTCC GGCGACTTCC | | 0.814516 |

p65

| | | | |
|---|---|---|---|
| Region 1 | [192 . . . 201] TAGAAATTCC | 4.17 | |
| | 194 (−) GGGRATTTCC AGAAATTCCG | | 0.868024 |
| Region 2 | [357 . . . 366] GGGACTTTGC | 6.19 | |
| | 358 (+) GGGRATTTCC GGGACTTTGC | | 0.861557 |
| Region 3 | [1445 . . . 1454] GCGACTTTCA | 1.95 | |
| Region 4 | [1550 . . . 1559] CGGCATCTCC | 2.56 | |
| | 1551 (+) GGGRATTTCC CGGCATCTCC | | 0.769102 |
| | 1552 (−) GGAMTTYCC GGCATCTCCC | | 0.803246 |
| Region 5 | [1937 . . . 1946] TGGAAAGCCC | 2.14 | |
| | 1938 (+) GGGRATTTCC TGGAAAGCCC | | 0.782754 |
| | 1939 (−) GGGRATTTCC GGAAAGCCCT | | 0.86491 |
| Region 6 | [2104 . . . 2113] GGCGCATCCC | 1.79 | |
| | 2105 (+) GGGRATTTCC GGCGCATCCC | | 0.765749 |
| Region 7 | [2284 . . . 2293] GGCGACTTCC | 2.72 | |
| | 2285 (+) GGGRATTTCC GGCGACTTCC | | 0.771018 |

NFkB/p50

| | | | |
|---|---|---|---|
| Region 1 | 193 (−) NGGGACTTTCCA TAGAAATTCCGG | | 0.760042 |
| Region 2 | [357 . . . 366] GGGACTTTGC | 5.92 | |
| | 358 (+) GGGGATYCCC GGGACTTTGC | | 0.750555 |
| Region 3 | [1445 . . . 1454] GCGACTTTCA | 0.86 | |
| Region 4 | [1551 . . . 1560] GGCATCTCCC | 1.74 | |
| | 1551 (−) GGGGATYCCC CGGCATCTCC | | 0.790315 |
| | 1552 (−) GGGAMTTYCC GGCATCTCCC | 0.803246 | |
| Region 5 | [1937 . . . 1946] TGGAAAGCCC | 2.08 | |
| | 1939 (−) GGGGATYCCC GGAAAGCCCT | | 0.79498 |
| Region 6 | [2104 . . . 2113] GGCGCATCCC | 3.78 | |
| | 2105 (−) GGGGATYCCC GGCGCATCCC | | 0.75522 |
| Region 7 | [2285 . . . 2294] GCGACTTCCT | 2.95 | |
| | 2285 (+) GGGGATYCCC GGCGACTTCC | | 0.754331 |

TABLE 6
Antibodies

| Antibody | Company | Cat. No |
|---|---|---|
| WNT5A | Thermo Scientific | MA5-15511 |
| FZD5 | EMD Millipore | 06-756 |
| Human Albumin | ABCAM | AB28405 |
| rat Albumin | ABCAM | AB53435 |
| cREL | CST | 12659S |
| GAPDH | EMD Millipore | MAB374 |
| b-actin | Abcam | ab8229 |
| ECL Plex goat-a-rabbit IgG Cy5 | GE-Amersham | PA45011 |
| ECL Plex goat-a-mouse IgG Cy3 | GE-Amersham | PA43010V |

TABLE 5
[CpG islands detected by MethPrimer (Li and Dahiya,
2002). Criteria: Island size >100, GC Percent >50.0,
Obs/Exp >0.6): 5 CpG island(s) were found in the sequence],
Related to FIGURES 3E, 3F, and 3G.

| | Size | (Start-End) |
|---|---|---|
| Island 1 | 175 bp | (137-311) |
| Island 2 | 173 bp | (483-655) |
| Island 3 | 144 bp | (664-807) |
| Island 4 | 793 bp | (948-1740) |
| Island 5 | 338 bp | (1929-2266) |

TABLE 7
Recombinant proteins

| Protein | Company | Cat. No |
|---|---|---|
| h/mWNT5A | R&D systems | 645-WN |
| hWNT3A | R&D systems | 5036-WN |
| PEDF | EMD Millipore | GF134 |
| TNFα | Cell sciences | CSI15659A |
| Basic FGF | Stemgent | 03-0002 |

### REFERENCES CITED IN THIS EXAMPLE

1. Nusse R, Clevers H. Wnt/β-Catenin Signaling, Disease, and Emerging Therapeutic Modalities. Cell. 2017; 169(6):985-999.
2. De A. Wnt/Ca2+ signaling pathway: a brief overview. Acta Biochim Biophys Sin (Shanghai). 2011; 43(10):745-756.
3. Ring L, Neth P, Weber C, Steffens S, Faussner A. β-Catenin-dependent pathway activation by both promiscuous “canonical” WNT3a-, and specific “noncanonical” WNT4- and WNT5a-FZD receptor combinations with strong differences in LRP5 and LRP6 dependency. Cell Signal. 2014; 26(2):260-267.
4. Sen M, Chamorro M, Reifert J, Corr M, Carson D A. Blockade of Wnt-5A/frizzled 5 signaling inhibits rheumatoid synoviocyte activation. Arthritis Rheum. 2001; 44(4):772-781.
5. Rauner M, Stein N, Winzer M, et al. WNT5A is induced by inflammatory mediators in bone marrow stromal cells and regulates cytokine and chemokine production. J Bone Miner Res. 2012; 27(3):575-585.
6. Ackers I, Szymanski C, Duckett K J, McCall K, Malgor R. Wnt5a Signaling in Atherosclerosis, its Effect on OxLDL Uptake and Foam Cell Differentiation. The FASEB Journal. 2015; 29(1_supplement):609.4.
7. Naskar D, Maiti G, Chakraborty A, Roy A, Chattopadhyay D, Sen M. Wnt5a-Rac1-NF-κB homeostatic circuitry sustains innate immune functions in macrophages. J Immunol. 2014; 192(9):4386-4397.
8. Pereira C, Schaer D J, Bachli E B, Kurrer M O, Schoedon G. Wnt5A/CaMKII signaling contributes to the inflammatory response of macrophages and is a target for the antiinflammatory action of activated protein C and interleukin-10. Arterioscler Thromb Vasc Biol. 2008; 28(3):504-510.
9. Zhao Y, Wang C-L, Li R-M, et al. Wnt5a promotes inflammatory responses via nuclear factor κB (NF-κB) and mitogen-activated protein kinase (MAPK) pathways in human dental pulp cells. J Biol Chem. 2014; 289(30):21028-21039.
10. Katula K S, Joyner-Powell N B, Hsu C-C, Kuk A. Differential regulation of the mouse and human Wnt5a alternative promoters A and B. DNA Cell Biol. 2012; 31(11):1585-1597.
11. Mukherjee P K, Marcheselli V L, Serhan C N, Bazan N G. Neuroprotectin D1: a docosahexaenoic acid-derived docosatriene protects human retinal pigment epithelial cells from oxidative stress. Proc Nat Acad Sci USA. 2004; 101(22):8491-8496.
12. Calandria J M, Mukherjee P K, de Rivero Vaccari J C, Zhu M, Petasis N A, Bazan N G. Ataxin-1 Poly(Q)-induced Proteotoxic Stress and Apoptosis Are Attenuated in Neural Cells by Docosahexaenoic Acid-derived Neuroprotectin D1. J Biol Chem. 2012; 287(28):23726-23739.
13. Calandria J M, Marcheselli V L, Mukherjee P K, et al. Selective Survival Rescue in 15-Lipoxygenase-1-deficient Retinal Pigment Epithelial Cells by the Novel Docosahexaenoic Acid-derived Mediator, Neuroprotectin D1. J Biol Chem. 2009; 284(26):17877-17882.
14. Bazan N G. Homeostatic regulation of photoreceptor cell integrity: significance of the potent mediator neuroprotectin D1 biosynthesized from docosahexaenoic acid: the Proctor Lecture. Invest Ophthalmol Vis Sci. 2007; 48(11):4866-4881; biography 4864-4865.
15. Bazan N G. Cell survival matters: docosahexaenoic acid signaling, neuroprotection and photoreceptors. Trends in Neurosciences. 2006; 29(5):263-271.
16. Calandria J M, Asatryan A, Balaszczuk V, et al. NPD1-mediated stereoselective regulation of BIRC3 expression through cREL is decisive for neural cell survival. Cell Death Differ. 2015; 22(8):1363-1377.
17. Belayev L, Khoutorova L, Atkins K D, et al. Docosahexaenoic Acid therapy of experimental ischemic stroke. Transl Stroke Res. 2011; 2(1):33-41.
18. Mukherjee P K, Marcheselli V L, Barreiro S, Hu J, Bok D, Bazan N G. Neurotrophins enhance retinal pigment epithelial cell survival through neuroprotectin D1 signaling. Proc Natl Acad Sci USA. 2007; 104(32):13152-13157.
19. Jenei V, Sherwood V, Howlin J, et al. A t-butyloxycarbonyl-modified Wnt5a-derived hexapeptide functions as a potent antagonist of Wnt5a-dependent melanoma cell invasion. Proc Natl Acad Sci USA. 2009; 106(46):19473-19478.
20. Mikels A J, Nusse R. Purified Wnt5a protein activates or inhibits beta-catenin-TCF signaling depending on receptor context. PLoS Biol. 2006; 4(4):e115.
21. Sato A, Kayama H, Shojima K, et al. The Wnt5a-Ror2 axis promotes the signaling circuit between interleukin-12 and interferon-y in colitis. Sci Rep. 2015; 5:10536.
22. Vaidya H, Rumph C, Katula K S. Inactivation of the WNT5A Alternative Promoter B Is Associated with DNA Methylation and Histone Modification in Osteosarcoma Cell Lines U2OS and SaOS-2. PLoS ONE. 2016; 11(3):e0151392.
23. Gross J C, Chaudhary V, Bartscherer K, Boutros M. Active Wnt proteins are secreted on exosomes. Nat Cell Biol. 2012; 14(10):1036-1045.
24. Willert K, Nusse R. Wnt proteins. Cold Spring Harb Perspect Biol. 2012; 4(9):a007864.
25. Chen W, ten Berge D, Brown J, et al. Disheveled 2 recruits beta-arrestin 2 to mediate Wnt5A-stimulated endocytosis of Frizzled 4. Science. 2003; 301(5638):1391-1394.
26. Feng Q, Gao N. Keeping Wnt signalosome in check by vesicular traffic. J Cell Physiol. 2015; 230(6):1170-1180.
27. Dutta D, Williamson C D, Cole N B, Donaldson J G. Pitstop 2 is a potent inhibitor of clathrin-independent endocytosis. PLoS ONE. 2012; 7(9):e45799.
28. Belayev L, Mukherjee P K, Balaszczuk V, et al. Neuroprotectin D1 upregulates Iduna expression and provides protection in cellular uncompensated oxidative stress and in experimental ischemic stroke. Cell Death Differ. 2017; 24(6):1091-1099.
29. Marcheselli V L, Hong S, Lukiw W J, et al. Novel Docosanoids Inhibit Brain Ischemia-Reperfusion-mediated Leukocyte Infiltration and Pro-inflammatory Gene Expression. J Biol Chem. 2003; 278(44):43807-43817.
30. Belayev L, Lu Y, Bazan N G. Chapter 35—Brain Ischemia and Reperfusion: Cellular and Molecular Mechanisms in Stroke Injury. In: Brady S T, Siegel G J, Albers R W, Price D L, eds. Basic Neurochemistry (Eighth Edition). New York: Academic Press; 2012:621-642.
31. Buga A-M, Sascau M, Pisoschi C, Herndon J G, Kessler C, Popa-Wagner A. The genomic response of the ipsilateral and contralateral cortex to stroke in aged rats. J Cell Mol Med. 2008; 12(6b):2731-2753.
32. Yamagata K, Li X, Ikegaki S, et al. Dissection of Wnt5a-Ror2 signaling leading to matrix metalloproteinase (MMP-13) expression. J Biol Chem. 2012; 287(2):1588-1599.
33. Kim J, Kim J, Kim D W, et al. Wnt5a induces endothelial inflammation via beta-catenin-independent signaling. J Immunol. 2010; 185(2):1274-1282.
34. Diaz-Munoz M D, Osma-Garcia I C, Fresno M, Iniguez M A. Involvement of PGE2 and the cAMP signalling pathway in the up-regulation of COX-2 and mPGES-1 expression in LPS-activated macrophages. Biochem J. 2012; 443(2):451-461.
35. Nishita M, Enomoto M, Yamagata K, Minami Y. Cell/tissue-tropic functions of Wnt5a signaling in normal and cancer cells. Trends Cell Biol. 2010; 20(6):346-354.
36. Mercau M E, Astort F, Giordanino E F, et al. Involvement of PI3K/Akt and p38 MAPK in the induction of COX-2 expression by bacterial lipopolysaccharide in murine adrenocortical cells. Mol Cell Endocrinol. 2014; 384(1-2):43-51.
37. Asatryan A, Bazan N G. Molecular mechanisms of signaling via the docosanoid neuroprotectin D1 for cellular homeostasis and neuroprotection. J Biol Chem. 2017; 292(30):12390-12397.
38. Shojima K, Sato A, Hanaki H, et al. Wnt5a promotes cancer cell invasion and proliferation by receptor-mediated endocytosis-dependent and -independent mechanisms, respectively. Sci Rep. 2015; 5:8042.
39. Kurayoshi M, Yamamoto H, Izumi S, Kikuchi A. Post-translational palmitoylation and glycosylation of Wnt-5a are necessary for its signalling. Biochem J. 2007; 402(3):515-523.
40. Shimizu T, Smits R, Ikenaka K. Microglia-induced activation of non-canonical Wnt signaling aggravates neurodegeneration in demyelinating disorders. Mol Cell Biol. August 2016.
41. Sessa R, Yuen D, Wan S, et al. Monocyte-derived Wnt5a regulates inflammatory lymphangiogenesis. Cell Res. 2016; 26(2):262-265.
42. Korn C, Scholz B, Hu J, et al. Endothelial cell-derived non-canonical Wnt ligands control vascular pruning in angiogenesis. Development. 2014; 141(8):1757-1766.
43. Skaria T, Bachli E, Schoedon G. Wnt5A/Ryk signaling critically affects barrier function in human vascular endothelial cells. Cell Adh Migr. 2017; 11(1):24-38.
44. Li Y, Li B, Wan X, Zhang W, Zhong L, Tang S-J. NMDA receptor activation stimulates transcription-independent rapid wnt5a protein synthesis via the MAPK signaling pathway. Mol Brain. 2012; 5:1.
45. Kankaanpaa P, Paavolainen L, Tiitta S, et al. BioImageXD: an open, general-purpose and high-throughput image-processing platform. Nat Methods. 2012; 9(7):683-689.
46. Schneider C A, Rasband W S, Eliceiri K W. NIH Image to ImageJ: 25 years of image analysis. Nat Methods. 2012; 9(7):671-675.
47. Friedman D B. Quantitative proteomics for two-dimensional gels using difference gel electrophoresis. Methods Mol Biol. 2007; 367:219-239.
48. Veeman M T, Slusarski D C, Kaykas A, Louie S H, Moon R T. Zebrafish prickle, a modulator of noncanonical Wnt/Fz signaling, regulates gastrulation movements. Curr Biol. 2003; 13(8):680-685.
49. Jiang C, Xuan Z, Zhao F, Zhang M Q. TRED: a transcriptional regulatory element database, new entries and other development. Nucleic Acids Res. 2007; 35(Database issue):D137-140.
50. Tsunoda T, Takagi T. Estimating transcription factor bindability on DNA. Bioinformatics. 1999; 15(7-8):622-630.
51. Li L-C, Dahiya R. MethPrimer: designing primers for methylation PCRs. Bioinformatics. 2002; 18(11):1427-1431. Ye, J. et al. Primer-BLAST: a tool to design target-specific primers for polymerase chain reaction. BMC Bioinformatics 13, 134 (2012).
52. Obenaus A, Dilmac N, Tone B, et al. Long-term magnetic resonance imaging of stem cells in neonatal ischemic injury. Ann Neurol. 2011; 69(2):282-291.

### Example 3

cRel and Wnt5a Frizzled 5 Receptor-Mediated Inflammatory Regulation Reveals Dual Targets for Neuroprotection

Wnt5a is engaged in a multitude of cell signaling processes. Here, we demonstrate that Wnt5a triggers inflammatory responses via NFkB/p65 and damage in retinal pigment epithelial (RPE) cells undergoing uncompensated oxidative stress (UOS) and in ischemic stroke. We found that Wnt5a Clathrin-mediated uptake leads to NFkB/p65 activation. Wnt5a is secreted in an exosome-independent fashion. Docosahexaenoic acid (DHA) and its derivative, Neuroprotectin D1 (NPD1), upregulate c-Rel expression that blunts Wnt5a abundance by competing with NFkB/p65 in the Wnt5a promoter A. Wnt5a increases in ischemic stroke penumbra and blood, while DHA reduces Wnt5a abundance with concomitant neuroprotection. Peptide inhibitor of Wnt5a binding, Box5, is also neuroprotective. DHA-decreased Wnt5a expression is concurrent with a drop in NFkB-driven inflammatory cytokines expression, uncovering mechanisms after stroke, as in RPE cells exposed to UOS. Limiting the Wnt5a activity via Box5 reduces stroke size, indicating neuroprotection sites pertinent to onset and progression of retinal degenerations and stroke consequences.

Wnt signaling pathways are associated with normal functions and pathology, including development and cancer (Nusse and Clevers, 2017). From the wingless family of ligands, Wnt5a is a secretory lipid-modified glycoprotein that, in certain cases, activates calcium-dependent signaling via interaction with Frizzled proteins, Ror1/2, RYK, and RTK (De, 2011). However, Wnt5a activity is driven by cellular context and is also able to activate beta-catenin via promiscuous interaction with LRP5/6 (Ring et al., 2014). During tissue morphogenesis and differentiation, Wnt5a is involved in synaptogenesis (Varela-Nallar et al., 2010) via Ca++ dependent signaling. Wnt5a has also been associated with inflammatory diseases like rheumatoid arthritis (Rauner et al., 2012; Sen et al., 2001) and atherosclerosis (Ackers et al., 2015). Moreover, Wnt5a is released by macrophages (Naskar et al., 2014; Pereira et al., 2008) to activate NFkB (Zhao et al., 2014). Because NFkB/p65 is a Wnt5a transcriptional activator (Katula et al., 2012), it heightens its own expression. Thus, we asked if the modulators of uncompensated oxidative stress (UOS) and cell survival (Calandria et al., 2012; Mukherjee et al., 2004), DHA/NPD1, regulate Wnt5a expression and its availability. Using human primary retinal pigment epithelial (hpRPE) cells, we have addressed events relevant to retinal degenerations by activating NPD1 synthesis from DHA (Bazan, 2006, 2007; Calandria et al., 2009) since these cells support photoreceptor integrity. In these cells, we show that cREL mediates Wnt5a transcriptional regulation by NPD1. In brain ischemia reperfusion, DHA fosters neuronal survival via NPD1 synthesis that in turn activates NFkB/cRel (Calandria et al., 2015). We provide evidence that Wnt5a is upregulated in stroke penumbra and augmented in the bloodstream, favoring activation of immune cells and their recruitment into damaged brain. In addition, DHA decreased bloodstream and penumbra Wnt5a abundance, leading to neuroprotection. Altogether, these results indicate inflammatory modulatory signaling mediated by DHA/NPD1 engages Wnt5a in responses to neural cell injury.

Docosanoids Inhibit UOS-Triggered WNT5a and Fzd5 Transcription with Concomitant Reduction in Apoptosis

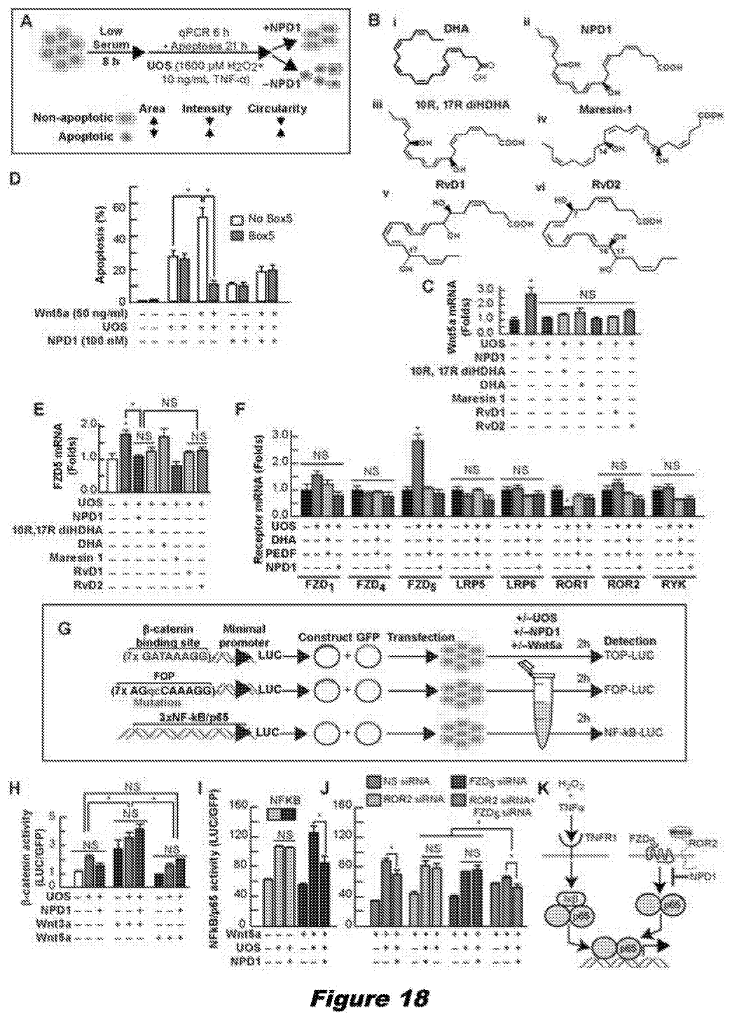

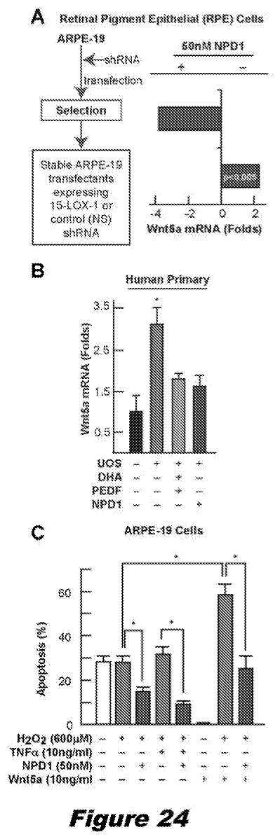

Observations showed that UOS induced by H₂O₂ plus TNFα triggers NPD1 synthesis via 15-lipoxygenase-1 (15-LOX-1) in RPE cells and the silencing of this enzyme results in NPD1 depletion (Calandria et al., 2009). Six hours after the initiation of UOS, 15-LOX-1 deficient cells display a 2-fold increase in Wnt5a expression (FIG. 24A) that was brought down to below controls by NPD1. Also, DHA plus pigment epithelium-derived factor (PEDF), a neurotrophin agonist of NPD1 synthesis (Mukherjee et al., 2007), prevented Wnt5a upregulation in hpRPE cells (FIG. 24B). To test the idea that other docosanoids (FIG. 18Bi-vi) downregulated Wnt5a transcription, we confronted hpRPE cells for 6 hours using 1,600 μM H₂O₂ and 10 ng/ml TNFα (FIG. 18A) in the presence or absence of DHA (FIG. 18Bi), NPD1 (FIG. 18Cii), 10R, 17R diHDHA (FIG. 18Biii), Maresin-1 (FIG. 18Biv), RvD1 (FIG. 18Bv) or RvD2 (FIG. 18Biv). All docosanoids decreased the expression of Wnt5a to control levels (FIG. 18C). Recombinant Wnt5a potentiated cell death by UOS in ARPE-19 cells (FIG. 24C) and in hpRPE (FIG. 18D). A hexapeptide that corresponds to the amino acid portion 332 to 337 of Wnt5a with the t-Boc substitution in the N-terminal (t-Boc-NH-Met-Asp-Gly-Cys-Glu-Leu-CO₂H (SEQ ID NO: 1)), Box5 is a Wnt ligand analog that blocks binding to receptors (Jenei et al., 2009). Box5 or NPD1 hindered apoptosis by UOS in the presence of the Wnt ligand. Wnt5a alone had no effect on RPE cells (FIG. 24C), indicating that the Wnt ligand enhanced apoptosis in susceptible cells undergoing UOS but not in resting cells.

To assess the co-regulation of Wnt5a receptors/co-receptors (FZD4 and 5, LRP5/6, RYK, and ROR1/2) (Mikels and Nusse, 2006), we assayed their expression by SYBR green-based real-time PCR. hpRPE were exposed to UOS, FZD5 mRNA rose 2-fold and then dropped to control levels when docosanoids were added (FIG. 18E). DHA leads to the synthesis of 10R, 17R diHDHA, Maresin 1, RVD1, RVD2, and NPD1 (FIG. 18B i-vi), however, this fatty acid alone did not affect FZD5 expression, indicating that its conversion to lipid mediators is required to counteract the effect of UOS on FZD5. Unlike FZD5, other Wnt signaling receptors: FZD1, FZD4, LRP5, LRP6, ROR2, and RYK, remained unchanged, although ROR1 expression was reduced under UOS and re-established by NPD1 (FIG. 18F). These results also advocate that FZD5 is linked to Wnt5a signaling in RPE cells undergoing UOS.

WNT5a-Dependent Activation of NFkB Requires FZD5 and ROR2

Wnt5a elicits positive and negative actions on β-catenin activity, depending on the receptor context and the presence of other Wnt ligands (Mikels and Nusse, 2006). We tested whether or not Wnt5a by itself activates β-catenin using TOP Flash/FOP flash reporter system in hpRPE cells undergoing UOS+/−NPD1 (FIG. 18G). In the absence of Wnt5a or Wnt3a, β-catenin activity was not significantly altered by UOS or NPD1 (FIG. 18H). When Wnt3a was added, luciferase rose almost twice, which is consistent with the β-catenin co-activation of TCF/LEF reporter system. Wnt5a alone did not affect β-catenin (FIG. 18H), indicating that, in this case, Wnt5a signaling in the absence of activated β-catenin does not involve TCF/LEF-related gene expression.

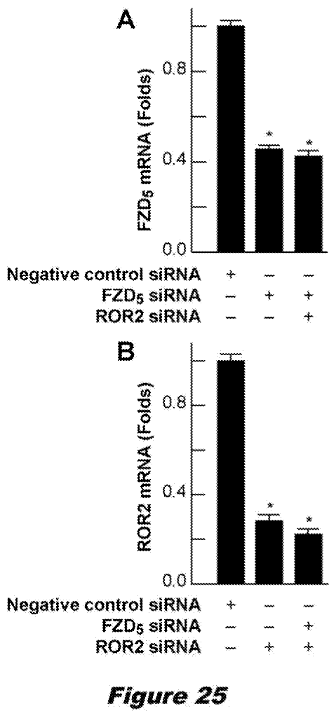

Without wishing to be bound by theory, Wnt5a interacts with FZD5 and ROR2 to trigger inflammatory gene expression via NFkB activation (Naskar et al., 2014; Sato et al., 2015). To determine if Wnt5a activates NFkB, hpRPE were transfected with a construct that encompassed 3 p65 high-affinity binding sites in tandem, driving the expression of the luciferase reporter gene (FIG. 18G). hpRPE cells exposed to UOS showed increased luciferase activity, and NPD1 did not affect NFkB/p65 as shown previously (FIG. 18I) (Calandria et al., 2015). Exposure of 2 hours of Wnt5a heightened NFkB activation in the presence of UOS, and NPD1 reduced luciferase activity (FIG. 18I). These results indicate that the activation of NFkB/p65 via Wnt5a is exerted by a different signaling pathway than the one triggered by UOS/TNFα, and in this case, the effect is responsive to NPD1 (FIG. 18K). To assess whether or not FDZ5 and ROR2 were involved in NFkB activation by Wnt5a, cells were co-transfected with siRNAs targeting the 2 receptors (FIG. 25). hpRPE cells co-transfected with control siRNA showed the same pattern of NFkB activity seen in FIG. 1I. ROR2 or FZD5 siRNAs separately abolished the difference between UOS and UOS+NPD1 (FIG. 18J). The co-transfection of both siRNAs, against ROR2 and FZD5 together, induced a higher NFkB activation that was not affected by NPD1. When Wnt5a was added to the double knockdown cells, the NFkB activity did not differ from controls, but it did show a slight yet significant difference with the UOS-treated cells, indicating both receptor and co-receptor are required to activate NFkB/p65.

NPD1 Stimulates cRel Binding to Wnt5a Promoter a that in Turn Prevents Transcriptional Activation

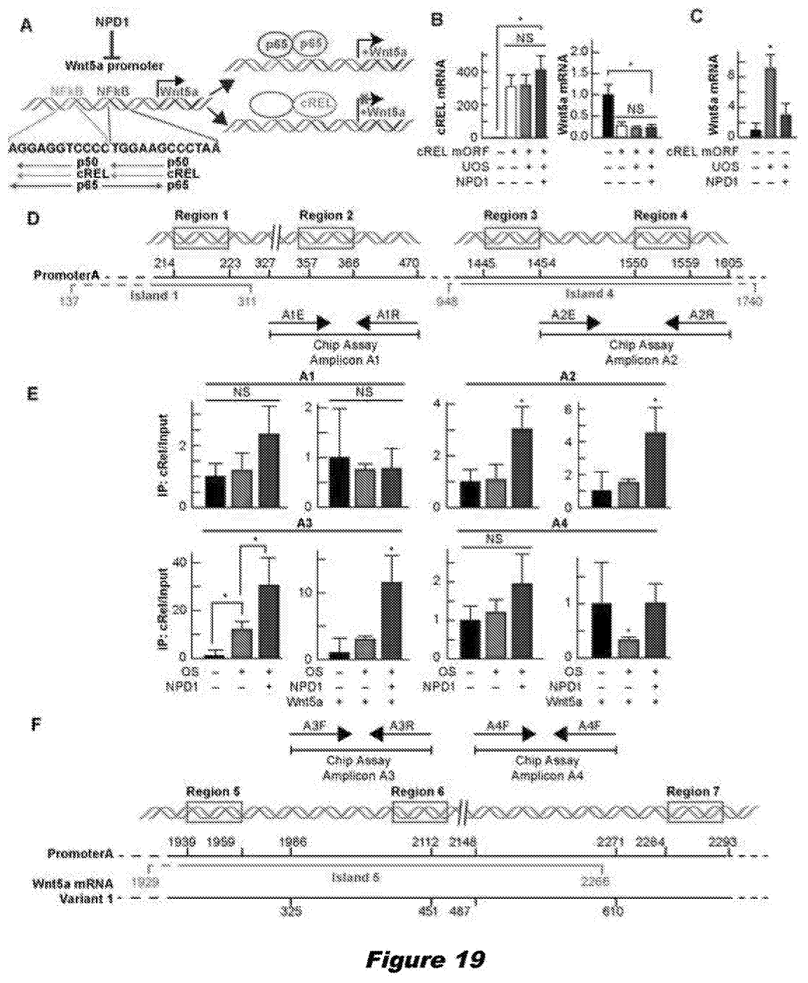

The Wnt5a gene is under the regulation of 2 promoters located in exon1a and exon2 (Vaidya et al., 2016). Promoter A drives the expression of the largest form, variant-1, and contains at least 2 binding sites for NFkB (Katula et al., 2012). In-silico analysis using TRED software showed that both sites have a high affinity for 3 NFkB members: p50, cRel, and p65. Downstream, p50 and cRel binding are opposed to p65 site, indicating that p65 and cRel activity compete to oppose each other (FIG. 19A and Table S1). To confirm a direct link between cRel expression and Wnt5a transcription, we over-expressed the transcription factor in hpRPE (FIG. 19B). cRel overexpression decreased Wnt5a mRNA (FIG. 19B). The increase in cRel availability dominantly shut off Wnt5a expression regardless of the treatment. Altogether, the data indicate that cRel blocks Wnt5a expression triggered by UOS, which may be a key modulatory NPD1 function.

Based on the regions found to bind NFkB members (Table S1), we designed 4 sets of primers that bound in the proximity or within the regions of interest to perform SYBR green-based real-time PCR detection (FIG. 19D-F and Table S3). FIGS. 19D and F and Table S2 also show regions of high probability for methylation, a mechanism to block Wnt5a transcription (Vaidya et al., 2016). Micrococcal DNase digested genomic DNA fragments from UOS or UOS-plus-NPD1-treated RPE cells, +/−rWnt5a, were pulled down by a cRel antibody and used to test the 4 sets of primers. Amplicon A1, localized close to Region 1 and overlap to Region 2 cRel/p65 binding sites (FIG. 19D), showed no differences between treatments, indicating no differential binding of cRel to the genomic DNA in the presence of Wnt5a and NPD1 (FIG. 19E). Methylation of Region 1 may occur since it was predicted by the Methprimer software (Table S5). The amplicon 2 encompassing Region 3 and 4 displayed twice the amount of cRel bound to genomic DNA under NPD1 treatment. Within the proximity of Region 5 and overlaps Region 6 (FIG. 19F), the amplicon 3 displayed the largest differences between treatments, reaching more than 20-fold when NPD1 was added to RPE cells undergoing UOS in the absence of Wnt5a and 10-fold when Wnt5a was present. The results obtained for amplicon 3 indicate competition between the Wnt5a-activated p65 and NPD1-activated cRel (FIG. 19E). Finally, Amplicon 4, which is located upstream of Region 7, showed no differences in the absence of Wnt5a but did show a decrease in RPE cells undergoing UOS in the presence of rWnt5a. NPD1 restored the cRel binding to control levels, indicating that in the presence of NPD1, cRel displaces the initially bound p65 (FIGS. 19E and F). These data indicate that there is a binding interactive competition between p65 and cRel that depends on availability and other factors, such as methylation for NPD1-mediated cell survival.

Extracellular Availability of Wnt5a is Mainly Endosome-Free and is Controlled by NPD1

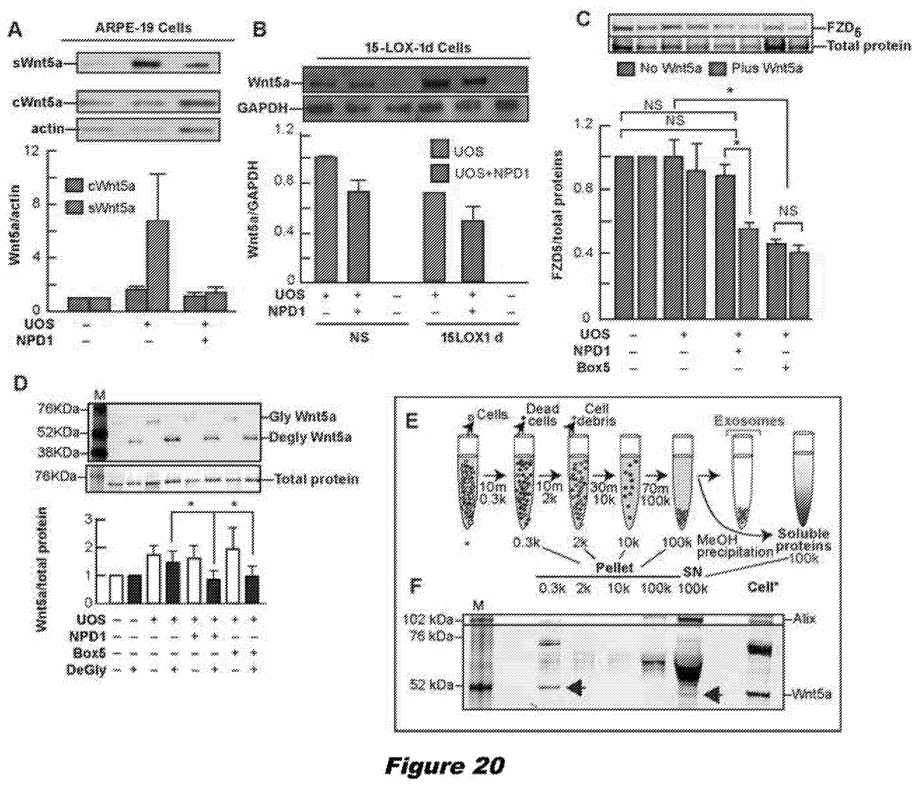

Western blot assays using cellular lysates and medium (precipitated by Methanol/Chloroform to bring down secreted Wnt5a) from hpRPE undergoing UOS+/−NPD1 to assess Wnt5a release were used. We found that UOS induced an increase in secreted Wnt5a in ARPE-19 cells (FIG. 20A). NPD1 addition decreases band intensity close to control levels (FIG. 20A). Wnt5a cellular abundance remained constant for all the treatments in cells including 15-LOX-1d cells, indicating a tight regulation of the balance between secreted (sWnt5a) and cellular Wnt5a (cWnt5a) (FIGS. 20A and B).

Frizzled 5 mRNA expression was found to be upregulated by UOS in hpRPE cells and downregulated by NPD1. To determine the FZD5 protein availability, a western blot assay was performed in cell extracts from hpRPE cells exposed to UOS 1+/−NPD1 or Box5 when Wnt5a was added. Contrary to the levels of mRNA (FIG. 18E), cells exposed to UOS displayed a similar FZD5 content than controls, indicating receptor regulation by degradation. The presence of Box5 decreased FZD5 abundance by half of the level, even in the presence of Wnt5a, showing that the inhibition of the binding of the true ligand enhances receptor degradation as well. Intriguingly, when NPD1 was added in the absence of Wnt5a, no differences in the FZD5 receptor were observed although, when the ligand was present, a steep decrease in the receptor content was evident (FIG. 20C), indicating that NPD1 enhances FZD5 degradation via internalization of the FZD5/Wnt5a complex.

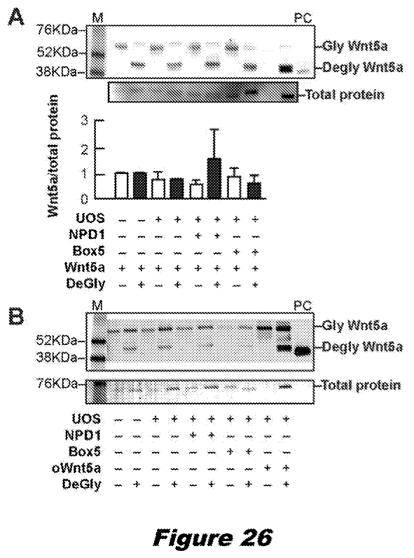

Wnt5a display in western blots, besides the light band that ran between 52 and 38 KDa, a higher molecular mass band with all the antibodies used in this study. Since Wnt5a is glycosylated and palmitoylated (Kurayoshi et al., 2007), we tested if the band above 52 KDa was a highly glycosylated form of the ligand by total deglycosylation (Degly) of the precipitated medium in comparison non-deglycosylated samples (Gly). Deglycosylation followed the patterned of intensity observed when the cells were exposed to UOS+/−NPD1 or Box5 (FIG. 20D), however, the samples that were not digested showed a different pattern due to different affinity of the antibodies to the modified Wnt5a. Overexpression of Wnt5a showed that Wnt5a increases after deglycosylation, but not noticeable change in the hyper glycosylated state by the antibody (FIG. 26), indicating that hyper glycosylated secreted protein may be a mechanism by which the cell modulates ligand activity.

Active Wnt5a can be released in exosomes (Gross et al., 2012). To address whether Wnt5a was released by hpRPE cells in exosomes we performed medium ultracentrifugation from cells undergoing UOS for 10 hours (FIG. 20E). Western blot of the first pellet after spinning at 300 rpm (dead cells pulled-down), showed a 35 KDa band as that of mature Wnt5a. The bands pattern closely resembles the one in whole cells (FIG. 20F). Pellets of 2,000 and 10,000 rpm, containing cell debris (FIG. 20E), displayed no bands and the pellet from ultracentrifugation at 100,000 rpm (exosomes) lack a 42 KDa band. The supernatant was then precipitated using Methanol/Chloroform and a 42 KDa emerged, indicating that most of Wnt5a is not contained in exosomes. However, a band above 52 KDa appeared in the pellet obtained in the 100K×g centrifugation and a very intense band in the supernatant that was further precipitated, indicating that the glycosylated forms were present in exosomes, but mainly in soluble manner. These results indicated that Wnt5a is mainly release in an exosome-free manner by hpRPE cells undergoing UOS.

Wnt5a/FZD5 Clathrin-Mediated Endocytosis is Required for NFkB Activation.

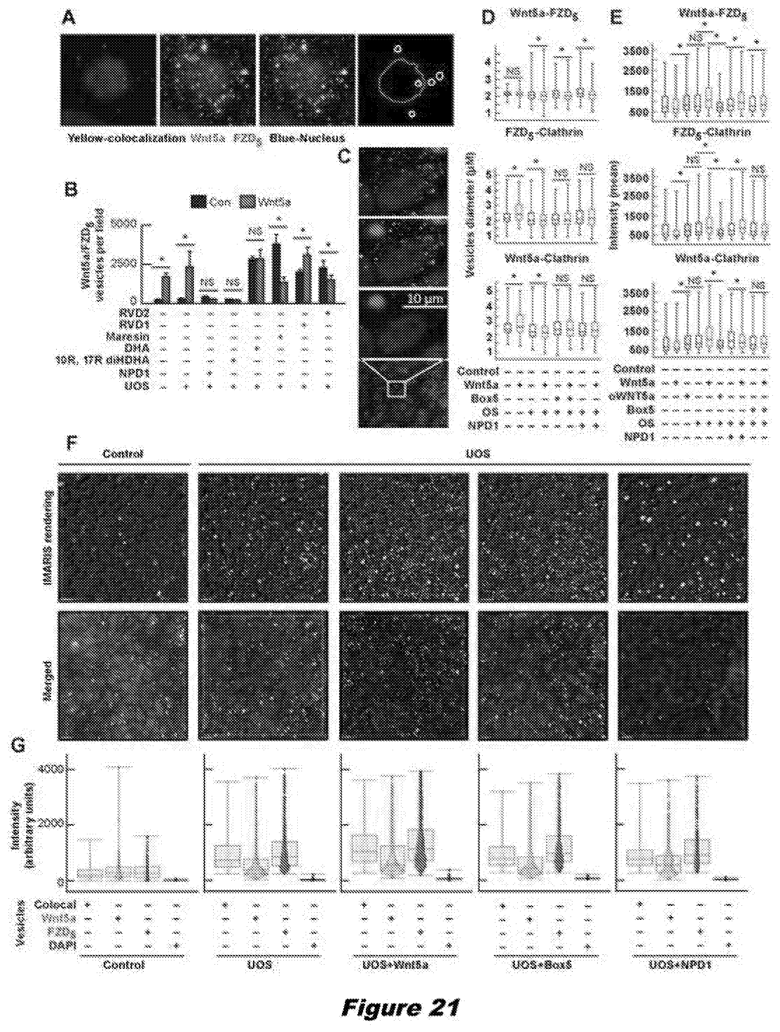

Wnt5a is processed to maturity in the ER/Golgi where it binds to its receptor and join the recycled protein pool (Willert and Nusse, 2012). To ascertain localization and interaction between FZD5 and Wnt5a, we incubated hpRPE undergoing UOS with Wnt5a+/−NPD1 or other lipid mediators for 2 hours. Immunocytochemistry in detergent-permeabilized cells was analyzed by BioImageXD to assess colocalization of FZD5/Wnt5a signal and by ImageJ or IMARIS software to count colocalized objects in each field. The mean of vesicles/field that showed colocalization of FZD5/Wnt5a was increased with the addition of the ligand in the control and UOS cells (FIG. 21A-C), indicating that the Wnt ligand promotes its own internalization. NPD1 and its bioactive stereoisomer 10R, 17R diHDHA decreased the amount of vesicles per field in the presence or absence of Wnt5a, but not the other docosanoids and related bioactive lipids tested (FIG. 21i).

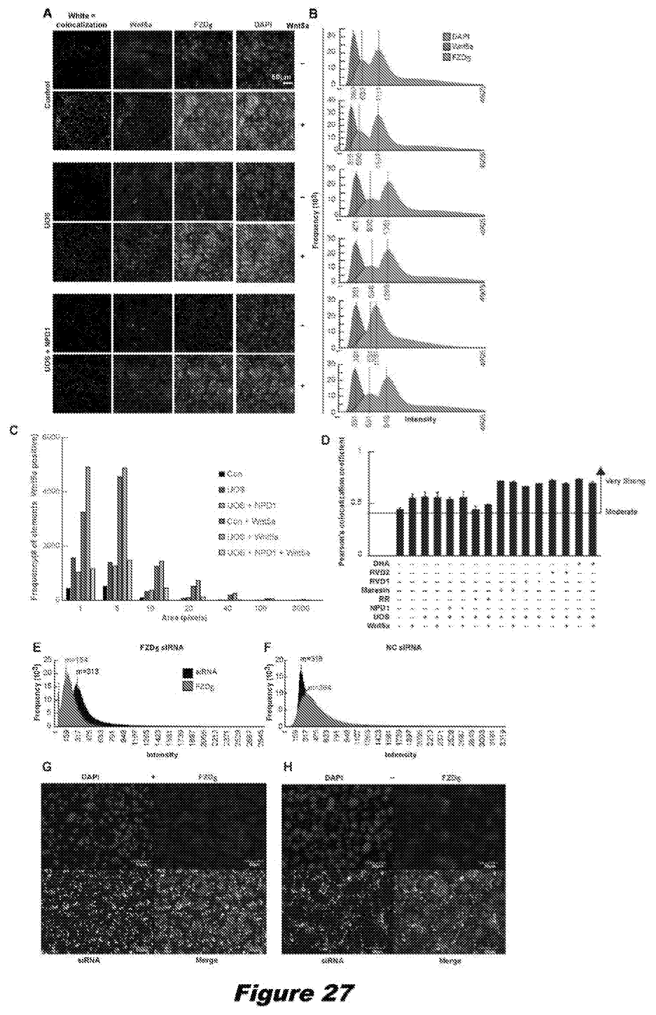

The population of the FZD5/Wnt5a vesicles showed various amplitudes of sizes (FIG. 21A, C, D and FIG. 27C). The addition of Wnt5a induced an increase in the size of vesicles containing FZD5 receptor/Clathrin and, Wnt5a/Clathrin in resting cells reflecting an increase in FZD5 and Wnt5a internalization. However, the vesicles that showed colocalization between the FZD5 and Wnt5a remained unchanged in control cells, indicating that at least part of the Wnt5a uptake may be mediated by other receptors in resting cells (FIG. 21 D). Conversely, under UOS conditions, the size of the vesicles colocalizing the signals FZD5/Wnt5a, FZD5/Clathrin and Wnt5a/Clathrin decreased with the addition of Wnt5a to the medium, showing the same trend and thus indicating that under oxidative stress conditions Wnt5a and FZD5 may be internalized together via clathrin. Neither NPD1 nor Box5 affected the magnitude of the Wnt5a effect on the size of FZD5/Wnt5a vesicles under UOS conditions. FZD5/Clathrin and Wnt5a/Clathrin vesicle sizes remained unchanged with the addition of Wnt5a to UOS cells+/−NPD1 or Box5 (FIG. 21 D). Together, the variation in vesicle size points to a complex mechanism of uptake of Wnt5a that involves clathrin, FZD5 and/or other receptors.

To determine vesicles load, we used the mean of the intensity as an indicator for each vesicle. Furthermore, to assess the role of enhanced expression Wnt5a on the load of the vesicles, we overexpressed variant 1 of the human Wnt5a and subjected them, along with control hpRPE cells, to UOS. The load of the FZD5/clathrin, Wnt5a/clathrin and Wnt5a/FZD5 vesicles in control cells overexpressing and exposed to external Wnt5a vs control showed the same pattern; the control vesicles did not differ in the load between overexpressing the ligand and cells exposed to external Wnt5a. However, cells that overexpressed Wnt5a depicted more intensity than those exposed to external Wnt5a for the 3 types of colocalization observed, indicating that increased Wnt5a expression triggers enhanced upload of FZD5 and Wnt5a in the vesicle. Moreover, Wnt5a overexpression mimicked the intensity observed in the UOS-treated cells for the 3 types of colocalization in agreement with the consequences of oxidative stress. The addition of Wnt5a raised the intensity of the vesicles as well. External Wnt5a did not affected vesicle loads that showed FZD5/clathrin or Wnt5a/clathrin signal in the presence of Box5 under UOS but did affected those in which Wnt5a and FZD5 colocalized (FIG. 21 E). In addition, cells exposed to UOS and NPD1 displayed higher Wnt5a/FZD5 and FZD5/Clathrin vesicle intensity when Wnt5a was added, but Wnt5a/Clathrin remained unaffected, indicating that the internalization of Wnt5a have proceed via FZD5/Clathrin (FIG. 21 E).

To assess the vesicles that were positive for Wnt5a, FZD5 or both, we analyzed the confocal z-stacked pictures for control and UOS alone or treated with Wnt5a Box5 and NPD1 (FIG. 21 F upper panels depicts the IMARIS rendering of the actual flattened Z-stack picture in lower panels). UOS increased the number of Wnt5a- and FZD5-positive vesicles compared to control cells. While Box5 and NPD1 decrease the number of FZD5-positive vesicles, the addition of Wnt5a raised the number and the mean intensity of vesicles showing Wnt5a signal in agreement with FIG. 21 B. Box5 did not modify the increase in intensity and number induced by UOS, however, Wnt5a-positive vesicles displayed a difference in mean intensity distribution when NPD1 was added.

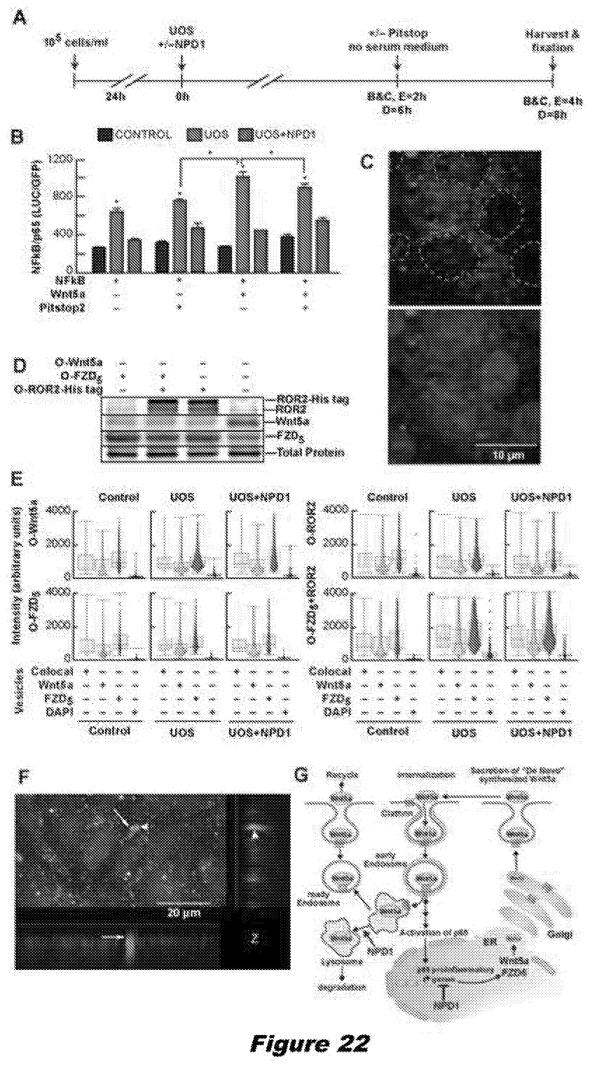

Wnt5a signaling required FZD5 and ROR2 to activate p65/NFkB under UOS conditions (FIG. 18J). The pattern of colocalization between FZD5/Clathrin, Wnt5a/Clathrin and Wnt5a/FZD5 (FIGS. 21 D and E) are compatible with Wnt5a/FZD5 internalization mediated by Clathrin- or Clathrin-mediated endocytosis (CME) (Feng and Gao, 2015). Thus, we asked if sWnt5a plus FZD5 internalization is linked to p65/NFkB activation when hpRPE cells were transfected with the NFkB/p65 reporter construct with UOS+/−200 nM NPD1 plus 50 ng/ml rWnt5a (FIG. 22A). We found that the addition of Pitstop2 in cells undergoing UOS in the presence of Wnt5a decreases the activation of p65/NFkB, indicating that the internalized Wnt5a is responsible for NFkB activation (FIGS. 22B and C).

Wnt5a overexpression disclosed a high number of vesicles that colocalize with FZD5/Wnt5a signal as well as with Wnt5a- and FZD5-positive vesicles, even in the presence of NPD1. FZD5 overexpression display low number of FZD5 vesicles while the overexpression of ROR-2 induced a steep increase in the Wnt5a/FZD5- and Wnt5a-positive vesicles in controls and NPD1-treated cells, but more so in cells undergoing UOS (FIG. 22D). The co-expression of ROR-2 and FZD5 increased the number and intensity of the expression of the 2 genes separately, supporting the observation in FIG. 18G, which shows FZD5, ROR2 and Wnt5a signal together to activate NFkB.

Without wishing to be bound by theory, the heterogeneity of the signal within the vesicles, the size and the intensity observed by immunocytochemistry (FIG. 22F) indicate that NPD1 enhances internalized Wnt5a degradation by favoring the conversion of early endosome to lysosome pathway. Large vesicles that were labeled positive for Wnt5a are shown (FIG. 22G). By increasing Wnt5a degradation, NPD1 can induce a decrease in Wnt5a-triggered NFkB induction independently of cRel activation. Altogether, the results agree with the mechanism indicated in FIG. 22H.

Ischemic Stroke Activates Wnt5a Expression: DHA and Box5 Elicit Neuroprotection

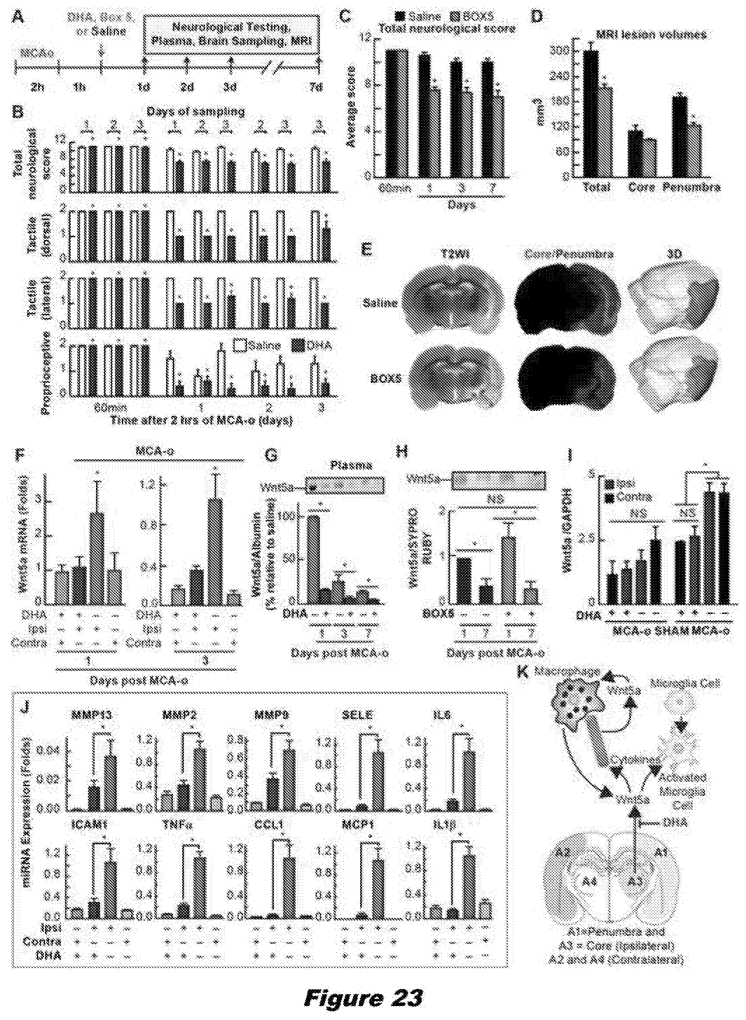

Intravenously (IV) DHA reduces ischemia-reperfusion (I-R) brain damage. To test whether IR increases Wnt5a secretion and signaling, we induced stroke in rats by middle cerebral artery occlusion (MCAo) for 2 hours and, 1 hour later, injected IV saline (vehicle) or DHA. Neurological scores, tactile and proprioceptive tests 1, 2, 3, and 7 days after MCAo showed severe neurological impairments in saline-treated rats (FIGS. 23A and B). DHA treatment improved neurologic scores, including tactile (dorsal and lateral) and proprioceptive forelimb placing reaction (FIG. 23B). To test whether Wnt5a is involved in post ischemia-reperfusion damage, we injected the receptor blocker Box5 after MCAo (FIG. 23A) and found neurological protection remarkable similar to that obtained by DHA treatment (FIG. 23D). Moreover, MRI illustrated decreased volume of brain damage (FIGS. 23 D and E) resembling those observed by DHA injection (Belayev et al., 2017). Furthermore, we assessed Wnt5a mRNA in A1 penumbra, A3 stroke core, and as control A2 and A4 that correspond to contralateral parts of A1 and A3 (FIG. 23K). The penumbra, an area surrounding the ischemic core, is subject to moderate damage and may survive the ischemic reperfusion when treatment is applied. We found that the Wnt5a mRNA was increased in the ipsilateral hemisphere 1-3 days post-surgery and that DHA blocked this surge (FIG. 23F). After stroke, both hemispheres work synergistically to overcome damage (Buga et al., 2008). In this case, the increase in Wnt5a mRNA level was detected only in the ipsilateral hemisphere; the contralateral showed no surge in Wnt5a expression 1-3 days after surgery, indicating a local induction of mRNA expression. However, Wnt5a protein abundance showed that the levels of ipsilateral and contralateral hemispheres A1 and A2 did not differ from one another, and they were both high in saline and low in DHA-treated animals (FIG. 23I). In addition, Wnt5a was enhanced in blood after MCAo 1 day post-stroke and was decreased at day 3 (FIG. 23G). Impairment of the interaction Wnt5a/receptor with Box5 that induced changes in the size of the infarct observed in the MRI (FIGS. 23D and E) and an improvement in neurological score (FIG. 23C) failed to reduce the plasma Wnt5a content, indicating a difference in the action between DHA and Box5. In agreement with the activation of NFkB/p65, the expression of NFkB-activated inflammation mediators IL6, TNFα, CCL1, MCP1, and IL1(3 follows the same trend as Wnt5a after DHA (FIG. 23J). MMP13, MMP2 and MMP9 expression is enhanced when Wnt5a-ROR2 is activated (Yamagata et al., 2012). The 3 mRNAs showed the same trend of expression as Wnt5a (FIG. 24J), indicating the activation of ROR2 by Wnt5a. Other genes, such as E-selectin and ICAM-1, that are involved in inflammatory signaling and are known to be activated by Wnt5a (Kim et al., 2010), were found to follow the same pattern of Wnt5a expression. These results indicate that the effect of Wnt5a on those genes is restricted specifically to the penumbra, not the contralateral side. As Wnt5a is available in both hemispheres, stress is required for Wnt5a to act as an inflammatory mediator. These results altogether point to Wnt5a as a non-conventional inflammation mediator and DHA/NPD1 signaling as a regulatory mechanism that specifically switches off Wnt5a-triggered gene expression and Wnt5a extracellular availability.

Discussion

Wnt5a fosters neuronal survival by negatively regulating the cell cycle (Zhou et al., 2017), protects against neurodegeneration through glucose metabolism promotion (Cisternas et al., 2016) and displays high abundance that correlate with the aggressiveness of cancer (Binda et al., 2017). We found that several forms of Wnt5a are released by human RPE cells and that deglycosylation allows visualization of Wnt5a. We are currently validating whether glycosylation is a cellular regulatory mechanism of Wnt5a extracellular availability. In addition, the context in which Wnt5a targets a cell also determines its activity. By characterizing the vesicles containing FZD5/Wnt5a, FZD5/clathrin or Wnt5a/clathrin, we defined that the variation does not always follow the same pattern for the 3 types of vesicles (FIGS. 21D and E), indicating that other receptors are involved in the inflammatory signaling of hpRPE cells. Wnt5a also triggers the internalization of FZD4 (Chen et al., 2003), though whether or not this pathway is conducive to the activation of NFkB is unclear. Moreover, the presence of Wnt5a enhanced apoptosis beyond the levels induced by UOS, resembling TNFα action (FIG. 18D and FIG. 24), whereas the sole presence of Wnt5a did not trigger RPE cell death (FIG. 18D and FIG. 24C). Thus, within cells in a susceptible state, Wnt5a affects their fate since they may succumb to initial insults, such as UOS, adding another layer of regulation to the Wnt ligand function. Insults and exposure to Wnt5a enhances susceptibility to containment of cell integrity and survival.

Wnt ligands are secreted via exosomes (Gross et al., 2012), but Wnt5a was not in the exosomal form in hpRPE cells when we extracted them by ultracentrifugation (FIG. 20F) and by differential solubility. Only by precipitation of 100,000 rpm supernatant did we rescue the sWnt5a (FIG. 20F). Therefore, in RPE cells under UOS, our data show that even Wnt5a is trafficked via vesicles (FIG. 22H), and its release is produced mainly in an exosome-free manner (FIG. 20F). Wnt5a signal was found alone and together with FZD5, signifying that at least 2 distinctive events occur in RPE cells (FIGS. 21A, D, G and H). Wnt5a signal increases with the addition of recombinant Wnt5a (FIG. 21F and FIG. 23B), reflecting the ability of Wnt5a to enhance not only its own expression but also FZD4, 2, and 5 receptor endocytosis (Chen et al., 2003; Kurayoshi et al., 2007; Shojima et al., 2015). Vesicular Wnt5a was detected in RPE cells (FIG. 21 and FIG. 22) as was secretion of mature Wnt5a in vesicles, and Golgi supported maturation of the protein (Kurayoshi et al., 2007). Therefore, different size vesicles carrying Wnt5a detected in RPE cells (FIG. 21F) can harness maturation, degradation and sorting of Wnt5a through a dynamic, steady state with intracellular Wnt5a remaining constant (FIG. 20A-D) while extracellular release is controlled; transcription is variable depending on UOS (FIG. 18D and FIG. 19C; FIG. 25 and FIG. 25). NPD1 decreases soluble Wnt5a during UOS (FIG. 20A-D and FIG. 22D) while CME-inhibitor, Pitstop2, interrupted Wnt5a endocytosis in resting cells and in NPD1 treated cells, ensuing in an increase in sWnt5a under these conditions (FIG. 22D) and pointing to the role of NPD1 in the fate of secreted Wnt5a.

As a macrophage activator in the non-sterile innate response (Pereira et al., 2008), Wnt5a also mediates non-sterile inflammatory responses in non-immune cells (Zhao et al., 2014). Our current results indicate that non-immune cells evoke the inflammatory response in sterile conditions. The signaling pathways involved in RPE cell damage and in stroke display similarities in the stroke penumbra, Wnt5a synthesis increase (FIGS. 23F and I) in the ipsilateral side (FIG. 23F), Wnt5a protein elevation in both sides (FIG. 23I) and presence in the blood plasma after stroke, providing a potential stroke biomarker candidate. Systemic Wnt5a can be involved in the recruiting and stimulation of innate immune cells since Wnt5a activates microglia, dendritic cells and macrophages (Shimizu et al., 2016), enhancing other pathways such as non-canonical Wnt and TLR-triggered signaling. DHA treatment brought down bloodstream Wnt5a protein after 24 hours of stroke onset (FIG. 23G). This was an early event considering that at 3-7 days after stroke the levels of Wnt5a in blood decreased. Box5 administration did not affect Wnt5a abundance as DHA did, indicating that Box5 and DHA act differently. Without wishing to be bound by theory, DHA conversion into NPD1 activates cRel expression (Calandria et al., 2015), which in turn halts Wnt5a transcription, release and autocrine and paracrine binding to FZD5, while Box5 impedes the latter. Our data show Box5 and DHA protection at the neurological/behavioral level as well as by reduction of stroke damage (FIG. 23C-E); however, DHA seems to produce a sustained improvement. The source of circulating Wnt5a is unknown and currently under investigation. There are at least 2 sources of secreted Wnt5a: blood cells such as monocytes (Sessa et al., 2016) or endothelial cells that produce the Wnt ligand in certain conditions, inducing permeabilization and angiogenesis (Korn et al., 2014; Skaria et al., 2017). The elevation in Wnt5a found in the contralateral hemisphere (FIG. 23F), can be explained by damage or permeabilized brain blood barrier that allows the entrance of the circulating Wnt ligand. Alternatively, without increasing its transcription, the hike in the contralateral Wnt5a protein may be explained by glutamate excitotoxicity in the contralateral area at a lesser degree than in the ipsilesional side (Li et al., 2012). Finally, gene expression linked to Wnt5a, UOS and inflammation via NFkB/p65 or Wnt signaling markedly increased in ipsilateral but not in the contralateral side, and such an increase was counteracted by DHA as indicated by the gene expression profile (FIG. 23J).

Transcellular inflammation signaling is not well understood. Without wishing to be bound by theory, non-immune cells under UOS conditions can transfer inflammatory signals that may only affect susceptible cells and lead to their damage. NPD1 interferes with the Wnt5a feedback loop at 2 strategic signaling points, promoting cell survival in bystander cells. The ischemic stroke model provided an in vivo test of our observations at the RPE cell level. We have previously found that this lipid mediator synthesis correlated with cRel, a member of NFkB/p65, activity in RPE cells and in post-stroke penumbra (Calandria et al., 2015). We demonstrate that cRel binds to at least 2 regions in the Wnt5a promoter with high affinity (FIG. 19D-F and Table S4). cRel overexpression suppresses the Wnt5a transcription in response to UOS probably displacing those NFkB dimers containing p65 (FIG. 19A-C). Post-stroke, we observed a similar trend seen in the RPE: Wnt5a transcription was elevated at 1 and 3 days after stroke only in penumbra at the ipsilateral hemisphere while the expression in the contralateral side was not affected (FIG. 23F). These results indicate that only susceptible cells affected directly by ischemia reperfusion promote the Wnt5a positive feedback loop at the transcriptional level. DHA enhances NPD1 synthesis and induces translocation of cRel in neurons (Calandria et al., 2015), which prevents p65-driven activation of Wnt5a transcription. Stressors like NMDA glutamate receptor activation trigger transcription-independent Wnt5a translation (Li et al., 2012), which means that Wnt5a transcription and translation may be uncoupled events and may explain some of our observations (FIGS. 23F and I). Therefore, without wishing to be bound by theory, translation alone is linked to secretion and sequestration from the extracellular space via CME in the RPE (FIG. 22H) and may be plausible in ischemic stroke as well.

These findings uncovered a new participant in the transfer of inflammatory signals occurring in retina and brain under UOS and how endogenous neuroprotection mediators derived from DHA may halt damage to enhance cell survival. The understanding of these new neuroprotective cellular and molecular mechanisms will allow the exploration of therapeutic avenues to target onset and early progression of brain and retina damage that include neurodegenerative diseases.

Experimental Model and Subject Details

Transient Middle Cerebral Artery Occlusion (MCAo)

Animals were housed and treated in compliance of LSU Health Sciences Center Institutional Animal Care and Use Committee (IACUC) protocols. The right MCA was occluded for 2 h by intraluminal filament, as we described previously (Belayev et al., 2011). Briefly, the right common carotid artery (CCA) and external carotid artery (ECA) were exposed through midline neck incision, and then completely isolated from the surrounding nerves. The occipital branches of the ECA and pterygopalatine artery were ligated. A 4-cm of 3-0 nylon filament, coated with poly-L-lysine was advanced to the origin of MCA through the proximal ECA via internal carotid artery. The filament was inserted 20 to 22 mm from the bifurcation of the CCA, according to the animal's body weight. The neck incision was then closed and the rats were returned to their cages. After 2 h of MCAo, the rats were re-anesthetized with the same anesthetic combination and the intraluminal filament was gently removed. The animals were allowed to survive for different times, according to the experimental protocol, with free access to water and food.

Cell Culture, Treatments, and Transfection

Primary human RPE cultures were obtained from human eyecups, provided by the National Disease Research Interchange (NDRI), as described previously (Calandria et al., 2012). hRPE cells were grown and maintained in high-glucose MEM (Life Technologies Corporation) supplemented with 10% FBS (Tissue Culture Biologicals, Inc.), 5% NCS, non-essential amino acids, Penicillin-Streptomycin (100 U/mL), human fibroblast growth factor (FGF) 10 ng/mL and incubated at 37° C. with a constant supply of 5% CO2. ARPE-19 cells, were plated and grown in DMEM/F-12 containing 10% FBS and 1× penicillin/streptomycin at 37° C., 5% CO2, 99% relative humidity for 24 h. Silenced 15-LOX-1 cells, described in detail elsewhere (Calandria et al., 2009), are derived from ARPE-19 by stably silencing 15-LOX-1. 15-LOX-1 deficient cells were maintained in the same medium as ARPE-19 with the addition of 500 μg/mL Geneticin (Life Technologies Corporation). Transfection was performed in all cases using Lipofectamine 2000 (Life Technologies Corporation) following the manufacturer's recommendations. Transfection efficiency was assessed using an expression plasmid-carrying GFP ORF or by including a negative control siRNA conjugated with Alexa Fluor® 488 or 546 (QIAGEN). siRNA efficiency was assessed using SYBR green-based real-time PCR using the primers of Table S4 and the percentage of silencing achieved (FIG. 24). Experiments with transfection efficiency that yielded less than 80% were discarded. For siRNA, we used 6 μl of Lipofectamine 2000 per 50 pmol of siRNA per mL of incubation medium, and, for plasmids, we used 6 μl of Lipofectamine 2000 per 2 μg of plasmid per mL of incubation medium. Cells were incubated for 5 hours at 37° C., 5% CO2 and 99% relative humidity. After the medium was changed, the cells were left to recover for 24 hours before starting treatments. When siRNA/plasmid co-transfection was required, siRNA and plasmid-carrying transfection mixes were prepared separately and added one after the other in the same well. Oxidative stress treatments were performed using 600 (ARPE-19) or 1600 (hRPE) μM H2O2, along with 10 ng/mL TNFα, for the required time indicated in each experiment (these concentrations were previously tested to achieve over 50% apoptosis on ARPE-19 and hpRPE cells). The treatment we used employed the low serum (0.5% serum) without hFGF. Treatment to induce uncompensated oxidative stress (UOS) was performed after 8 hours of low-serum medium incubation unless specifically noted. Wnt5a, Wnt3a or NPD1 was added immediately before induction of UOS. In the secretion experiments, Pitstop 2 was added to the medium 2 hours before harvesting.

Immunocytochemistry and Hoechst Staining

Immunostaining was performed in 8-well slide chambers and the Hoechst staining in 24 well plates. To carryout immunocytochemistry, a previously described protocol was followed (Calandria et al., 2012). Briefly, cells were fixed using 4% paraformaldehyde in PBS 1× for 20 min at room temperature or overnight at 4° C. After three washes with PBS, cells were permeabilized using 0.1% Triton™ X-100 for five minutes and blocked using 10% normal serum and 1% BSA for one hour. Primary antibody incubation took place overnight at 4° C. and secondary antibody coupled to Alexa Fluor® 488 and 594 were used to detect Wnt5a and FZD5 respectively. Hoechst staining was performed using same protocol of fixation, but permeabilization was done using methanol for 20 min at room temperature. Immediately after 20 ng/mL Hoechst 33342 (Life Technologies) in PBS 1× was added. Images for Hoechst staining were obtained using Nikon Ti-U inverted fluorescence microscopes with NIS-Elements BR 3.00 software (NIKON Inc, Melville, N.Y., USA) and ICC images from Olympus FV1200 confocal with Fluoview software FV10-ASW Version 04.02.02.09 (Olympus Corp Center Valley, Pa., USA). The analysis was performed using ImageJ 1.48 (National Institutes of Health). Dying cells were identified by size (0 to 50 pixels), intensity threshold and circularity. The ratio of hyperpyknotic cells over the total was calculated for nine randomly chosen fields per sample obtained from three independent wells per experiment. Each experiment was repeated at least three times to confirm the findings. In the experiments involving early stages of apoptosis, we assessed the total number of cells per field and counted them to ensure unbiased observations. Colocalization of Wnt5a and FZD5 was performed using BioImageXD (Kankaanpaa et al., 2012) and IMARIS 9.3 (Bitplane, Oxford instruments, Belfast, UK) on z-stack images obtained at 20× magnification in an Olympus FluoView1200 confocal laser-scanning microscope. Settings were adjusted in the conditions that showed higher intensity and were used throughout the remaining samples. Pearson's colocalization coefficient (PCC) was obtained using FV1200 analysis software for every field in each experiment, and the mean and SEM are depicted in Figures S1-S5. Validation of the FZD5 primary antibody used in the study is depicted in Figure S2. Colocalized objects and plots of pixels vs intensity were obtained using ImageJ (Schneider et al., 2012). IMARIS software analysis was performed building a colocalization channel using the Costes thresholding, and the colocalized elements were analyzed using the spots function using the settings different spots sites, background subtraction and local contrast. The output obtained consisted on a collection of sizes (one per element in each picture) or the mean of the intensity in each spot. The data was plotted as boxplot and analyzed using BioVinCi 1.1.5 (Bioturing, San Diego, Calif., USA).

Protein Precipitation and Western Blot

When secretion of Wnt5a was assessed, 1 mL of medium was collected, centrifuged at 13,000 rpm/5 min at 4° C. to remove cell debris and precipitated using methanol/chloroform (Wessel and Flügge, 1984). The pellet was re-suspended and denatured in 100 μl of 2× Laemmli sample buffer (Bio-Rad Laboratories) at 95° C. for 5 min. MCAo brain tissue and cell samples were homogenized using RIPA (Thermo Fisher Scientific) buffer supplemented with protease and phosphatase inhibitor cocktails (Sigma). The amount of 30 μg of total protein was loaded in NuPAGE® Novex® 4-12% Bis-Tris precast gels (Life Technologies Corporation) or Mini-PROTEAN TGX Stain free gels 4-15% and ran at constant 120V for 1 hour and 20 min or 250 V for 40 min respectively. Proteins were transferred to μm Nitrocellulose or LF-PVDF membrane using Bio-Rad Trans-Blot® Turbo™ System (Bio-Rad Laboratories). ECL™ Plex Fluorescent Rainbow Markers (GE Healthcare) were used as the ladder for protein's molecular weight. Membranes were blocked using 5% non-fat dry milk (Bio-Rad Laboratories) in TBS with 1% Tween® 20 (TBST-10X) for 1 hour and incubated overnight with primary antibodies. Anti-mouse or anti-rabbit secondary antibodies conjugated with Cy3 or Cy5 were used to visualize the protein of interest. Immunoblots were documented using LAS 4000 imaging system (GE Healthcare Life Sciences) or ChemiDoc MP Imaging System (BioRad Laboratories). A time course exposition was produced in each case to prevent quantification of saturated images. Densitometry data was obtained using ImageQuant™ TL software and XRS Blot Chemi Software (BioRad Laboratories).

SYBR Green-Based Real-Time PCR

Brain samples were homogenized on ice by Dounce homogenizer and total RNA was extracted by TRIzol Reagent (Life Technologies Corporation). Cell samples, total RNA was extracted by RNeasy Mini Kit (QIAGEN) following manufacturer's protocol. The purity and concentration of RNA were determined by NanoDrop ND-1000 Spectrophotometer (Thermo Fisher Scientific). cDNA first strand was obtained from one microgram of total RNA using iScript™ Reverse Transcription Supermix (Bio-Rad Laboratories, CA, USA). The resulting cDNA was used as template SYBR-green or Eva-green based real-time PCR quantification using SsoAdvance Universal Supermix (Bio-Rad Laboratories). Data was collected and analyzed using CFX Manager 3.0 software, the ΔΔCt method. Melting curve was produced for every run to assure a unique amplified product per primer set. Primers are depicted in Table S4.

Luciferase Assay

For activation of canonical NF-κB, cells were co-transfected with plasmid p65/p50 promoter consensus sequence Cignal NF-κB Reporter Kit, (QIAGEN) along with both positive (constitutively-active promoter) or negative controls and GFP by using Lipofectamine 2000 (Life Technologies) following company protocols. β-catenin activity was measured using TOP Flash/FOP Flash constructs obtained from Addgene (Cambridge, Mass., USA) (Veeman et al., 2003). Except TOP/FOP flash, the Luciferase activity was standardized using a construct expressing GFP constitutively under CMV virus promoter. To assess luciferase activity, cell lysates were obtained using Passive Lysis Buffer (Promega) and mixed with a luciferase assay reagent (Promega). Chemiluminescence produced by luciferase and fluorescence from GFP was detected using Appliscan 2.3. Data were analyzed using SkanIt 2.3 (Thermo Fisher Scientific).

Detection of DNA Binding Motifs and Methylation

To analyze the consensus binding sequences of the Wnt5a promoter A (Katula, et al., 2012), two searching engines were used: TRED (http://rulai.cshl.edu/TRED), which uses the JASPAR database, and TFBind (http://tfbind.hgc.jp/) that uses TRASFAC database (Jiang et al., 2007; Tsunoda and Takagi, 1999). Table S1 contains scores and positions obtained for the 7 regions identified that cREL potentially binds (FIG. 19). Analysis of the methylation was performed using MethPrimer (http://www.urogene.org/methprimer2/) (Li and Dahiya, 2002).

ChIP Assay

The chromatin immunoprecipitation assay was performed using SimpleChIP® Plus Enzymatic Chromatin IP Kit (Cell Signaling Technology, Boston, Mass., USA) following the manufacturer's recommendations including ChIP validated cREL antibody. Positive control Histone H3 antibody and normal serum was provided by the kit, as well as control primers for human RPL30 Exon 3. Primers A1 to 4 were designed using Primer-Blast at NCBI platform (Ye et al., 2012) and are depicted in Table S5. The immunoprecipitated samples real-time PCR values were standardized using 10% of the input chromatin preparation using primers A1, A2, and A3 pooled values and RPL30 Exon 3 values.

Behavioral Tests

Behavioral tests were conducted before, during MCAo (at 60 min), and then at 24 h, 48 h, 72 h, or 7 days after MCAo by an investigator who was blinded to the experimental groups. The battery consisted of two tests, (1) postural reflex to examine the upper body posture when the rat was suspended by tail, and (2) forelimb placing test to assess the forelimb placing responses to visual, tactile and proprioceptive stimuli (Belayev et al., 2011). Neurologic function was graded on a scale of 0 to 12 (normal=0, maximal deficits=12), as we described previously (Belayev et al., 2011). The severity of stroke injury was assessed by behavioral examination of each rat at 60 min after onset of MCAo. Rats that did not demonstrate high-grade contralateral deficit (score, 10-11) were excluded from further study. Two animals were excluded for this reason.

Treatment Groups

Docosahexaenoic acid (DHA; 5 mg/kg, Cayman, Ann Arbor, Mich., USA) or vehicle (0.9% saline) was administered intravenously into the femoral vein at a constant rate over 3 min using an infusion pump at 3 h after onset of MCAo. For western-blot study rats were sacrificed on days 1, 3, or 7; for real-time PCR study rats were sacrificed on days 1, 2, or 3.

Quantification and Statistical Analysis

Data is presented as histograms with mean values±SD or Boxplot, which depict median quartiles 1, 3 and maximum and minimum observations. Repeated measures analysis of variance (ANOVA) followed by Bonferroni procedures to correct for multiple comparisons was used for intergroup comparisons. Multiple comparisons was performed using Tukey's Honest Significant Difference. Plotting and statistical analysis was performed using Excel 2016 or BioVinCi 1.1.5 (developed by BioTuring Inc., San Diego, Calif., USA, www.bioturing.com). Differences at P<0.05 were considered statistically significant.

Data and Software Availability

NIS-Elements BR 3.00, FV10-ASW Version 04.02.02.09, ImageQuant™ TL, LAS 4000 imaging system, LAS 4000 imaging system, CFX Manager 3.0, MiniTab 18, SkanIt 2.3, IMARIS 9.3.1, BioVinci 1.1.5, XRS Blot Chemi Software and Excel 2016, are licensed software and the licenses were own by NG Bazan Lab. ImageJ 1.48, BioImageXD, TRED, and TFBind are free resources available online at the websites mentioned in the key resources table

Supplemental Table Titles

Table S1. TRED and TF bind analysis on the Promoter sequence (Katula et al. 2012), Related to FIG. 20, D-G. Table S1 discloses SEQ ID NOS 43-57, 43, 58, 48, 59, 50-51, 60-66, 56, 67-68, 48, 69, 50, 70-71, 61-62, 72, 65 and 73-75, respectively, in order of appearance.

TABLE S1
[TRED and TF bind analysis on the Promoter sequence
(Katula et al., 2012)], Related to FIGS. 3D-3G.

| | | |
|---|---|---|
| Position/Sequence | TRED Score | TFBind Score |

cREL

| | | | |
|---|---|---|---|
| Region 1 | [192 . . . 201] TAGAAATTCC | 3.92 | |
| | [214 . . . 223] CCGGTTTTGC | 2.21 | |
| | [215 . . . 224] CGGTTTTGCC | 3.3 | |
| | 193 (+) SGGRNWTTCC TAGAAATTCC | | 0.819522 |
| | 194 (−) SGGRMWTTCC AGAAATTCCG | | 0.844549 |
| Region 2 | [357 . . . 366] GGGACTTTGC | 5.08 | |
| | 358 (+) SGGRNWTTCC GGGACTTTGC | | 0.854004 |
| Region 3 | [1445 . . . 1454] GCGACTTTCA | 4.12 | |
| Region 4 | [1550 . . . 1559] CGGCATCTCC | 3.3 | |
| | 1565 (−) SGGRNWTTCC GAAAAAGCCA | | 0.850945 |
| Region 5 | [1945 . . . 1954] CCTAATTACC | 1.99 | |
| | 1939 (−) SGGRNWTTCC GGAAAGCCCT | | 0.887097 |
| Region 6 | [2103 . . . 2112] GGGCGCATCC | 2.6 | |
| Region 7 | [2284 . . . 2293] GGCGACTTCC | 3.71 | |
| | 2285 (+) SGGRNWTTCC GGCGACTTCC | | 0.814516 |

p65

| | | | |
|---|---|---|---|
| Region 1 | [192 . . . 201] TAGAAATTCC | 4.17 | |
| | 194 (−) GGGRATTTCC AGAAATTCCG | | 0.868024 |
| Region 2 | [357 . . . 366] GGGACTTTGC | 6.19 | |
| | 358 (+) GGGRATTTCC GGGACTTTGC | | 0.861557 |
| Region 3 | [1445 . . . 1454] GCGACTTTCA | 1.95 | |
| Region 4 | [1550 . . . 1559] CGGCATCTCC | 2.56 | |
| | 1551 (+) GGGRATTTCC CGGCATCTCC | | 0.769102 |
| | 1552 (−) GGGAMTTYCC GGCATCTCCC | | 0.803246 |
| Region 5 | [1937 . . . 1946] TGGAAAGCCC | 2.14 | |
| | 1938 (+) GGGRATTTCC TGGAAAGCCC | | 0.782754 |
| | 1939 (−) GGGRATTTCC GGAAAGCCCT | | 0.86491 |
| Region 6 | [2104 . . . 2113] GGCGCATCCC | 1.79 | |
| | 2105 (+) GGGRATTTCC GGCGCATCCC | | 0.765749 |
| Region 7 | [2284 . . . 2293] GGCGACTTCC | 2.72 | |
| | 2285 (+) GGGRATTTCC GGCGACTTCC | | 0.771018 |

NFkB/p50

| | | | |
|---|---|---|---|
| Region 1 | 193 (−) NGGGACTTTCCA TAGAAATTCCGG | | 0.760042 |
| Region 2 | [357 . . . 366] GGGACTTTGC | 5.92 | |
| | 358 (+) GGGGATYCCC GGGACTTTGC | | 0.750555 |
| Region 3 | [1445 . . . 1454] GCGACTTTCA | 0.86 | |
| Region 4 | [1551 . . . 1560] GGCATCTCCC | 1.74 | |
| | 1551 (−) GGGGATYCCC CGGCATCTCC | | 0.790315 |
| | 1552 (−) GGGAMTTYCC GGCATCTCCC | | 0.803246 |
| Region 5 | [1937 . . . 1946] TGGAAAGCCC | 2.08 | |
| | 1939 (−) GGGGATYCCC GGAAAGCCCT | | 0.79498 |
| Region 6 | [2104 . . . 2113] GGCGCATCCC | 3.78 | |
| | 2105 (−) GGGGATYCCC GGCGCATCCC | | 0.75522 |
| Region 7 | [2285 . . . 2294] GCGACTTCCT | 2.95 | |
| | 2285 (+) GGGGATYCCC GGCGACTTCC | | 0.754331 |

Table S2. CpG islands detected by MethPrimer (Li and Dahiya, 2002). Criteria: Island size >100, GC Percent >50.0, Obs/Exp >0.6): 5 CpG island(s) were found in the sequence, Related to FIG. 20, E-G.

TABLE S2
CpG islands detected by MethPrimer (Li and Dahiya,
2002). Criteria: Island size >100, GC Percent >50.0,
Obs/Exp >0.6): 5 CpG island(s) were found in the sequence],
Related to FIGURES 3E, 3F, and 3G.

| | Size | (Start-End) |
|---|---|---|
| Island 1 | 175 bp | (137-311) |
| Island 2 | 173 bp | (483-655) |
| Island 3 | 144 bp | (664-807) |
| Island 4 | 793 bp | (948-1740) |
| Island 5 | 338 bp | (1929-2266) |

Table 3. DNA constructs and siRNAs, Related to FIG. 18, G-J, and FIG. 19, B-C. Table S3 discloses “7 TCF/LEF binding sites: AGATCAAAGGgggta” as SEQ ID NO: 2.

TABLE S3
DNA constructs and siRNAs, Related to FIGURES 1G-1J and 2B-2C.

| | | | Reference/catalog |
|---|---|---|---|
| | | | number and |
| Constructs | Type | Gene/protein | company |
| Wild Type | REL (untagged)- | REL (NM_002908) | Origene True ORF |
| cREL | UNIQUE VARIANT | | Cat #SC126639 |
| expression | 1 of Human v-rel | | |
| vector | reticuloendotheliosis | | |
| | viral oncogene | | |
| | homolog (avian) (REL) | | |
| NFkB | 3 tandem copies of p65 | | Qiagen, Cignal |
| reporter | binding sequence driving | | NFκB Reporter |
| vector | the expression of | | (luc) Kit: |
| | luciferase. | | Cat #CCS-013L |
| Human | Open Reading frame | | Origene True ORF |
| Wnt5a | | | Cat #SC126838 |
| variant 1 | | | |
| Human | Open Reading frame | | Origene True ORF |
| FZD₅ | | | Cat #SC117952 |
| Human | Open reading Frame | | Origene True ORF |
| ROR2 | | | Cat #SC117279 |
| TOP | Super8XTOPflash | 7 TCF/LEF | Addgene repository |
| Flash | construct M50, Beta- | binding sites: | Plasmid #12456. |
| | catenin reporter. | AGATCAAAGGgggta | Zebrafish prickle, a |
| | TCF/LEF sites | with TCF/LEF | modulator of |
| | upstream of a | binding site | noncanonical |
| | luciferase reporter. | in CAP letters, and a | Wnt/Fz signaling, |
| | | spacer in lower case, | regulates gastrulation |
| | | separating each copy | movements. |
| | | of the TCF/LEF site). | (Veeman et al, 2003) |
| FOP | M51 Super | 6 mutated TCF/LEF | Addgene repository |
| flash | 8 × TOPFlash | binding sites that were | Plasmid #12457. |
| | (TOPFlash mutant) | cloned into the pGL3 | (Veeman et al, 2003) |
| | | vector (Promega) | |
| FZD5 | Human FZD5 21-mer | Human FZD5 | Silencer select |
| siRNA | siRNA duplexes | (NM_003468) | Validated Ambion, |
| | | | Life Technologies- |
| | | | Thermo |
| | | | Cat #4390824. |
| | | | ID: s15416 |
| ROR2 | Human ROR2 21-mer | Human ROR2 | Silencer select |
| siRNA | siRNA duplexes | (NM_004560) | Ambion, Life |
| | | | Technologies- |
| | | | Thermo |
| | | | Cat #4390824. |
| | | | ID: s9758 |
| Negative | Non-specific binding | Proprietary | Allstars Qiagen |
| control | siRNA sequence | | Cat #1027292 |
| siRNA | | | |
| Alexa | | | |
| Fluor 488 | | | |
| conjugated | | | |

Table S4. Primers information, Related to FIG. 18, C, E, F and FIGS. 23, F and J. Table S4. disclose SEQ ID NOS 3-10, 82, 76, 13-34 and 77-78, respectively, in order of appearance.

TABLE S4
Primers information, Related to FIGS. 1C, 1E, 1F, 6F and 6J.

| Target | Sequence | Source |
|---|---|---|
| Rat Wnt5a | Forward primer | RealTimePrimers.com |
| | 5′-TTACCCAAACCGGACTGTTA-3′ | |
| | Reverse primer | |
| | 5′-AGCCTTTTCGGTTCATCTCT-3′ | |
| Human Wnt5a | Forward primer | Campioni et al., 2008. |
| | 5′-CAAAGCAACTCCTGGGCTTA-3′ | |
| | Reverse primer | |
| | 5′-CCTGCTCCTGACCGTCC-3′ | |
| Rat Chemokine | Forward primer | RealTimePrimers.com |
| C-X-X motif | 5′-GCGGAGAGATGAGAGTCTGG-3′ | |
| ligand 1 (Cxcl1) | Reverse primer | |
| NM_030845 | 5′-TCCAAGGGAAGCTTCAACAC-3′ | |
| Rat ACTB | Forward primer | RealTimePrimers.com |
| | 5′-CACACTGTGCCCATCTATGA-3′ | |
| | Reverse primer | |
| | 5′-CCGATAGTGATGACCTGACC-3′ | |
| Rat TNFa | Forward primer | RealTimePrimers.com |
| | 5′-AACTCGAGACAAGCCCGTAG-3′ | |
| | Reverse primer | |
| | 5′-GTACCACCAGTTGGTTGTCTTTGA-3′ | |
| Rat IL6 | Forward primer | RealTimePrimers.com |
| | 5′-CTTCCTACCCCAACTTCCAA-3′ | |
| | Reverse primer | |
| | 5′-ACCACAGTGAGGAATGTCCA-3′ | |
| Rat B2m | Forward primer | RealTimePrimers.com |
| | 5′-TGCTACGTGTCTCAGTTCCA-3′ | |
| | Reverse primer | |
| | 5′-GCTCCTTCAGAGTGACGTGT-3′ | |
| Rat MMP13 | Forward primer | RealTimePrimers.com |
| | 5′-CCTCTTCTTCTCAGGGAACC-3′ | |
| | Reverse primer | |
| | 5′-GGAATTTGTTGGCATGACTC-3′ | |
| Rat MMP9 | Forward primer | RealTimePrimers.com |
| | 5′-ACTTCTGGCGTGTGAGTTTC-3′ | |
| | Reverse primer | |
| | 5′-TGTATCCGGCAAACTAGCTC-3′ | |
| Rat MMP2 | Forward primer | RealTimePrimers.com |
| | 5′-CTTCAGGTTCTCCAGCATGA-3′ | |
| | Reverse primer | |
| | 5′-CCGTAAGGGAGACACCAGAT-3′ | |
| Rat IL-1β | Forward primer | Nakazawa et al., 2001. |
| | 5′-TCAGGAAGGCAGTGTCACTCATTG-3′ | |
| | Reverse primer | |
| | 5′-ACACACTAGCAGGTCGTCATCATC-3′ | |
| Rat ICAM1 | Forward primer | Ammirante et al., 2010. |
| | 5′-CTGTCAAACGGGAGATGAATGGT-3′ | |
| | Reverse primer | |
| | 5′-TCTGGCGGTAATAGGTGTAAATGG-3′ | |
| Rat MCP1 | Forward primer | Nakazawa et al., 2006. |
| | 5′-ATGCAGGTCTCTGTCACGCTTCTG-3′ | |
| | Reverse primer | |
| | 5′-GACACCTGCTGCTGGTGATTCTCTT-3′ | |
| Rat E-Selectin | Forward primer | Hannawa et al., 2005. |
| | 5′-TGCGATGCTGCCTACTTGTG-3′ | |
| | Reverse primer | |
| | 5′-AGAGAGTGCCACTACCAAGGGA-3′ | |
| Rat Ywhaz | Forward primer | Gubern et al., 2009. |
| | 5′-GATGAAGCCATTGCTGAACTTG-3′ | |
| | Reverse primer | |
| | 5′-GTCTCCTTGGGTATCCGATGTC-3′ | |
| Rat Sdha | Forward primer | Gubern et al., 2009. |
| | 5′-TCCTTCCCACTGTGCATTACAA-3′ | |
| | Reverse primer | |
| | 5′-CGTACAGACCAGGCACAATCTG-3′ | |
| Human cREL | Forward primer | Calandria et al., 2015 |
| | 5′-CAGGAGGAAGAGCAGTCGTC-3′ | |
| | Reverse primer | |
| | 5′-GCAGGAATCAATCCATTCAA-3′ | |

Table S5. ChIP assay primers for SYBR green based real-time PCR, Related to FIG. 20, E-G. Table S5 discloses the “Forward” sequences as SEQ ID NOS 35-38 and the “Reverse” sequences as SEQ ID NOS 39-42, all respectively, in order of appearance.

TABLE S5
ChIP assay primers for SYBR green based real-time PCR,
Related to FIGS. 3E-3G.

| | | Sequence | |
|---|---|---|---|
| Promoter | Primers | Forward | Reverse |
| Wnt5a | A1 | 5′-GCATCCCACTACCCAAGTCC-3′ | 5′-GCTGCCTTGACATGGAACCTCA-3′ |
| Promoter A | A2 | 5′-CAGCAATAAGTTCCGGGGCG-3′ | 5′-GCTTTGGGGCCACAGAACAATC-3′ |
| | A3 | 5′-GCCTCTCCGTGGAACAGTTGC-3′ | 5′-GATGCGCCCAGGAATGG-3′ |
| | A4 | 5′-CGCCAGTGCCCGCTTCAG-3′ | 5′-CAGCCGAGGAATCCGAGC-3′ |

### REFERENCES CITED IN THIS EXAMPLE

1. Ackers, I., Szymanski, C., Duckett, K. J., McCall, K., and Malgor, R. (2015). Wnt5a Signaling in Atherosclerosis, its Effect on OxLDL Uptake and Foam Cell Differentiation. The FASEB Journal 29, 609.4.
2. Bazan, N. G. (2006). Cell survival matters: docosahexaenoic acid signaling, neuroprotection and photoreceptors. Trends Neurosci. 29, 263-271.
3. Bazan, N. G. (2007). Homeostatic regulation of photoreceptor cell integrity: significance of the potent mediator neuroprotectin D1 biosynthesized from docosahexaenoic acid: the Proctor Lecture. Invest. Ophthalmol. Vis. Sci. 48, 4866-4881; biography 4864-4865.
4. Belayev, L., Mukherjee, P. K., Balaszczuk, V., Calandria, J. M., Obenaus, A., Khoutorova, L., Hong, S.-H., and Bazan, N. G. (2017). Neuroprotectin D1 upregulates Iduna expression and provides protection in cellular uncompensated oxidative stress and in experimental ischemic stroke. Cell Death Differ. 24, 1091-1099.
5. Binda, E., Visioli, A., Giani, F., Trivieri, N., Palumbo, O., Restelli, S., Dezi, F., Mazza, T., Fusilli, C., Legnani, F., et al. (2017). Wnt5a Drives an Invasive Phenotype in Human Glioblastoma Stem-like Cells. Cancer Res. 77, 996-1007.
6. Buga, A.-M., Sascau, M., Pisoschi, C., Herndon, J. G., Kessler, C., and Popa-Wagner, A. (2008). The genomic response of the ipsilateral and contralateral cortex to stroke in aged rats. J Cell Mol Med 12, 2731-2753.
7. Calandria, J. M., Marcheselli, V. L., Mukherjee, P. K., Uddin, J., Winkler, J. W., Petasis, N. A., and Bazan, N. G. (2009). Selective Survival Rescue in 15-Lipoxygenase-1-deficient Retinal Pigment Epithelial Cells by the Novel Docosahexaenoic Acid-derived Mediator, Neuroprotectin D1. J Biol Chem 284, 17877-17882.
8. Calandria, J. M., Mukherjee, P. K., de Rivero Vaccari, J. C., Zhu, M., Petasis, N. A., and Bazan, N. G. (2012). Ataxin-1 Poly(Q)-induced Proteotoxic Stress and Apoptosis Are Attenuated in Neural Cells by Docosahexaenoic Acid-derived Neuroprotectin D1. J Biol Chem 287, 23726-23739.
9. Calandria, J. M., Asatryan, A., Balaszczuk, V., Knott, E. J., Jun, B. K., Mukherjee, P. K., Belayev, L., and Bazan, N. G. (2015). NPD1-mediated stereoselective regulation of BIRC3 expression through cREL is decisive for neural cell survival. Cell Death Differ 22, 1363-1377.
10. Chen, W., ten Berge, D., Brown, J., Ahn, S., Hu, L. A., Miller, W. E., Caron, M. G., Barak, L. S., Nusse, R., and Lefkowitz, R. J. (2003). Disheveled 2 recruits beta-arrestin 2 to mediate Wnt5A-stimulated endocytosis of Frizzled 4. Science 301, 1391-1394.
11. Cisternas, P., Salazar, P., Silva-Alvarez, C., Barros, L. F., and Inestrosa, N. C. (2016). Wnt5a Increases the Glycolytic Rate and the Activity of the Pentose Phosphate Pathway in Cortical Neurons. Neural Plast. 2016, 9839348.
12. De, A. (2011). Wnt/Ca2+ signaling pathway: a brief overview. Acta Biochim. Biophys. Sin. (Shanghai) 43, 745-756.
13. Feng, Q., and Gao, N. (2015). Keeping Wnt signalosome in check by vesicular traffic. J. Cell. Physiol. 230, 1170-1180.
14. Gross, J. C., Chaudhary, V., Bartscherer, K., and Boutros, M. (2012). Active Wnt proteins are secreted on exosomes. Nat. Cell Biol. 14, 1036-1045.
15. Jenei, V., Sherwood, V., Howlin, J., Linnskog, R., Säfholm, A., Axelsson, L., and Andersson, T. (2009). A t-butyloxycarbonyl-modified Wnt5a-derived hexapeptide functions as a potent antagonist of Wnt5a-dependent melanoma cell invasion. Proc. Natl. Acad. Sci. U.S.A. 106, 19473-19478.
16. Katula, K. S., Joyner-Powell, N. B., Hsu, C.-C., and Kuk, A. (2012). Differential regulation of the mouse and human Wnt5a alternative promoters A and B. DNA Cell Biol. 31, 1585-1597.
17. Kim, J., Kim, J., Kim, D. W., Ha, Y., Ihm, M. H., Kim, H., Song, K., and Lee, I. (2010). Wnt5a induces endothelial inflammation via beta-catenin-independent signaling. J. Immunol. 185, 1274-1282.
18. Korn, C., Scholz, B., Hu, J., Srivastava, K., Wojtarowicz, J., Arnsperger, T., Adams, R. H., Boutros, M., Augustin, H. G., and Augustin, I. (2014). Endothelial cell-derived non-canonical Wnt ligands control vascular pruning in angiogenesis. Development 141, 1757-1766.
19. Kurayoshi, M., Yamamoto, H., Izumi, S., and Kikuchi, A. (2007). Post-translational palmitoylation and glycosylation of Wnt-5a are necessary for its signalling. Biochem. J. 402, 515-523.
20. Li, Y., Li, B., Wan, X., Zhang, W., Zhong, L., and Tang, S.-J. (2012). NMDA receptor activation stimulates transcription-independent rapid wnt5a protein synthesis via the MAPK signaling pathway. Mol Brain 5, 1.
21. Mikels, A. J., and Nusse, R. (2006). Purified Wnt5a protein activates or inhibits beta-catenin-TCF signaling depending on receptor context. PLoS Biol. 4, el 15.
22. Mukherjee, P. K., Marcheselli, V. L., Serhan, C. N., and Bazan, N. G. (2004). Neuroprotectin D1: a docosahexaenoic acid-derived docosatriene protects human retinal pigment epithelial cells from oxidative stress. Proc. Natl. Acad. Sci. U.S.A. 101, 8491-8496.
23. Mukherjee, P. K., Marcheselli, V. L., Barreiro, S., Hu, J., Bok, D., and Bazan, N. G. (2007). Neurotrophins enhance retinal pigment epithelial cell survival through neuroprotectin D1 signaling. Proc. Natl. Acad. Sci. U.S.A. 104, 13152-13157.
24. Naskar, D., Maiti, G., Chakraborty, A., Roy, A., Chattopadhyay, D., and Sen, M. (2014). Wnt5a-Rac1-NF-κB homeostatic circuitry sustains innate immune functions in macrophages. J. Immunol. 192, 4386-4397.
25. Nusse, R., and Clevers, H. (2017). Wnt/0-Catenin Signaling, Disease, and Emerging Therapeutic Modalities. Cell 169, 985-999.
26. Pereira, C., Schaer, D. J., Bachli, E. B., Kurrer, M. O., and Schoedon, G. (2008). Wnt5A/CaMKII signaling contributes to the inflammatory response of macrophages and is a target for the antiinflammatory action of activated protein C and interleukin-10. Arterioscler. Thromb. Vasc. Biol. 28, 504-510.
27. Rauner, M., Stein, N., Winzer, M., Goettsch, C., Zwerina, J., Schett, G., Distler, J. H. W., Albers, J., Schulze, J., Schinke, T., et al. (2012). WNT5A is induced by inflammatory mediators in bone marrow stromal cells and regulates cytokine and chemokine production. J. Bone Miner. Res. 27, 575-585.
28. Ring, L., Neth, P., Weber, C., Steffens, S., and Faussner, A. (2014). β-Catenin-dependent pathway activation by both promiscuous “canonical” WNT3a-, and specific “noncanonical” WNT4- and WNT5a-FZD receptor combinations with strong differences in LRP5 and LRP6 dependency. Cell. Signal. 26, 260-267.
29. Sato, A., Kayama, H., Shojima, K., Matsumoto, S., Koyama, H., Minami, Y., Nojima, S., Morii, E., Honda, H., Takeda, K., et al. (2015). The Wnt5a-Ror2 axis promotes the signaling circuit between interleukin-12 and interferon-γ in colitis. Sci Rep 5, 10536.
30. Sen, M., Chamorro, M., Reifert, J., Corr, M., and Carson, D. A. (2001). Blockade of Wnt-5A/frizzled 5 signaling inhibits rheumatoid synoviocyte activation. Arthritis Rheum. 44, 772-781.
31. Sessa, R., Yuen, D., Wan, S., Rosner, M., Padmanaban, P., Ge, S., Smith, A., Fletcher, R., Baudhuin-Kessel, A., Yamaguchi, T. P., et al. (2016). Monocyte-derived Wnt5a regulates inflammatory lymphangiogenesis. Cell Res. 26, 262-265.
32. Shimizu, T., Smits, R., and Ikenaka, K. (2016). Microglia-induced activation of non-canonical Wnt signaling aggravates neurodegeneration in demyelinating disorders. Mol. Cell. Biol.
33. Shojima, K., Sato, A., Hanaki, H., Tsujimoto, I., Nakamura, M., Hattori, K., Sato, Y., Dohi, K., Hirata, M., Yamamoto, H., et al. (2015). Wnt5a promotes cancer cell invasion and proliferation by receptor-mediated endocytosis-dependent and -independent mechanisms, respectively. Sci Rep 5, 8042.
34. Skaria, T., Bachli, E., and Schoedon, G. (2017). Wnt5A/Ryk signaling critically affects barrier function in human vascular endothelial cells. Cell Adh Migr 11, 24-38.
35. Vaidya, H., Rumph, C., and Katula, K. S. (2016). Inactivation of the WNT5A Alternative Promoter B Is Associated with DNA Methylation and Histone Modification in Osteosarcoma Cell Lines U2OS and SaOS-2. PLoS ONE 11, e0151392.
36. Varela-Nallar, L., Alfaro, I. E., Serrano, F. G., Parodi, J., and Inestrosa, N. C. (2010). Wingless-type family member 5A (Wnt-5a) stimulates synaptic differentiation and function of glutamatergic synapses. Proc. Natl. Acad. Sci. U.S.A. 107, 21164-21169.
37. Willert, K., and Nusse, R. (2012). Wnt proteins. Cold Spring Harb Perspect Biol 4, a007864.
38. Yamagata, K., Li, X., Ikegaki, S., Oneyama, C., Okada, M., Nishita, M., and Minami, Y. (2012). Dissection of Wnt5a-Ror2 signaling leading to matrix metalloproteinase (MMP-13) expression. J. Biol. Chem. 287, 1588-1599.
39. Zhao, Y., Wang, C.-L., Li, R.-M., Hui, T.-Q., Su, Y.-Y., Yuan, Q., Zhou, X.-D., and Ye, L. (2014). Wnt5a promotes inflammatory responses via nuclear factor κB (NF-κB) and mitogen-activated protein kinase (MAPK) pathways in human dental pulp cells. J. Biol. Chem. 289, 21028-21039.
40. Zhou, L., Chen, D., Huang, X.-M., Long, F., Cai, H., Yao, W.-X., Chen, Z.-C., Liao, Z.-J., Deng, Z.-Z., Tan, S., et al. (2017). Wnt5a Promotes Cortical Neuron Survival by Inhibiting Cell-Cycle Activation. Front Cell Neurosci 11, 281.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

1. A composition comprising a therapeutically effective amount of an FZD5 receptor blocker, a therapeutically effective amount of a long chain fatty acid, and a pharmaceutically acceptable carrier.

2. The composition of claim 1, wherein the long chain fatty acid comprises a docosanoid, an elovanoid, or a combination thereof.

3. The composition of claim 1, wherein the FZD5 receptor blocker comprises a peptide comprising SEQ ID NO: 1 (NH-Met-Asp-Gly-Cys-Glu-Leu-CO2H).

4. The composition of claim 1, wherein the FZD5 receptor blocker interacts with an extracellular domain of the FZD5 receptor.

5. The composition of claim 4, wherein the extracellular domain comprises amino acids 28 to 150 and/or amino acids 28 to 238.

6. The composition of claim 3, wherein the peptide is N-terminally butyloxycarbonyl (Boc) protected.

7. The method of claim 1, wherein the fatty acid derived from DHA, EPA, omega-3, or a combination thereof.

8. The composition of claim 2, wherein the docosanoid comprises DHA, neuroprotectins; lipoxin A4; DHA-derived Resolvins; Maresin 1; 10R, 17R diHDHA and its methyl ester derivatives; 10S, 17S diHDHA and its methyl ester derivatives; or any combination thereof.

9. The composition of claim 8, wherein the neuroprotectin comprises NPD1.

10. The composition of claim 2, wherein the elovanoid comprises mono-hydroxylated elovanoid, a di-hydroxylated elovanoid, an alkynyl mono-hydroxylated elovanoid, and an alkynyl di-hydroxylated elovanoid, or any combination thereof.

11. The composition of claim 1, wherein the composition further comprises a MicroRNA.

12. The composition of claim 11, wherein the microRNA is miRNA-224.

13. (canceled)

14. A method for treating a patient afflicted with a condition characterized by neuronal damage and/or neuronal injury, the method comprising administering to the patient a therapeutically effective amount of an FZD5 receptor blocker/antagonist and a therapeutically effective amount of a fatty acid.

15. The method of claim 14, wherein the FZD5 receptor blocker and the fatty acid are administered simultaneously.

16. The method of claim 14, wherein the FZD5 receptor blocker and the fatty acid are administered as a single-dose pharmaceutical composition/formulation.

17. The method of claim 14, wherein the FZD5 receptor blocker and/or fatty acid are administered enterally or parenterally.

18. The method of claim 17, wherein the enteral administration is oral or rectal.

19. The method of claim 17, wherein the parenteral administration is selected from the group consisting of intravascular administration; subcutaneous injection, subcutaneous deposition intramuscular injection, intraperitoneal injection, transdermal, nasal and inhalational.

20. The method of claim 14, wherein the condition comprises ischemic stroke.

21. The method of claim 14, wherein the neuronal damage or neuronal injury is the result of or exacerbated by uncompensated oxidative stress.

22. The method of claim 14, further comprising the steps of:
obtaining a sample from the patient;
measuring the protein level of Wnt5a protein in the sample and comparing the protein level of Wnt5a protein in the sample to a control sample;
wherein the patient is treated if the protein level of Wnt5a protein is changed compared to the control.

23. The method of claim 22, further comprising diagnosing the patient as having a condition characterized by neuronal injury or neuronal damage if the protein level of Wnt5a protein in the sample is higher than that of the control sample.

24. A method for determining the presence of neuronal injury or neuronal damage in a patient, comprising:
obtaining a sample from the patient;
measuring the protein level of Wnt5a protein in the sample and comparing the protein level of Wnt5a protein in the sample to a control sample;
wherein the patient is treated if the protein level of Wnt5a protein is changed compared to the control.

25. The method of claim 24, wherein a higher protein level of Wnt5a protein in the sample relative to the control sample is indicative of a neurodegenerative disease.

26. A method for determining the prognosis of a patient suffering from a condition characterized by neuronal injury or neuronal damage, comprising:
obtaining a sample from a patient;
measuring the expression level of Wnt5a protein in the sample and comparing the expression level of Wnt5a protein in the sample to a control sample; and
determining the prognosis of the patient.

27. The method of claim 1, further comprising repeating the measuring step at one or more intervals.

28. The method of any one of claims 22, 24, or 26, wherein measuring comprises Western blot, ELISA (enzyme linked immunosorbent assay), radioimmunoassay analysis (RIA), radial immunodiffusion, Ouchterlony immunodiffusion, rocket immunoelectrophoresis, tissue immunohistochemistry, immunoprecipitation assays, complement fixation assays, flow cytometry, and protein chip (protein microarray), capillary western blot, protein MS, Protein sequencing, HPLC, or gas chromatography.

29. The method of any one of claims 22, 24, or 26, wherein the sample comprises blood, cerebrospinal fluid, tissue biopsies or a combination thereof.

30. The method of claim 29, wherein the blood sample is separated into plasma before measuring.

31. The method of any one of claims 22, 24, or 26, wherein the control sample comprises a sample from a normal subject.

32. The method of any one of claims 22, 24, or 26, wherein the control sample comprises a sample isolated from the patient prior to the onset of the neuronal injury or neuronal damage.

33. The method of any one of claims 22, 24, or 26, wherein the condition is stroke.

34. The method of claim 33, wherein the protein level of Wnt5a protein is measured within seven days, five days, or three days of the stroke event.

35. The method of any one of claims 22, 24, or 26, wherein the neuronal injury or neuronal damage comprises uncompensated oxidative stress.

36. A diagnostic kit for determining brain injury status in a patient comprising:
a substrate for collecting a sample from a patient; and
means for measuring the protein level of Wnt5a protein.

37. The diagnostic kit of claim 36, wherein the means for measuring comprises Western blot, ELISA (enzyme linked immunosorbent assay), radioimmunoassay analysis (RIA), radial immunodiffusion, Ouchterlony immunodiffusion, rocket immunoelectrophoresis, tissue immunohistochemistry, immunoprecipitation assays, complement fixation assays, flow cytometry, and protein chip (protein microarray), capillary western blot, protein MS, Protein sequence, HPLC, or gas chromatography.
